(19) United States
(12) Patent Application Publication
(10) Pub. No.: US 2013/0281413 A1
(43) Pub. Date: Oct. 24, 2013

(54) Salicylate Conjugates Useful For Treating Metabolic Disorders

(71) Applicant: Genmedica Therapeutics SL, Barcelona (ES)

(72) Inventors: Alec Mian, Barcelona (ES); Luc Marti Clauzel, Barcelona (ES); Eric Mayoux, Eschau (FR); Silvia Garcia Vicente, Sant Feliu de Llobregat (ES); Marta Serrano Munoz, Barcelona (ES); Antonio Zorzano Olarte, Barcelona (ES)

(21) Appl. No.: 13/829,266

(22) Filed: Mar. 14, 2013

Related U.S. Application Data
(63) Continuation of application No. 12/465,201, filed on May 13, 2009.
(60) Provisional application No. 61/052,839, filed on May 13, 2008.

(51) Int. Cl. A61K 47/48

(52) U.S. Cl. CPC A61K 47/481
USPC 514/166; 514/159

(57) ABSTRACT

The present invention is directed to methods for treating metabolic disorders with compounds that are conjugates. The conjugates of the present invention are comprised of salicylic acid, triflusal, diflusinal, salsalate, IMD-0354, ibuprofen, diclofenac, licofelone, or HTB, and one or more antioxidants.

### CROSS-REFERENCE TO RELATED APPLICATIONS

This application is a continuation of U.S. patent application Ser. No. 12/465,201, which claims priority to U.S. Provisional Patent Application Ser. No. 61/052,839, filed May 13, 2008, the disclosure of each of which is hereby incorporated herein by reference in its entirety.

### BACKGROUND

Oxidative stress and inflammation are implicated in the pathogenesis of metabolic diseases, diabetes, obesity, dyslipidemia and their associated cardiovascular complications. For example, oxidative stress is a common pathogenic factor leading to insulin resistance, β-cell dysfunction, impaired glucose tolerance, and type 2 diabetes mellitus. With regard to inflammation, clinical studies suggest that acute hyperglycemia results in elevated levels of circulating inflammatory cytokines such as TNFα, IL6, and IL18.

During hyperglycemia and/or hyperlipidemia, mitochondria generate cellular energy through TCA cycle activity and the associated electron transport chain of the inner mitochondrial membrane. However, while mitochondria generate elevated ATP production, mitochondria can also generate significant reactive oxygen species (ROS) and reactive nitrogen species (RNS). Cells are equipped with several antioxidant enzymes to neutralize ROS and RNS. For example, superoxide anions are enzymatically converted to hydrogen peroxide by a manganese superoxide dismutase (MnSOD) within mitochondria. Hydrogen peroxide can then be rapidly removed by the mitochondrial enzyme glutathione (GSH) peroxidase. A further antioxidant enzyme, catalase, is the hydrogen peroxide detoxifying enzyme founded exclusively in peroxisomes. Glutathione (GSH) is probably the most important defense with which the cell is equipped, for scavenging ROS generated by mitochondria metabolism and excess free radicals produced secondary to hyperglycemia and hyperlipidemia.

However, while cells have a number of available anti-oxidant mechanisms, damage most likely occurs when the ROS is excessive and/or anti-oxidant pathways are overwhelmed as is frequently the case in diabetes. In diabetic patients, the levels of antioxidant enzymes responsible for scavenging free radicals are diminished. Glutathione pools become depleted in diabetic patients following frequent and severe hyperglycemic episodes. It is now widely accepted that overproduction of reactive oxygen species (ROS) contributes to cell and tissue dysfunction and damage caused by glucolipotoxicity in diabetes, insulin resistance, and obesity.

In particular, compared to several other cells of the body, pancreatic β-cells have relatively low levels of free radical detoxification and redox regulating enzymes such as superoxide dismutase, glutathione peroxidase, catalase and thioredoxin. The consequence of limited scavenging systems is that ROS concentration in β-cells may increase rapidly, damaging the β-cells. Thus, under hyperglycemic conditions, the production of ROS, and subsequent oxidative stress, contributes to β-cell deterioration observed in type 2 diabetes.

ROS is also considered a strong stimulus for the release of cytokines and increased superoxide can promote inflammation through NF-kB activation. Thus the role of oxidative stress and associated activation of NF-kB leading to chronic inflammation and insulin resistance is essential in the processes implicated in the pathogenesis of diabetes and its progression. Administration of glutathione, a powerful antioxidant, completely suppresses cytokine elevation, providing further support that an oxidative stress mechanism mediates the inflammatory effects of hyperglycemia in humans.

Salicylates, or aspirin-like drugs, are some of the most commonly used anti-inflammatory agents. For more than two decades, the anti-inflammatory properties of aspirin have been almost exclusively attributed to blocking prostaglandin synthesis via inhibition of cyclo-oxygenase activity. Recently, aspirin and sodium salicylate have been found to inhibit the activation of the transcription factor NF-kB. High doses of salicylate are thought to inhibit NF-kB and its upstream activator, the IKB kinase β (IKKβ).

Also, high doses of salicylic acid lower blood glucose levels. Recent studies report that diabetic animals given salicylates or Salsalate showed a decrease in IKKβ activity, accompanied by improvement in insulin sensitivity. High doses of Salicylate (120 mg/kg/day) administered by subcutaneous infusion in Zucker fa/fa rats or ob/ob mice for 3-4 weeks exhibited anti-diabetic effects, reduction in fasting blood glucose, and glucose tolerance improvement. Beneficial effects of high doses of salicylic acid have been recently reported in human diabetic patients treated with 4.5 g/day of salsalate. However, at this high dose, side effects, such as tinnitus, are enhanced by 66% and the long term risk of gastric bleeding and ulceration is also increased.

Thus, there remains a need in the art for compounds for treating metabolic disorders by way of ameliorating the inflammatory and oxidative processes associated with such disorders, particularly diabetes.

### SUMMARY OF THE INVENTION

The present invention relates to conjugates comprised of salicylic acid and an anti-oxidant agent. The conjugates of the present invention are useful for treating atherosclerosis, neuropathy, nephropathy, retinopathy, inflammatory disorders, cardiovascular diseases, and metabolic disorders, such as any form of diabetes mellitus including type I and type II diabetes, metabolic syndrome, hyperglycemia, and insulin sensitivity. The conjugates are also useful for reducing advanced glycated end products (AGEs), ROS, lipid peroxidation, tissue and plasma TNFα and IL6 levels, and for delaying or preventing cardiovascular complications associated with atherosclerosis. Also, the conjugates of the present invention are useful for protecting pancreatic β-cells, preventing their impairment or failure and subsequent lower insulin secretion. In particular, the present invention is exemplified by the use of salnacedin, a conjugate of salicylic acid and N-acetylcysteine, for treating the disorders disclosed herein.

The compounds of the present invention, in particular Example 1 (salnacedin), show additive or synergistic effects relative to treatment with an antioxidant agent alone or an anti-inflammatory agent alone. The additive or synergistic effect improves the anti-diabetic effect while reducing side effects associated with monotherapy. In particular, treatment with Example 1 or salnacedin improves anti-diabetic effects while lowering the risk of gastric bleeding, associated with salicylic acid, and/or tinnitus, associated with N-acetylcysteine.

The present invention also provides methods for treating atherosclerosis, neuropathy, nephropathy, retinopathy, inflammatory disorders, cardiovascular diseases, and metabolic disorders in a mammal or patient which includes the step of administering to the mammal or patient in need of such treatment a therapeutically effective amount of a compound of Formula (I)

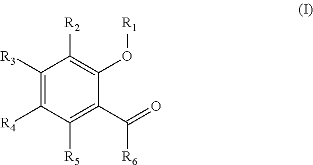

or a pharmaceutically acceptable salt thereof, wherein

R₁ is hydrogen, (C₁-C₆)alkylcarbonyl, or A;

R₂, R₃, R₄, and R₅ are independently hydrogen, (C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ₁Z₂, or (NZ₁Z₂)carbonyl, wherein the phenyl is optionally substituted with 1, 2, 3, 4, or 5 groups that are independently (C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ₃Z₄, (NZ₃Z₄)carbonyl;

Z₁, Z₂, Z₃, and Z₄ are independently hydrogen, (C₁-C₆)alkyl, or (C₁-C₆)alkylcarbonyl;

R₆ is —NZ₅Z₆,

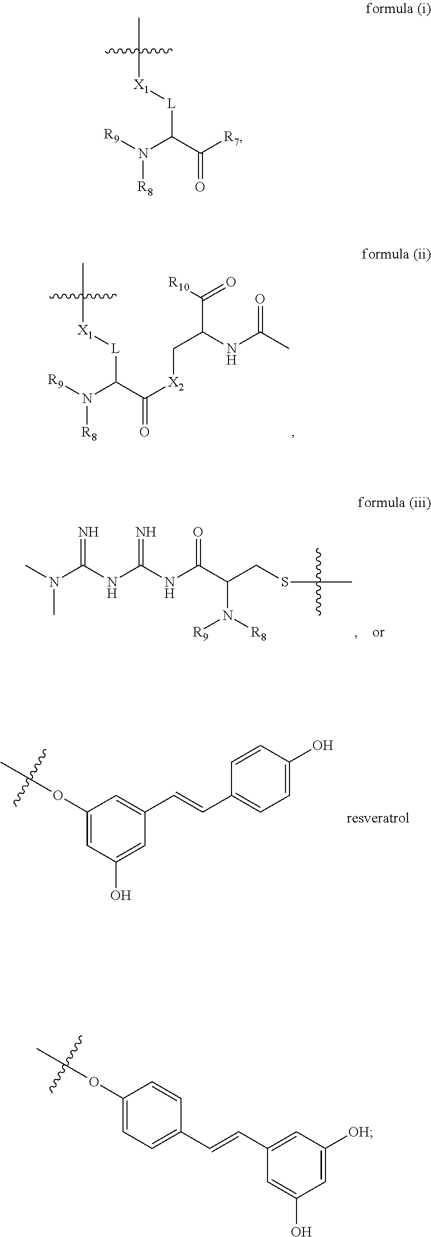

Z₅ and Z₆ are independently hydrogen, (C₁-C₆)alkyl, (C₁-C₆)alkylcarbonyl, phenyl, phenyl(CH₂)—, or phenyl(CH₂)₂—, wherein the phenyl is optionally substituted with 1, 2, 3, 4, or 5 groups that are independently (C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ₇Z₈, or (NZ₇Z₈)carbonyl;

Z₇ and Z₈ are independently hydrogen, (C₁-C₆)alkyl, or (C₁-C₆)alkylcarbonyl;

R₇ is (C₁-C₆)alkoxy, (C₁-C₆)alkyl, (C₁-C₆)alkylthio, hydroxy, or —NZ₉Z₁₀;

R₈ is hydrogen or (C₁-C₆)alkyl;

R₉ is hydrogen, (C₁-C₆)alkyl, or (C₁-C₆)alkylcarbonyl;

R₁₀ is (C₁-C₆)alkoxy, (C₁-C₆)alkyl, (C₁-C₆)alkylthio, hydroxy, or —NZ₉Z₁₀;

Z₉ and Z₁₀ are independently hydrogen, (C₁-C₆)alkyl, or (C₁-C₆)alkylcarbonyl;

X₁ and X₂ are independently O or S;

L is (C₁-C₆)alkylene;

A is

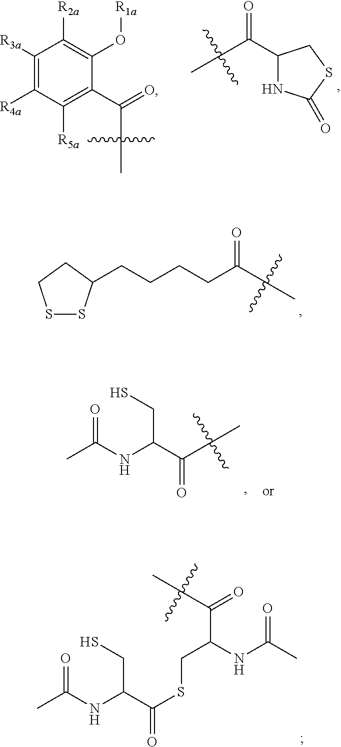

R₁ₐ is hydrogen, (C₁-C₆)alkylcarbonyl, or B;

R₂ₐ, R₃ₐ, R₄ₐ, and R₅ₐ are independently hydrogen, (C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ₁ₐZ₂ₐ, or (NZ₁ₐZ₂ₐ)carbonyl, wherein the phenyl is optionally substituted with 1, 2, 3, 4, or 5 groups that are independently C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ₃ₐZ₄ₐ, or (NZ₃ₐZ₄ₐ)carbonyl;

Z₁ₐ, Z₂ₐ, Z₃ₐ, and Z₄ₐ are independently hydrogen, (C₁-C₆)alkyl, or (C₁-C₆)alkylcarbonyl;

B is

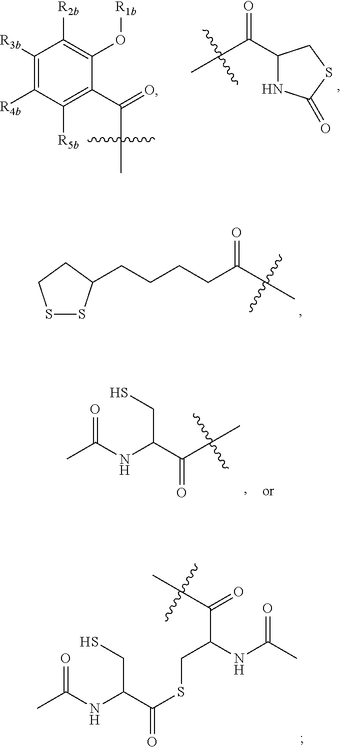

R_{1b} is hydrogen, (C₁-C₆)alkylcarbonyl, or C;

R_{2b}, R_{3b}, R_{4b}, and R_{5b} are independently hydrogen, (C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ_{1b}Z_{2b}, or (NZ_{1b}Z_{2b})carbonyl, wherein the phenyl is optionally substituted with 1, 2, 3, 4, or 5 groups that are independently C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ_{3b}Z_{4b}, or (NZ_{3b}Z_{4b})carbonyl;

Z_{1b}, Z_{2b}, Z_{3b}, and Z_{4b} are independently hydrogen, (C₁-C₆)alkyl, or (C₁-C₆)alkylcarbonyl; and

C is

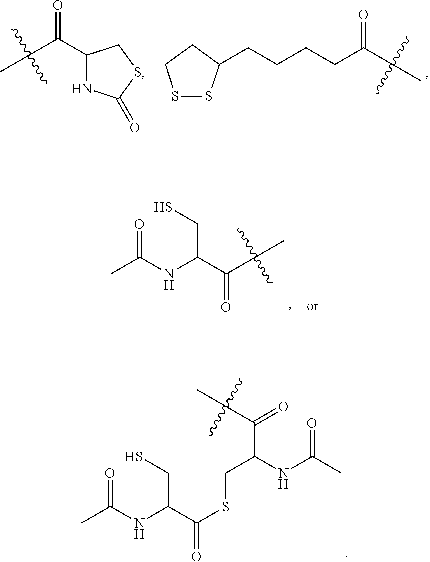

In another aspect, the present invention provides methods for treating dyslipidemia, insulin resistance, β-cell dysfunction, hyperglycemia, metabolic syndrome, and any form of diabetes mellitus including type I and type II diabetes, in a patient comprising administering to the patient in need of such treatment a therapeutically effective amount of a compound of Formula (I), or pharmaceutically acceptable salt thereof.

In another aspect, the present invention provides methods for treating atherosclerosis, neuropathy, nephropathy, retinopathy, inflammatory disorders, cardiovascular diseases, and metabolic disorders in a mammal or patient comprising administering to the mammal or patient a therapeutically effective amount of a pharmaceutically acceptable composition wherein the composition comprises a compound of Formula (I), or pharmaceutically acceptable salt thereof, and at least one pharmaceutically acceptable carrier.

In another aspect, the present invention provides uses for compounds of Formula (I) for preparing, or for the manufacture of, a medicament for treating metabolic disorders in a mammal or patient.

Specific embodiments of the present invention will become evident from the following more detailed description of certain preferred embodiments and the claims.

### BRIEF DESCRIPTION OF THE DRAWINGS

FIG. 1 is directed to the chemical stability of conjugates of the present invention in neutral, acidic, and basic solutions. The conjugates were tested in their free acid form and as lysine salts and include: salicylic acid-(L) N-acetyl cysteine (GMC-3a), diflunisal-(L) N-acetyl cysteine (GMC-3b), and dexibuprofen-(L) N-acetyl cysteine (GMC-3d).

FIGS. 2-4 are graphical illustrations of the cleavage efficiency for salicylic acid-(L) N-acetyl cysteine (GMC-3a) and diflunisal-(L) N-acetyl cysteine (GMC-3b) in rat and human.

FIG. 5 is a graphical illustration of the cleavage efficiency for salicylic acid-(L) N-acetyl cysteine (GMC-3a), diflunisal-(L) N-acetyl cysteine (GMC-3b) in vivo in rats.

FIG. 6 is a graphical illustration of the effects of salicylic acid-(L) N-acetyl cysteine (GMC-1.3a) and diflunisal-(L) N-acetyl cysteine (GMC-1.3b), as lysine salts, at protecting beta-cells in vivo in the alloxan model. The alloxan model is a well known model of β-cell dysfunction that mimics the biochemical events involved in type 2 diabetes, including inflammation and oxidative stress. The results in FIG. 6 indicate that both conjugates reduce the effect of alloxan on β-cells. Further, the preservation of insulin levels in alloxan rats treated with GMC-3a, as shown in FIG. 6, indicates a pancreatic beta cell protection mechanism of action.

FIG. 7 is a graphical illustration of the comparative effects of the conjugate salicylic acid-(L) N-acetyl cysteine (GMC-1.3a) as the lysine salt, salicylate, and NAC, on free fatty acid and triglyceride levels in db/db mice (ip administration).

FIGS. 8-10 is a graphical illustration of the acute and chronic effects of the conjugate diflunisal-(L) N-acetyl cysteine (GMC-1.3b), as the lysine salt, on hyperglycemia in db/db mice subsequent (oral administration).

FIG. 11 is a graphical illustration of the effect of the conjugate diflunisal-(L) N-acetyl cysteine (GMC-1.3b), as the lysine salt, on plasma insulin levels in db/db (oral administration).

FIG. 12 is a graphical illustration of the effects of the conjugate diflunisal-(L) N-acetyl cysteine (GMC-1.3b), as the lysine salt, on free fatty acid and triglyceride levels in db/db mice (chronic oral administration).

FIG. 13 is a graphical illustration of the effects of the conjugates salicylic acid-(L) N-acetyl cysteine (GMC-3a) and diflunisal-(L) N-acetyl cysteine (GMC-3b) on body weight gain in db/db mice (chronic oral administration).

FIG. 14 is a graphical illustration of the effects of the conjugates salicylic acid-(L) N-acetyl cysteine (GMC-3a) and diflunisal-(L) N-acetyl cysteine (GMC-3b) on fluid and food intake in db/db mice (chronic oral administration).

FIG. 15 illustrates the protocol used in FIGS. 8, 9, 10, 11, 12, 13, and 14.

### DETAILED DESCRIPTION

The present invention provides compounds, reagents, pharmaceutical compositions and methods for treating atherosclerosis, neuropathy, nephropathy, retinopathy, inflammatory disorders, cardiovascular diseases, and metabolic disorders in a mammal or patient comprising administering to the mammal or patient in need of such treatment a therapeutically effective amount of a compound of Formula (I), or a pharmaceutically acceptable salt thereof, wherein R₁ is hydrogen or acetyl; R₂, R₃, R₄, and R₅ are independently hydrogen, trifluoromethyl, or 2,4-difluorophenyl; R₆ is formula (i); and R₇, R₈, R₉, X₁, and L are as defined in Formula (I) of the Summary section.

In certain embodiments, the inventive methods include treating dyslipidemia, insulin resistance, β-cell dysfunction, hyperglycemia, metabolic syndrome, and any form of diabetes mellitus including type I and type II diabetes, in a patient comprising administering to the patient in need of such treatment a therapeutically effective amount of a compound of Formula (I), or a pharmaceutically acceptable salt thereof, wherein R₁ is hydrogen or acetyl; R₂, R₃, R₄, and R₅ are independently hydrogen, trifluoromethyl, or 2,4-difluorophenyl; R₆ is formula (i); and R₇, R₈, R₉, X₁, and L are as defined in Formula (I) of the Summary section.

In certain embodiments, the present invention provides methods for reducing advanced glycated end products and/or lipid peroxidation including, but not limited to, oxidation of low-density lipoproteins in a mammal or patient comprising administering to the mammal or patient in need of such treatment a therapeutically effective amount of a compound of Formula (I), or a pharmaceutically acceptable salt thereof, wherein R₁ is hydrogen or acetyl; R₂, R₃, R₄, and R₅ are independently hydrogen, trifluoromethyl, or 2,4-difluorophenyl; R₆ is formula (i); and R₇, R₈, R₉, X₁, and L are as defined in Formula (I) of the Summary section.

In certain embodiments, the inventive methods include treating dyslipidemia, insulin resistance, β-cell dysfunction, hyperglycemia, metabolic syndrome, and any form of diabetes mellitus including type I and type II diabetes, in a patient comprising administering to the patient in need of such treatment a therapeutically effective amount of a pharmaceutical composition comprising at least one pharmaceutically acceptable carrier and a compound of Formula (I), or a pharmaceutically acceptable salt thereof, wherein R₁ is hydrogen or acetyl; R₂, R₃, R₄, and R₅ are independently hydrogen, trifluoromethyl, or 2,4-difluorophenyl; R₆ is formula (i); and R₇, R₈, R₉, X₁, and L are as defined in Formula (I) of the Summary section.

In certain embodiments, the present invention provides methods for reducing advanced glycated end products and lipid peroxidation including, but not limited to, oxidation of low-density lipoproteins, in a patient comprising administering to the patient in need of such treatment a therapeutically effective amount of a pharmaceutical composition comprising at least one pharmaceutically acceptable carrier and a compound of Formula (I), or a pharmaceutically acceptable salt thereof, wherein R₁ is hydrogen or acetyl; R₂, R₃, R₄, and R₅ are independently hydrogen, trifluoromethyl, or 2,4-difluorophenyl; R₆ is formula (i); and R₇, R₈, R₉, X₁, and L are as defined in Formula (I) of the Summary section.

In another aspect of the present invention, a method is provided for treating atherosclerosis, neuropathy, nephropathy, retinopathy, inflammatory disorders, cardiovascular diseases, and metabolic disorders in a mammal or patient which includes the step of administering to the mammal or patient in need of such treatment a therapeutically effective amount of a compound of Formula (I), or a pharmaceutically acceptable salt thereof, wherein R₁ is hydrogen or acetyl; R₂, R₃, R₄, and R₅ are independently hydrogen, trifluoromethyl, or 2,4-difluorophenyl; R₆ is formula (i); R₇ is (C₁-C₆)alkoxy or hydroxy; R₉ is hydrogen; R₉ is (C₁-C₆)alkylcarbonyl; X₁ is S; and L is CH₂.

In certain embodiments, the inventive methods include treating dyslipidemia, insulin resistance, β-cell dysfunction, hyperglycemia, metabolic syndrome, and any form of diabetes mellitus including type I and type II diabetes, in a patient comprising administering to the patient in need of such treatment a therapeutically effective amount of a compound of Formula (I), or a pharmaceutically acceptable salt thereof, wherein R₁ is hydrogen or acetyl; R₂, R₃, R₄, and R₅ are independently hydrogen, trifluoromethyl, or 2,4-difluorophenyl; R₆ is formula (i); R₇ is (C₁-C₆)alkoxy or hydroxy; R₉ is hydrogen; R₉ is (C₁-C₆)alkylcarbonyl; X₁ is S; and L is CH₂.

In certain embodiments, the present invention provides methods for reducing advanced glycated end products and/or lipid peroxidation including, but not limited to, oxidation of low-density lipoproteins in a mammal or patient comprising administering to the mammal or patient in need of such treatment a therapeutically effective amount of a compound of Formula (I), or a pharmaceutically acceptable salt thereof, wherein R₁ is hydrogen or acetyl; R₂, R₃, R₄, and R₅ are independently hydrogen, trifluoromethyl, or 2,4-difluorophenyl; R₆ is formula (i); R₇ is (C₁-C₆)alkoxy or hydroxy; R₉ is hydrogen; R₉ is (C₁-C₆)alkylcarbonyl; X₁ is S; and L is CH₂.

In certain embodiments, the inventive methods include treating dyslipidemia, insulin resistance, β-cell dysfunction, hyperglycemia, metabolic syndrome, and any form of diabetes mellitus including type I and type II diabetes, in a patient comprising administering to the patient in need of such treatment a therapeutically effective amount of a pharmaceutical composition comprising at least one pharmaceutically acceptable carrier and a compound of Formula (I), or a pharmaceutically acceptable salt thereof, wherein R₁ is hydrogen or acetyl; R₂, R₃, R₄, and R₅ are independently hydrogen, trifluoromethyl, or 2,4-difluorophenyl; R₆ is formula (i); R₇ is (C₁-C₆)alkoxy or hydroxy; R₉ is hydrogen; R₉ is (C₁-C₆)alkylcarbonyl; X₁ is S; and L is CH₂.

In certain embodiments, the present invention provides methods for reducing advanced glycated end products and lipid peroxidation including, but not limited to, oxidation of low-density lipoproteins, in a patient comprising administering to the patient in need of such treatment a therapeutically effective amount of a pharmaceutical composition comprising at least one pharmaceutically acceptable carrier and a compound of Formula (I), or a pharmaceutically acceptable salt thereof, wherein R₁ is hydrogen or acetyl; R₂, R₃, R₄, and R₅ are independently hydrogen, trifluoromethyl, or 2,4-difluorophenyl; R₆ is formula (i); R₇ is (C₁-C₆)alkoxy or hydroxy; R₉ is hydrogen; R₉ is (C₁-C₆)alkylcarbonyl; X₁ is S; and L is CH₂.

In another aspect of the present invention, a method is provided for treating atherosclerosis, neuropathy, nephropathy, retinopathy, inflammatory disorders, cardiovascular diseases, and metabolic disorders in a mammal or patient which includes the step of administering to the mammal or patient in need of such treatment a therapeutically effective amount of a compound of Formula (I), or a pharmaceutically acceptable salt thereof, wherein R₁ is hydrogen or acetyl; R₂, R₃, R₄, and R₅ are independently hydrogen, trifluoromethyl, or 2,4-difluorophenyl; R₆ is formula (i); R₇ is ethoxy, methoxy, or hydroxy; R₉ is hydrogen; R₉ is acetyl; X₁ is S; and L is CH₂.

In certain embodiments, the inventive methods include treating dyslipidemia, insulin resistance, β-cell dysfunction, hyperglycemia, metabolic syndrome, and any form of diabetes mellitus including type I and type II diabetes, in a patient comprising administering to the patient in need of such treatment a therapeutically effective amount of a compound of Formula (I), or a pharmaceutically acceptable salt thereof, wherein R₁ is hydrogen or acetyl; R₂, R₃, R₄, and R₅ are independently hydrogen, trifluoromethyl, or 2,4-difluorophenyl; R₆ is formula (i); R₇ is ethoxy, methoxy, or hydroxy; R₉ is hydrogen; R₉ is acetyl; X₁ is S; and L is CH₂.

In certain embodiments, the present invention provides methods for reducing advanced glycated end products and/or lipid peroxidation including, but not limited to, oxidation of low-density lipoproteins in a mammal or patient comprising administering to the mammal or patient in need of such treatment a therapeutically effective amount of a compound of Formula (I), or a pharmaceutically acceptable salt thereof, wherein R₁ is hydrogen or acetyl; R₂, R₃, R₄, and R₅ are independently hydrogen, trifluoromethyl, or 2,4-difluorophenyl; R₆ is formula (i); R₇ is ethoxy, methoxy, or hydroxy; R₉ is hydrogen; R₉ is acetyl; X₁ is S; and L is CH₂.

In certain embodiments, the inventive methods include treating dyslipidemia, insulin resistance, β-cell dysfunction, hyperglycemia, metabolic syndrome, and any form of diabetes mellitus including type I and type II diabetes, in a patient comprising administering to the patient in need of such treatment a therapeutically effective amount of a pharmaceutical composition comprising at least one pharmaceutically acceptable carrier and a compound of Formula (I), or a pharmaceutically acceptable salt thereof, wherein R₁ is hydrogen or acetyl; R₂, R₃, R₄, and R₅ are independently hydrogen, trifluoromethyl, or 2,4-difluorophenyl; R₆ is formula (i); R₇ is ethoxy, methoxy, or hydroxy; R₉ is hydrogen; R₉ is acetyl; X₁ is S; and L is CH₂.

In certain embodiments, the present invention provides methods for reducing advanced glycated end products and lipid peroxidation including, but not limited to, oxidation of low-density lipoproteins, in a patient comprising administering to the patient in need of such treatment a therapeutically effective amount of a pharmaceutical composition comprising at least one pharmaceutically acceptable carrier and a compound of Formula (I), or a pharmaceutically acceptable salt thereof, wherein R₁ is hydrogen or acetyl; R₂, R₃, R₄, and R₅ are independently hydrogen, trifluoromethyl, or 2,4-difluorophenyl; R₆ is formula (i); R₇ is ethoxy, methoxy, or hydroxy; R₉ is hydrogen; R₉ is acetyl; X₁ is S; and L is CH₂.

In accordance with the present invention, a method is provided for treating atherosclerosis, neuropathy, nephropathy, retinopathy, inflammatory disorders, cardiovascular diseases, and metabolic disorders in a mammal or patient which includes the step of administering to the mammal or patient in need of such treatment a therapeutically effective amount of a compound of Formula (I), or a pharmaceutically acceptable salt thereof, wherein R₁ is hydrogen or acetyl; R₂, R₃, R₄, and R₅ are independently hydrogen, trifluoromethyl, or 2,4-difluorophenyl; and R₆ is (L) N-acetylcysteine.

In certain embodiments, the inventive methods include treating dyslipidemia, insulin resistance, β-cell dysfunction, hyperglycemia, metabolic syndrome, and any form of diabetes mellitus including type I and type II diabetes, in a patient comprising administering to the patient in need of such treatment a therapeutically effective amount of a compound of Formula (I), or a pharmaceutically acceptable salt thereof, wherein R₁ is hydrogen or acetyl; R₂, R₃, R₄, and R₅ are independently hydrogen, trifluoromethyl, or 2,4-difluorophenyl; and R₆ is (L) N-acetylcysteine.

In certain embodiments, the present invention provides methods for reducing advanced glycated end products and/or lipid peroxidation including, but not limited to, oxidation of low-density lipoproteins in a mammal or patient comprising administering to the mammal or patient in need of such treatment a therapeutically effective amount of a compound of Formula (I), or a pharmaceutically acceptable salt thereof, wherein R₁ is hydrogen or acetyl; R₂, R₃, R₄, and R₅ are independently hydrogen, trifluoromethyl, or 2,4-difluorophenyl; and R₆ is (L) N-acetylcysteine.

In certain embodiments, the inventive methods include treating dyslipidemia, insulin resistance, β-cell dysfunction, hyperglycemia, metabolic syndrome, and any form of diabetes mellitus including type I and type II diabetes, in a patient comprising administering to the patient in need of such treatment a therapeutically effective amount of a pharmaceutical composition comprising at least one pharmaceutically acceptable carrier and a compound of Formula (I), or a pharmaceutically acceptable salt thereof, wherein R₁ is hydrogen or acetyl; R₂, R₃, R₄, and R₅ are independently hydrogen, trifluoromethyl, or 2,4-difluorophenyl; and R₆ is (L) N-acetylcysteine.

In certain embodiments, the present invention provides methods for reducing advanced glycated end products and lipid peroxidation including, but not limited to, oxidation of low-density lipoproteins, in a patient comprising administering to the patient in need of such treatment a therapeutically effective amount of a pharmaceutical composition comprising at least one pharmaceutically acceptable carrier and a compound of Formula (I), or a pharmaceutically acceptable salt thereof, wherein R₁ is hydrogen or acetyl; R₂, R₃, R₄, and R₅ are independently hydrogen, trifluoromethyl, or 2,4-difluorophenyl; and R₆ is (L) N-acetylcysteine.

In accordance with the present invention, a method is provided for treating atherosclerosis, neuropathy, nephropathy, retinopathy, inflammatory disorders, cardiovascular diseases, and metabolic disorders in a mammal or patient which includes the step of administering to the mammal or patient in need of such treatment a therapeutically effective amount of a compound of Formula (I), or a pharmaceutically acceptable salt thereof, wherein R₁ is hydrogen or acetyl; R₂, R₃, R₄, and R₅ are independently hydrogen, halo(C₁-C₆)alkyl, or halogen; R₆ is —NZ₅Z₆; Z₅ is hydrogen; Z₆ is hydrogen, (C₁-C₆)alkyl, (C₁-C₆)alkylcarbonyl, phenyl, phenyl(CH₂)—, or phenyl(CH₂)₂—, wherein the phenyl is optionally substituted with 1, 2, 3, 4, or 5 groups that are independently (C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ₇Z₈, or (NZ₇Z₈)carbonyl; and Z₇ and Z₈ are independently hydrogen, (C₁-C₆)alkyl, or (C₁-C₆)alkylcarbonyl.

In certain embodiments, the inventive methods include treating dyslipidemia, insulin resistance, β-cell dysfunction, hyperglycemia, metabolic syndrome, and any form of diabetes mellitus including type I and type II diabetes, in a patient comprising administering to the patient in need of such treatment a therapeutically effective amount of a compound of Formula (I), or a pharmaceutically acceptable salt thereof, wherein R₁ is hydrogen or acetyl; R₂, R₃, R₄, and R₅ are independently hydrogen, halo(C₁-C₆)alkyl, or halogen; R₆ is —NZ₅Z₆; Z₅ is hydrogen; Z₆ is hydrogen, (C₁-C₆)alkyl, (C₁-C₆)alkylcarbonyl, phenyl, phenyl(CH₂)—, or phenyl(CH₂)₂—, wherein the phenyl is optionally substituted with 1, 2, 3, 4, or 5 groups that are independently (C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ₇Z₈, or (NZ₇Z₈)carbonyl; and Z₇ and Z₈ are independently hydrogen, (C₁-C₆)alkyl, or (C₁-C₆)alkylcarbonyl.

In certain embodiments, the present invention provides methods for reducing advanced glycated end products and/or lipid peroxidation including, but not limited to, oxidation of low-density lipoproteins in a mammal or patient comprising administering to the mammal or patient in need of such treatment a therapeutically effective amount of a compound of Formula (I), or a pharmaceutically acceptable salt thereof, wherein R₁ is hydrogen or acetyl; R₂, R₃, R₄, and R₅ are independently hydrogen, halo(C₁-C₆)alkyl, or halogen; R₆ is —NZ₅Z₆; Z₅ is hydrogen; Z₆ is hydrogen, (C₁-C₆)alkyl, (C₁-C₆)alkylcarbonyl, phenyl, phenyl(CH₂)—, or phenyl(CH₂)₂—, wherein the phenyl is optionally substituted with 1, 2, 3, 4, or 5 groups that are independently (C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ₇Z₈, or (NZ₇Z₈)carbonyl; and Z₇ and Z₈ are independently hydrogen, (C₁-C₆)alkyl, or (C₁-C₆)alkylcarbonyl.

In certain embodiments, the inventive methods include treating dyslipidemia, insulin resistance, β-cell dysfunction, hyperglycemia, metabolic syndrome, and any form of diabetes mellitus including type I and type II diabetes, in a patient comprising administering to the patient in need of such treatment a therapeutically effective amount of a pharmaceutical composition comprising at least one pharmaceutically acceptable carrier and a compound of Formula (I), or a pharmaceutically acceptable salt thereof, wherein R₁ is hydrogen or acetyl; R₂, R₃, R₄, and R₅ are independently hydrogen, halo(C₁-C₆)alkyl, or halogen; R₆ is —NZ₅Z₆; Z₅ is hydrogen; Z₆ is hydrogen, (C₁-C₆)alkyl, (C₁-C₆)alkylcarbonyl, phenyl, phenyl(CH₂)—, or phenyl(CH₂)₂—, wherein the phenyl is optionally substituted with 1, 2, 3, 4, or 5 groups that are independently (C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ₇Z₈, or (NZ₇Z₈)carbonyl; and Z₇ and Z₈ are independently hydrogen, (C₁-C₆)alkyl, or (C₁-C₆)alkylcarbonyl.

In certain embodiments, the present invention provides methods for reducing advanced glycated end products and lipid peroxidation including, but not limited to, oxidation of low-density lipoproteins, in a patient comprising administering to the patient in need of such treatment a therapeutically effective amount of a pharmaceutical composition comprising at least one pharmaceutically acceptable carrier and a compound of Formula (I), or a pharmaceutically acceptable salt thereof, wherein R₁ is hydrogen or acetyl; R₂, R₃, R₄, and R₅ are independently hydrogen, halo(C₁-C₆)alkyl, or halogen; R₆ is —NZ₅Z₆; Z₅ is hydrogen; Z₆ is hydrogen, (C₁-C₆)alkyl, (C₁-C₆)alkylcarbonyl, phenyl, phenyl(CH₂)—, or phenyl(CH₂)₂, wherein the phenyl is optionally substituted with 1, 2, 3, 4, or 5 groups that are independently (C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ₇Z₈, or (NZ₇Z₈)carbonyl; and Z₇ and Z₈ are independently hydrogen, (C₁-C₆)alkyl, or (C₁-C₆)alkylcarbonyl.

The present invention further provides methods for treating atherosclerosis, neuropathy, nephropathy, retinopathy, inflammatory disorders, cardiovascular diseases, and metabolic disorders in a mammal or patient which includes the step of administering to the mammal or patient in need of such treatment a therapeutically effective amount of a compound of Formula (I), or a pharmaceutically acceptable salt thereof, wherein R₁ is hydrogen or acetyl; R₂, R₃, R₄, and R₅ are independently hydrogen, halo(C₁-C₆)alkyl, or halogen; R₆ is —NZ₅Z₆; Z₅ is hydrogen; Z₆ is hydrogen, (C₁-C₆)alkyl, (C₁-C₆)alkylcarbonyl.

In certain embodiments, the inventive methods include treating dyslipidemia, insulin resistance, β-cell dysfunction, hyperglycemia, metabolic syndrome, and any form of diabetes mellitus including type I and type II diabetes, in a patient comprising administering to the patient in need of such treatment a therapeutically effective amount of a compound of Formula (I), or a pharmaceutically acceptable salt thereof, wherein R₁ is hydrogen or acetyl; R₂, R₃, R₄, and R₅ are independently hydrogen, halo(C₁-C₆)alkyl, or halogen; R₆ is —NZ₅Z₆; Z₅ is hydrogen; Z₆ is hydrogen, (C₁-C₆)alkyl, (C₁-C₆)alkylcarbonyl.

In certain embodiments, the present invention provides methods for reducing advanced glycated end products and/or lipid peroxidation including, but not limited to, oxidation of low-density lipoproteins in a mammal or patient comprising administering to the mammal or patient in need of such treatment a therapeutically effective amount of a compound of Formula (I), or a pharmaceutically acceptable salt thereof, wherein R₁ is hydrogen or acetyl; R₂, R₃, R₄, and R₅ are independently hydrogen, halo(C₁-C₆)alkyl, or halogen; R₆ is —NZ₅Z₆; Z₅ is hydrogen; Z₆ is hydrogen, (C₁-C₆)alkyl, (C₁-C₆)alkylcarbonyl. In certain embodiments, the inventive methods include treating dyslipidemia, insulin resistance, β-cell dysfunction, hyperglycemia, metabolic syndrome, and any form of diabetes mellitus including type I and type II diabetes, in a patient comprising administering to the patient in need of such treatment a therapeutically effective amount of a pharmaceutical composition comprising at least one pharmaceutically acceptable carrier and a compound of Formula (I), or a pharmaceutically acceptable salt thereof, wherein R₁ is hydrogen or acetyl; R₂, R₃, R₄, and R₅ are independently hydrogen, halo(C₁-C₆)alkyl, or halogen; R₆ is —NZ₅Z₆; Z₅ is hydrogen; Z₆ is hydrogen, (C₁-C₆)alkyl, (C₁-C₆)alkylcarbonyl.

In certain embodiments, the present invention provides methods for reducing advanced glycated end products and lipid peroxidation including, but not limited to, oxidation of low-density lipoproteins, in a patient comprising administering to the patient in need of such treatment a therapeutically effective amount of a pharmaceutical composition comprising at least one pharmaceutically acceptable carrier and a compound of Formula (I), or a pharmaceutically acceptable salt thereof, wherein R₁ is hydrogen or acetyl; R₂, R₃, R₄, and R₅ are independently hydrogen, halo(C₁-C₆)alkyl, or halogen; R₆ is —NZ₅Z₆; Z₅ is hydrogen; Z₆ is hydrogen, (C₁-C₆)alkyl, (C₁-C₆)alkylcarbonyl.

The present invention additionally provides methods for treating atherosclerosis, neuropathy, nephropathy, retinopathy, inflammatory disorders, cardiovascular diseases, and metabolic disorders in a mammal or patient which includes the step of administering to the mammal or patient in need of such treatment a therapeutically effective amount of a compound of Formula (I), or a pharmaceutically acceptable salt thereof, wherein R₁ is hydrogen or acetyl; R₂, R₃, R₄, and R₅ are independently hydrogen, trifluoromethyl, or Cl; R₆ is —NZ₅Z₆; Z₅ is hydrogen; and Z₆ is hydrogen.

In certain embodiments, the inventive methods include treating dyslipidemia, insulin resistance, β-cell dysfunction, hyperglycemia, metabolic syndrome, and any form of diabetes mellitus including type I and type II diabetes, in a patient comprising administering to the patient in need of such treatment a therapeutically effective amount of a compound of Formula (I), or a pharmaceutically acceptable salt thereof, wherein R₁ is hydrogen or acetyl; R₂, R₃, R₄, and R₅ are independently hydrogen, trifluoromethyl, or Cl; R₆ is —NZ₅Z₆; Z₅ is hydrogen; and Z₆ is hydrogen.

In certain embodiments, the present invention provides methods for reducing advanced glycated end products and/or lipid peroxidation including, but not limited to, oxidation of low-density lipoproteins in a mammal or patient comprising administering to the mammal or patient in need of such treatment a therapeutically effective amount of a compound of Formula (I), or a pharmaceutically acceptable salt thereof, wherein R₁ is hydrogen or acetyl; R₂, R₃, R₄, and R₅ are independently hydrogen, trifluoromethyl, or Cl; R₆ is —NZ₅Z₆; Z₅ is hydrogen; and Z₆ is hydrogen.

In certain embodiments, the inventive methods include treating dyslipidemia, insulin resistance, β-cell dysfunction, hyperglycemia, metabolic syndrome, and any form of diabetes mellitus including type I and type II diabetes, in a patient comprising administering to the patient in need of such treatment a therapeutically effective amount of a pharmaceutical composition comprising at least one pharmaceutically acceptable carrier and a compound of Formula (I), or a pharmaceutically acceptable salt thereof, wherein R₁ is hydrogen or acetyl; R₂, R₃, R₄, and R₅ are independently hydrogen, trifluoromethyl, or Cl; R₆ is —NZ₅Z₆; Z₅ is hydrogen; and Z₆ is hydrogen.

In certain embodiments, the present invention provides methods for reducing advanced glycated end products and lipid peroxidation including, but not limited to, oxidation of low-density lipoproteins, in a patient comprising administering to the patient in need of such treatment a therapeutically effective amount of a pharmaceutical composition comprising at least one pharmaceutically acceptable carrier and a compound of Formula (I), or a pharmaceutically acceptable salt thereof, wherein R₁ is hydrogen or acetyl; R₂, R₃, R₄, and R₅ are independently hydrogen, trifluoromethyl, or Cl; R₆ is —NZ₅Z₆; Z₅ is hydrogen; and Z₆ is hydrogen.

The present invention also provides methods for treating atherosclerosis, neuropathy, nephropathy, retinopathy, inflammatory disorders, cardiovascular diseases, and metabolic disorders in a mammal or patient which includes the step of administering to the mammal or patient in need of such treatment a therapeutically effective amount of a compound of Formula (I), or a pharmaceutically acceptable salt thereof, wherein R₁ is hydrogen or acetyl; R₂, R₃, R₄, and R₅ are independently hydrogen, halo(C₁-C₆)alkyl, or halogen; R₆ is —NZ₅Z₆; Z₅ is hydrogen; Z₆ is phenyl, wherein the phenyl is optionally substituted with 1, 2, 3, 4, or 5 groups that are independently (C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ₇Z₈, or (NZ₇Z₈)carbonyl; and Z₇ and Z₈ are independently hydrogen, (C₁-C₆)alkyl, or (C₁-C₆)alkylcarbonyl.

In certain embodiments, the inventive methods include treating dyslipidemia, insulin resistance, β-cell dysfunction, hyperglycemia, metabolic syndrome, and any form of diabetes mellitus including type I and type II diabetes, in a patient comprising administering to the patient in need of such treatment a therapeutically effective amount of a compound of Formula (I), or a pharmaceutically acceptable salt thereof, wherein R₁ is hydrogen or acetyl; R₂, R₃, R₄, and R₅ are independently hydrogen, halo(C₁-C₆)alkyl, or halogen; R₆ is —NZ₅Z₆; Z₅ is hydrogen; Z₆ is phenyl, wherein the phenyl is optionally substituted with 1, 2, 3, 4, or 5 groups that are independently (C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ₇Z₈, or (NZ₇Z₈)carbonyl; and Z₇ and Z_{g} are independently hydrogen, (C₁-C₆)alkyl, or (C₁-C₆)alkylcarbonyl.

In certain embodiments, the present invention provides methods for reducing advanced glycated end products and/or lipid peroxidation including, but not limited to, oxidation of low-density lipoproteins in a mammal or patient comprising administering to the mammal or patient in need of such treatment a therapeutically effective amount of a compound of Formula (I), or a pharmaceutically acceptable salt thereof, wherein R₁ is hydrogen or acetyl; R₂, R₃, R₄, and R₅ are independently hydrogen, halo(C₁-C₆)alkyl, or halogen; R₆ is —NZ₅Z₆; Z₅ is hydrogen; Z₆ is phenyl, wherein the phenyl is optionally substituted with 1, 2, 3, 4, or 5 groups that are independently (C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ₇Z₈, or (NZ₇Z₈)carbonyl; and Z₇ and Z₈ are independently hydrogen, (C₁-C₆)alkyl, or (C₁-C₆)alkylcarbonyl.

In certain embodiments, the inventive methods include treating dyslipidemia, insulin resistance, β-cell dysfunction, hyperglycemia, metabolic syndrome, and any form of diabetes mellitus including type I and type II diabetes, in a patient comprising administering to the patient in need of such treatment a therapeutically effective amount of a pharmaceutical composition comprising at least one pharmaceutically acceptable carrier and a compound of Formula (I), or a pharmaceutically acceptable salt thereof, wherein R₁ is hydrogen or acetyl; R₂, R₃, R₄, and R₅ are independently hydrogen, halo(C₁-C₆)alkyl, or halogen; R₆ is —NZ₅Z₆; Z₅ is hydrogen; Z₆ is phenyl, wherein the phenyl is optionally substituted with 1, 2, 3, 4, or 5 groups that are independently (C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ₇Z₈, or (NZ₇Z₈)carbonyl; and Z₇ and Z₈ are independently hydrogen, (C₁-C₆)alkyl, or (C₁-C₆)alkylcarbonyl.

In certain embodiments, the present invention provides methods for reducing advanced glycated end products and lipid peroxidation including, but not limited to, oxidation of low-density lipoproteins, in a patient comprising administering to the patient in need of such treatment a therapeutically effective amount of a pharmaceutical composition comprising at least one pharmaceutically acceptable carrier and a compound of Formula (I), or a pharmaceutically acceptable salt thereof, wherein R₁ is hydrogen or acetyl; R₂, R₃, R₄, and R₅ are independently hydrogen, halo(C₁-C₆)alkyl, or halogen; R₆ is —NZ₅Z₆; Z₅ is hydrogen; Z₆ is phenyl, wherein the phenyl is optionally substituted with 1, 2, 3, 4, or 5 groups that are independently (C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ₇Z₈, or (NZ₇Z₈)carbonyl; and Z₇ and Z₈ are independently hydrogen, (C₁-C₆)alkyl, or (C₁-C₆)alkylcarbonyl.

The present invention provides methods for treating atherosclerosis, neuropathy, nephropathy, retinopathy, inflammatory disorders, cardiovascular diseases, and metabolic disorders in a mammal or patient which includes the step of administering to the mammal or patient in need of such treatment a therapeutically effective amount of a compound of Formula (I), or a pharmaceutically acceptable salt thereof, wherein R₁ is hydrogen or acetyl; R₂, R₃, R₄, and R₅ are independently hydrogen, halo(C₁-C₆)alkyl, or halogen; R₆ is —NZ₅Z₆; Z₅ is hydrogen; Z₆ is phenyl, wherein the phenyl is optionally substituted with 1 or 2 groups that are independently halo(C₁-C₆)alkyl or halogen.

In certain embodiments, the inventive methods include treating dyslipidemia, insulin resistance, β-cell dysfunction, hyperglycemia, metabolic syndrome, and any form of diabetes mellitus including type I and type II diabetes, in a patient comprising administering to the patient in need of such treatment a therapeutically effective amount of a compound of Formula (I), or a pharmaceutically acceptable salt thereof, wherein R₁ is hydrogen or acetyl; R₂, R₃, R₄, and R₅ are independently hydrogen, halo(C₁-C₆)alkyl, or halogen; R₆ is —NZ₅Z₆; Z₅ is hydrogen; Z₆ is phenyl, wherein the phenyl is optionally substituted with 1 or 2 groups that are independently halo(C₁-C₆)alkyl or halogen.

In certain embodiments, the present invention provides methods for reducing advanced glycated end products and/or lipid peroxidation including, but not limited to, oxidation of low-density lipoproteins in a mammal or patient comprising administering to the mammal or patient in need of such treatment a therapeutically effective amount of a compound of Formula (I), or a pharmaceutically acceptable salt thereof, wherein R₁ is hydrogen or acetyl; R₂, R₃, R₄, and R₅ are independently hydrogen, halo(C₁-C₆)alkyl, or halogen; R₆ is —NZ₅Z₆; Z₅ is hydrogen; Z₆ is phenyl, wherein the phenyl is optionally substituted with 1 or 2 groups that are independently halo(C₁-C₆)alkyl or halogen.

In certain embodiments, the inventive methods include treating dyslipidemia, insulin resistance, β-cell dysfunction, hyperglycemia, metabolic syndrome, and any form of diabetes mellitus including type I and type II diabetes, in a patient comprising administering to the patient in need of such treatment a therapeutically effective amount of a pharmaceutical composition comprising at least one pharmaceutically acceptable carrier and a compound of Formula (I), or a pharmaceutically acceptable salt thereof, wherein R₁ is hydrogen or acetyl; R₂, R₃, R₄, and R₅ are independently hydrogen, halo(C₁-C₆)alkyl, or halogen; R₆ is —NZ₅Z₆; Z₅ is hydrogen; Z₆ is phenyl, wherein the phenyl is optionally substituted with 1 or 2 groups that are independently halo(C₁-C₆)alkyl or halogen.

In certain embodiments, the present invention provides methods for reducing advanced glycated end products and lipid peroxidation including, but not limited to, oxidation of low-density lipoproteins, in a patient comprising administering to the patient in need of such treatment a therapeutically effective amount of a pharmaceutical composition comprising at least one pharmaceutically acceptable carrier and a compound of Formula (I), or a pharmaceutically acceptable salt thereof, wherein R₁ is hydrogen or acetyl; R₂, R₃, R₄, and R₅ are independently hydrogen, halo(C₁-C₆)alkyl, or halogen; R₆ is —NZ₅Z₆; Z₅ is hydrogen; Z₆ is phenyl, wherein the phenyl is optionally substituted with 1 or 2 groups that are independently halo(C₁-C₆)alkyl or halogen.

In accordance with the present invention, methods are provided for treating atherosclerosis, neuropathy, nephropathy, retinopathy, inflammatory disorders, cardiovascular diseases, and metabolic disorders in a mammal or patient which includes the step of administering to the mammal or patient in need of such treatment a therapeutically effective amount of a compound of Formula (I), or a pharmaceutically acceptable salt thereof, wherein R₁ is hydrogen or acetyl; R₂, R₃, R₄, and R₅ are independently hydrogen, trifluoromethyl, or Cl; R₆ is —NZ₅Z₆; Z₅ is hydrogen; Z₆ is phenyl, wherein the phenyl is optionally substituted with 1 or 2 groups that are independently trifluoromethyll or Cl.

In certain embodiments, the inventive methods include treating dyslipidemia, insulin resistance, β-cell dysfunction, hyperglycemia, metabolic syndrome, and any form of diabetes mellitus including type I and type II diabetes, in a patient comprising administering to the patient in need of such treatment a therapeutically effective amount of a compound of Formula (I), or a pharmaceutically acceptable salt thereof, wherein R₁ is hydrogen or acetyl; R₂, R₃, R₄, and R₅ are independently hydrogen, trifluoromethyl, or Cl; R₆ is —NZ₅Z₆; Z₅ is hydrogen; Z₆ is phenyl, wherein the phenyl is optionally substituted with 1 or 2 groups that are independently trifluoromethyll or Cl.

In certain embodiments, the present invention provides methods for reducing advanced glycated end products and/or lipid peroxidation including, but not limited to, oxidation of low-density lipoproteins in a mammal or patient comprising administering to the mammal or patient in need of such treatment a therapeutically effective amount of a compound of Formula (I), or a pharmaceutically acceptable salt thereof, wherein R₁ is hydrogen or acetyl; R₂, R₃, R₄, and R₅ are independently hydrogen, trifluoromethyl, or Cl; R₆ is —NZ₅Z₆; Z₅ is hydrogen; Z₆ is phenyl, wherein the phenyl is optionally substituted with 1 or 2 groups that are independently trifluoromethyll or Cl.

In certain embodiments, the inventive methods include treating dyslipidemia, insulin resistance, β-cell dysfunction, hyperglycemia, metabolic syndrome, and any form of diabetes mellitus including type I and type II diabetes, in a patient comprising administering to the patient in need of such treatment a therapeutically effective amount of a pharmaceutical composition comprising at least one pharmaceutically acceptable carrier and a compound of Formula (I), or a pharmaceutically acceptable salt thereof, wherein R₁ is hydrogen or acetyl; R₂, R₃, R₄, and R₅ are independently hydrogen, trifluoromethyl, or Cl; R₆ is —NZ₅Z₆; Z₅ is hydrogen; Z₆ is phenyl, wherein the phenyl is optionally substituted with 1 or 2 groups that are independently trifluoromethyll or Cl.

In certain embodiments, the present invention provides methods for reducing advanced glycated end products and lipid peroxidation including, but not limited to, oxidation of low-density lipoproteins, in a patient comprising administering to the patient in need of such treatment a therapeutically effective amount of a pharmaceutical composition comprising at least one pharmaceutically acceptable carrier and a compound of Formula (I), or a pharmaceutically acceptable salt thereof, wherein R₁ is hydrogen or acetyl; R₂, R₃, R₄, and R₅ are independently hydrogen, trifluoromethyl, or Cl; R₆ is —NZ₅Z₆; Z₅ is hydrogen; Z₆ is phenyl, wherein the phenyl is optionally substituted with 1 or 2 groups that are independently trifluoromethyll or Cl.

In accordance with the present invention, methods are provided for treating atherosclerosis, neuropathy, nephropathy, retinopathy, inflammatory disorders, cardiovascular diseases, and metabolic disorders in a mammal or patient which includes the step of administering to the mammal or patient in need of such treatment a therapeutically effective amount of a compound of Formula (I) or Formula (IV), or a pharmaceutically acceptable salt thereof, wherein the compound of Formula (I) or Formula (IV) is N-(3,5-bis(trifluoromethyl)phenyl)-5-chloro-2-hydroxybenzamide or 2-(3,5-bis(trifluoromethyl)phenylcarbamoyl)-4-chlorophenyl acetate.

In certain embodiments, the inventive methods include treating dyslipidemia, insulin resistance, β-cell dysfunction, hyperglycemia, metabolic syndrome, and any form of diabetes mellitus including type I and type II diabetes, in a patient comprising administering to the patient in need of such treatment a therapeutically effective amount of a compound of Formula (I) or Formula (IV), or a pharmaceutically acceptable salt thereof, wherein the compound of Formula (I) or Formula (IV) is N-(3,5-bis(trifluoromethyl)phenyl)-5-chloro-2-hydroxybenzamide or 2-(3,5-bis(trifluoromethyl)phenylcarbamoyl)-4-chlorophenyl acetate.

In certain embodiments, the present invention provides methods for reducing advanced glycated end products and/or lipid peroxidation including, but not limited to, oxidation of low-density lipoproteins in a mammal or patient comprising administering to the mammal or patient in need of such treatment a therapeutically effective amount of a compound of Formula (I) or Formula (IV), or a pharmaceutically acceptable salt thereof, wherein the compound of Formula (I) or Formula (IV) is N-(3,5-bis(trifluoromethyl)phenyl)-5-chloro-2-hydroxybenzamide or 2-(3,5-bis(trifluoromethyl)phenylcarbamoyl)-4-chlorophenyl acetate.

In certain embodiments, the inventive methods include treating dyslipidemia, insulin resistance, β-cell dysfunction, hyperglycemia, metabolic syndrome, and any form of diabetes mellitus including type I and type II diabetes, in a patient comprising administering to the patient in need of such treatment a therapeutically effective amount of a pharmaceutical composition comprising at least one pharmaceutically acceptable carrier and a compound of Formula (I) or Formula (IV), or a pharmaceutically acceptable salt thereof, wherein the compound of Formula (I) or Formula (IV) is N-(3,5-bis(trifluoromethyl)phenyl)-5-chloro-2-hydroxybenzamide or 2-(3,5-bis(trifluoromethyl)phenylcarbamoyl)-4-chlorophenyl acetate.

In certain embodiments, the present invention provides methods for reducing advanced glycated end products and lipid peroxidation including, but not limited to, oxidation of low-density lipoproteins, in a patient comprising administering to the patient in need of such treatment a therapeutically effective amount of a pharmaceutical composition comprising at least one pharmaceutically acceptable carrier and a compound of Formula (I) or Formula (IV), or a pharmaceutically acceptable salt thereof, wherein the compound of Formula (I) or Formula (IV) is N-(3,5-bis(trifluoromethyl)phenyl)-5-chloro-2-hydroxybenzamide or 2-(3,5-bis(trifluoromethyl)phenylcarbamoyl)-4-chlorophenyl acetate.

In another aspect, the present invention provides methods for treating atherosclerosis, neuropathy, nephropathy, retinopathy, inflammatory disorders, cardiovascular diseases, and metabolic disorders in a mammal or patient that comprises administering to the mammal or patient in need of such treatment a therapeutically effective amount of a compound selected from Example 1, 2, 3, 4, 5, 6, 7, 8, 9, 10, 11, 12, 13, 14, 15, 16, 17, 18, 19, 20, or 21.

In certain embodiments, the inventive methods include treating dyslipidemia, insulin resistance, β-cell dysfunction, hyperglycemia, metabolic syndrome, and any form of diabetes mellitus including type I and type II diabetes, in a patient comprising administering to the patient in need of such treatment a therapeutically effective amount of a compound selected from Example 1, 2, 3, 4, 5, 6, 7, 8, 9, 10, 11, 12, 13, 14, 15, 16, 17, 18, 19, 20, or 21.

In certain embodiments, the present invention provides methods for reducing advanced glycated end products and/or lipid peroxidation including, but not limited to, oxidation of low-density lipoproteins in a mammal or patient comprising administering to the mammal or patient in need of such treatment a therapeutically effective amount of a compound selected from Example 1, 2, 3, 4, 5, 6, 7, 8, 9, 10, 11, 12, 13, 14, 15, 16, 17, or 18, 19, 20, or 21.

In another aspect, the present invention provides methods for treating hyperglycemia in a mammal or patient that comprises administering to the mammal or patient in need of such treatment a therapeutically effective amount of a compound selected from Example 1, 2, 3, 4, 5, 6, 7, 8, 9, 10, 11, 12, 13, 14, 15, 16, 17, or 18, 19, 20, or 21.

In another aspect, the present invention provides methods for reducing triglycerides and/or free fatty acids in a mammal or patient that comprises administering to the mammal or patient in need of such treatment a therapeutically effective amount of a compound selected from Example 1, 2, 3, 4, 5, 6, 7, 8, 9, 10, 11, 12, 13, 14, 15, 16, 17, 18, 19, 20, or 21.

In another aspect, the present invention provides methods for treating β-cell dysfunction in a mammal or patient that comprises administering to the mammal or patient in need of such treatment a therapeutically effective amount of a compound selected from Example 1, 2, 3, 4, 5, 6, 7, 8, 9, 10, 11, 12, 13, 14, 15, 16, 17, 18, 19, 20, or 21.

In certain embodiments, the inventive methods include treating dyslipidemia, insulin resistance, β-cell dysfunction, hyperglycemia, metabolic syndrome, and any form of diabetes mellitus including type I and type II diabetes, in a patient comprising administering to the patient in need of such treatment a pharmaceutical composition comprising at least one pharmaceutically acceptable carrier and a compound selected from Example 1, 2, 3, 4, 5, 6, 7, 8, 9, 10, 11, 12, 13, 14, 15, 16, 17, 18, 19, 20, or 21.

In certain embodiments, the present invention provides methods for reducing advanced glycated end products and lipid peroxidation including, but not limited to, oxidation of low-density lipoproteins, in a patient comprising administering to the patient in need of such treatment a therapeutically effective amount of a pharmaceutical composition comprising at least one pharmaceutically acceptable carrier and a compound selected from Example 1, 2, 3, 4, 5, 6, 7, 8, 9, 10, 11, 12, 13, 14, 15, 16, 17, 18, 19, 20, or 21.

In another aspect, the present invention provides methods for treating atherosclerosis, neuropathy, nephropathy, retinopathy, inflammatory disorders, cardiovascular diseases, and metabolic disorders in a mammal or patient that comprises administering to the mammal or patient in need of such treatment a therapeutically effective amount of Example 1 (salnacedin).

In certain embodiments, the inventive methods include treating dyslipidemia, insulin resistance, β-cell dysfunction, hyperglycemia, metabolic syndrome, and any form of diabetes mellitus including type I and type II diabetes, in a patient comprising administering to the patient in need of such treatment a therapeutically effective amount of Example 1.

In certain embodiments, the present invention provides methods for reducing advanced glycated end products and/or lipid peroxidation including, but not limited to, oxidation of low-density lipoproteins in a mammal or patient comprising administering to the mammal or patient in need of such treatment a therapeutically effective amount of Example 1.

In certain embodiments, the present invention provides methods for reducing triglycerides and/or free fatty acids in a mammal or patient comprising administering to the mammal or patient in need of such treatment a therapeutically effective amount of Example 1.

In certain embodiments, the inventive methods include treating dyslipidemia, insulin resistance, β-cell dysfunction, hyperglycemia, metabolic syndrome, and any form of diabetes mellitus including type I and type II diabetes, in a patient comprising administering to the patient in need of such treatment a therapeutically effective amount of a pharmaceutical composition comprising at least one pharmaceutically acceptable carrier and Example 1.

In certain embodiments, the present invention provides methods for reducing advanced glycated end products and lipid peroxidation including, but not limited to, oxidation of low-density lipoproteins, in a patient comprising administering to the patient in need of such treatment a therapeutically effective amount of a pharmaceutical composition comprising administering to the patient in need of such treatment a therapeutically effective amount of a pharmaceutical composition comprising at least one pharmaceutically acceptable carrier and Example 1.

In certain embodiments, the present invention provides methods for reducing triglycerides and/or free fatty acids, in a patient comprising administering to the patient in need of such treatment a therapeutically effective amount of a pharmaceutical composition comprising administering to the patient in need of such treatment a therapeutically effective amount of a pharmaceutical composition comprising at least one pharmaceutically acceptable carrier and Example 1.

In another aspect, the present invention provides methods for treating atherosclerosis, neuropathy, nephropathy, retinopathy, inflammatory disorders, cardiovascular diseases, and metabolic disorders in a mammal or patient that comprises administering to the mammal or patient in need of such treatment a therapeutically effective amount of Example 4.

In certain embodiments, the inventive methods include treating dyslipidemia, insulin resistance, β-cell dysfunction, hyperglycemia, metabolic syndrome, and any form of diabetes mellitus including type I and type II diabetes, in a patient comprising administering to the patient in need of such treatment a therapeutically effective amount of Example 4.

In certain embodiments, the present invention provides methods for reducing advanced glycated end products and/or lipid peroxidation including, but not limited to, oxidation of low-density lipoproteins in a mammal or patient comprising administering to the mammal or patient in need of such treatment a therapeutically effective amount of Example 4.

In certain embodiments, the inventive methods include treating dyslipidemia, insulin resistance, β-cell dysfunction, hyperglycemia, metabolic syndrome, and any form of diabetes mellitus including type I and type II diabetes, in a patient comprising administering to the patient in need of such treatment a therapeutically effective amount of a pharmaceutical composition comprising at least one pharmaceutically acceptable carrier and Example 4.

In certain embodiments, the present invention provides methods for reducing advanced glycated end products and lipid peroxidation including, but not limited to, oxidation of low-density lipoproteins, in a patient comprising administering to the patient in need of such treatment a therapeutically effective amount of a pharmaceutical composition comprising administering to the patient in need of such treatment a therapeutically effective amount of a pharmaceutical composition comprising at least one pharmaceutically acceptable carrier and Example 4.

In another aspect, the present invention provides methods for treating atherosclerosis, neuropathy, nephropathy, retinopathy, inflammatory disorders, cardiovascular diseases, and metabolic disorders in a mammal or patient that comprises administering to the mammal or patient in need of such treatment a therapeutically effective amount of Example 7. In certain embodiments, the inventive methods include treating dyslipidemia, insulin resistance, β-cell dysfunction, hyperglycemia, metabolic syndrome, and any form of diabetes mellitus including type I and type II diabetes, in a patient comprising administering to the patient in need of such treatment a therapeutically effective amount of Example 7.

In certain embodiments, the present invention provides methods for reducing advanced glycated end products and/or lipid peroxidation including, but not limited to, oxidation of low-density lipoproteins in a mammal or patient comprising administering to the mammal or patient in need of such treatment a therapeutically effective amount of Example 7.

In certain embodiments, the inventive methods include treating dyslipidemia, insulin resistance, β-cell dysfunction, hyperglycemia, metabolic syndrome, and any form of diabetes mellitus including type I and type II diabetes, in a patient comprising administering to the patient in need of such treatment a therapeutically effective amount of a pharmaceutical composition comprising at least one pharmaceutically acceptable carrier and Example 7.

In certain embodiments, the present invention provides methods for reducing advanced glycated end products and lipid peroxidation including, but not limited to, oxidation of low-density lipoproteins, in a patient comprising administering to the patient in need of such treatment a therapeutically effective amount of a pharmaceutical composition comprising administering to the patient in need of such treatment a therapeutically effective amount of a pharmaceutical composition comprising at least one pharmaceutically acceptable carrier and Example 7.

In another aspect, the present invention provides methods for treating atherosclerosis, neuropathy, nephropathy, retinopathy, inflammatory disorders, cardiovascular diseases, and metabolic disorders in a mammal or patient that comprises administering to the mammal or patient in need of such treatment a therapeutically effective amount of Example 10.

In certain embodiments, the inventive methods include treating dyslipidemia, insulin resistance, β-cell dysfunction, hyperglycemia, metabolic syndrome, and any form of diabetes mellitus including type I and type II diabetes, in a patient comprising administering to the patient in need of such treatment a therapeutically effective amount of Example 10.

In certain embodiments, the present invention provides methods for reducing advanced glycated end products and/or lipid peroxidation including, but not limited to, oxidation of low-density lipoproteins in a mammal or patient comprising administering to the mammal or patient in need of such treatment a therapeutically effective amount of Example 10.

In certain embodiments, the inventive methods include treating dyslipidemia, insulin resistance, β-cell dysfunction, hyperglycemia, metabolic syndrome, and any form of diabetes mellitus including type I and type II diabetes, in a patient comprising administering to the patient in need of such treatment a therapeutically effective amount of a pharmaceutical composition comprising at least one pharmaceutically acceptable carrier and Example 10.

In certain embodiments, the present invention provides methods for reducing advanced glycated end products and lipid peroxidation including, but not limited to, oxidation of low-density lipoproteins, in a patient comprising administering to the patient in need of such treatment a therapeutically effective amount of a pharmaceutical composition comprising administering to the patient in need of such treatment a therapeutically effective amount of a pharmaceutical composition comprising at least one pharmaceutically acceptable carrier and Example 10.

In another aspect, the present invention provides methods for treating atherosclerosis, neuropathy, nephropathy, retinopathy, inflammatory disorders, cardiovascular diseases, and metabolic disorders in a mammal or patient that comprises administering to the mammal or patient in need of such treatment a therapeutically effective amount of Example 13.

In certain embodiments, the inventive methods include treating dyslipidemia, insulin resistance, β-cell dysfunction, hyperglycemia, metabolic syndrome, and any form of diabetes mellitus including type I and type II diabetes, in a patient comprising administering to the patient in need of such treatment a therapeutically effective amount of Example 13.

In certain embodiments, the present invention provides methods for reducing advanced glycated end products and/or lipid peroxidation including, but not limited to, oxidation of low-density lipoproteins in a mammal or patient comprising administering to the mammal or patient in need of such treatment a therapeutically effective amount of Example 13.

In certain embodiments, the present invention provides methods for reducing triglycerides and/or free fatty acids in a mammal or patient comprising administering to the mammal or patient in need of such treatment a therapeutically effective amount of Example 13.

In certain embodiments, the inventive methods include treating dyslipidemia, insulin resistance, β-cell dysfunction, hyperglycemia, metabolic syndrome, and any form of diabetes mellitus including type I and type II diabetes, in a patient comprising administering to the patient in need of such treatment a therapeutically effective amount of a pharmaceutical composition comprising at least one pharmaceutically acceptable carrier and Example 13.

In certain embodiments, the present invention provides methods for reducing advanced glycated end products and lipid peroxidation including, but not limited to, oxidation of low-density lipoproteins, in a patient comprising administering to the patient in need of such treatment a therapeutically effective amount of a pharmaceutical composition comprising administering to the patient in need of such treatment a therapeutically effective amount of a pharmaceutical composition comprising at least one pharmaceutically acceptable carrier and Example 13.

In certain embodiments, the present invention provides methods for reducing triglycerides and/or free fatty acids, in a patient comprising administering to the patient in need of such treatment a therapeutically effective amount of a pharmaceutical composition comprising administering to the patient in need of such treatment a therapeutically effective amount of a pharmaceutical composition comprising at least one pharmaceutically acceptable carrier and Example 13.

In another aspect, the present invention provides methods for treating atherosclerosis, neuropathy, nephropathy, retinopathy, inflammatory disorders, cardiovascular diseases, and metabolic disorders in a mammal or patient that comprises administering to the mammal or patient in need of such treatment a therapeutically effective amount of Example 16.

In certain embodiments, the inventive methods include treating dyslipidemia, insulin resistance, β-cell dysfunction, hyperglycemia, metabolic syndrome, and any form of diabetes mellitus including type I and type II diabetes, in a patient comprising administering to the patient in need of such treatment a therapeutically effective amount of Example 16.

In certain embodiments, the present invention provides methods for reducing advanced glycated end products and/or lipid peroxidation including, but not limited to, oxidation of low-density lipoproteins in a mammal or patient comprising administering to the mammal or patient in need of such treatment a therapeutically effective amount of Example 16.

In certain embodiments, the inventive methods include treating dyslipidemia, insulin resistance, β-cell dysfunction, hyperglycemia, metabolic syndrome, and any form of diabetes mellitus including type I and type II diabetes, in a patient comprising administering to the patient in need of such treatment a therapeutically effective amount of a pharmaceutical composition comprising at least one pharmaceutically acceptable carrier and Example 16.

In certain embodiments, the present invention provides methods for reducing advanced glycated end products and lipid peroxidation including, but not limited to, oxidation of low-density lipoproteins, in a patient comprising administering to the patient in need of such treatment a therapeutically effective amount of a pharmaceutical composition comprising administering to the patient in need of such treatment a therapeutically effective amount of a pharmaceutical composition comprising at least one pharmaceutically acceptable carrier and Example 16.

In another aspect, the present invention provides methods for treating atherosclerosis, neuropathy, nephropathy, retinopathy, inflammatory disorders, cardiovascular diseases, and metabolic disorders in a mammal or patient that comprises administering to the mammal or patient in need of such treatment a therapeutically effective amount of one of Example 19, 20, or 21.

In certain embodiments, the inventive methods include treating dyslipidemia, insulin resistance, β-cell dysfunction, hyperglycemia, metabolic syndrome, and any form of diabetes mellitus including type I and type II diabetes, in a patient comprising administering to the patient in need of such treatment a therapeutically effective amount of one of Example 19, 20, or 21.

In certain embodiments, the present invention provides methods for reducing advanced glycated end products and/or lipid peroxidation including, but not limited to, oxidation of low-density lipoproteins in a mammal or patient comprising administering to the mammal or patient in need of such treatment a therapeutically effective amount of one of Example 19, 20, or 21.

In certain embodiments, the present invention provides methods for reducing triglycerides and/or free fatty acids in a mammal or patient comprising administering to the mammal or patient in need of such treatment a therapeutically effective amount of one of Example 19, 20, or 21.

In certain embodiments, the inventive methods include treating dyslipidemia, insulin resistance, β-cell dysfunction, hyperglycemia, metabolic syndrome, and any form of diabetes mellitus including type I and type II diabetes, in a patient comprising administering to the patient in need of such treatment a therapeutically effective amount of a pharmaceutical composition comprising at least one pharmaceutically acceptable carrier and one of Example 19, 20, or 21.

In certain embodiments, the present invention provides methods for reducing advanced glycated end products and lipid peroxidation including, but not limited to, oxidation of low-density lipoproteins, in a patient comprising administering to the patient in need of such treatment a therapeutically effective amount of a pharmaceutical composition comprising administering to the patient in need of such treatment a therapeutically effective amount of a pharmaceutical composition comprising at least one pharmaceutically acceptable carrier and one of Example 19, 20, or 21.

In certain embodiments, the present invention provides methods for reducing triglycerides and/or free fatty acids, in a patient comprising administering to the patient in need of such treatment a therapeutically effective amount of a pharmaceutical composition comprising administering to the patient in need of such treatment a therapeutically effective amount of a pharmaceutical composition comprising at least one pharmaceutically acceptable carrier and one of Example 19, 20, or 21.

In another aspect, the present invention provides uses for compounds of Formula (I) for preparing, or for the manufacture of, a medicament for treating atherosclerosis, neuropathy, nephropathy, retinopathy, inflammatory disorders, cardiovascular diseases, and metabolic disorders in a mammal or patient, wherein R₁ is hydrogen or acetyl; R₂, R₃, R₄, and R₅ are independently hydrogen, trifluoromethyl, or 2,4-difluorophenyl; R₆ is formula (i); and R₇, R₈, R₉, X₁, and L are as defined in Formula (I) of the Summary section.

In certain embodiments, the present invention provides uses for compounds of Formula (I) for preparing, or for the manufacture of, a medicament for treating dyslipidemia, insulin resistance, β-cell dysfunction, hyperglycemia, metabolic syndrome, and any form of diabetes mellitus including type I and type II diabetes, in a patient, wherein R₁ is hydrogen or acetyl; R₂, R₃, R₄, and R₅ are independently hydrogen, trifluoromethyl, or 2,4-difluorophenyl; R₆ is formula (i); and R₇, R₈, R₉, X₁, and L are as defined in Formula (I) of the Summary section.

In certain embodiments, the present invention provides uses for compounds of Formula (I) for preparing, or for the manufacture of, a medicament for reducing advanced glycated end products and/or lipid peroxidation including, but not limited to, oxidation of low-density lipoproteins in a patient, wherein R₁ is hydrogen or acetyl; R₂, R₃, R₄, and R₅ are independently hydrogen, trifluoromethyl, or 2,4-difluorophenyl; R₆ is formula (i); and R₇, R₈, R₉, X₁, and L are as defined in Formula (I) of the Summary section.

In certain embodiments, the present invention provides uses for pharmaceutical compositions for preparing, or for the manufacture of, a medicament for treating dyslipidemia, insulin resistance, β-cell dysfunction, hyperglycemia, metabolic syndrome, and any form of diabetes mellitus including type I and type II diabetes, in a patient, wherein the pharmaceutical composition comprises at least one pharmaceutically acceptable carrier and a compound of Formula (I), or a pharmaceutically acceptable salt thereof, wherein R₁ is hydrogen or acetyl; R₂, R₃, R₄, and R₅ are independently hydrogen, trifluoromethyl, or 2,4-difluorophenyl; R₆ is formula (i); and R₇, R₈, R₉, X₁, and L are as defined in Formula (I) of the Summary section.

In certain embodiments, the present invention provides uses for pharmaceutical compositions for preparing, or for the manufacture of, a medicament for reducing advanced glycated end products and/or lipid peroxidation including, but not limited to, oxidation of low-density lipoproteins in a patient, wherein the pharmaceutical composition comprises at least one pharmaceutically acceptable carrier and a compound of Formula (I), or a pharmaceutically acceptable salt thereof, wherein R₁ is hydrogen or acetyl; R₂, R₃, R₄, and R₅ are independently hydrogen, trifluoromethyl, or 2,4-difluorophenyl; R₆ is formula (i); and R₇, R₈, R₉, X₁, and L are as defined in Formula (I) of the Summary section.

In another aspect, the present invention provides uses for compounds of Formula (I) for preparing, or for the manufacture of, a medicament for treating atherosclerosis, neuropathy, nephropathy, retinopathy, inflammatory disorders, cardiovascular diseases, and metabolic disorders in a mammal or patient, wherein R₁ is hydrogen or acetyl; R₂, R₃, R₄, and R₅ are independently hydrogen, trifluoromethyl, or 2,4-difluorophenyl; R₆ is formula (i); R₇ is (C₁-C₆)alkoxy or hydroxy; R₉ is hydrogen; R₉ is (C₁-C₆)alkylcarbonyl; X₁ is S; and L is CH₂.

In certain embodiments, the present invention provides uses for compounds of Formula (I) for preparing, or for the manufacture of, a medicament for treating dyslipidemia, insulin resistance, β-cell dysfunction, hyperglycemia, metabolic syndrome, and any form of diabetes mellitus including type I and type II diabetes, in a patient, wherein R₁ is hydrogen or acetyl; R₂, R₃, R₄, and R₅ are independently hydrogen, trifluoromethyl, or 2,4-difluorophenyl; R₆ is formula (i); R₇ is (C₁-C₆)alkoxy or hydroxy; R₉ is hydrogen; R₉ is (C₁-C₆)alkylcarbonyl; X₁ is S; and L is CH₂.

In certain embodiments, the present invention provides uses for compounds of Formula (I) for preparing, or for the manufacture of, a medicament for reducing advanced glycated end products and/or lipid peroxidation including, but not limited to, oxidation of low-density lipoproteins in a patient, wherein R₁ is hydrogen or acetyl; R₂, R₃, R₄, and R₅ are independently hydrogen, trifluoromethyl, or 2,4-difluorophenyl; R₆ is formula (i); R₇ is (C₁-C₆)alkoxy or hydroxy; R₉ is hydrogen; R₉ is (C₁-C₆)alkylcarbonyl; X₁ is S; and L is CH₂.

In certain embodiments, the present invention provides uses for pharmaceutical compositions for preparing, or for the manufacture of, a medicament for treating dyslipidemia, insulin resistance, β-cell dysfunction, hyperglycemia, metabolic syndrome, and any form of diabetes mellitus including type I and type II diabetes, in a patient, wherein the pharmaceutical composition comprises at least one pharmaceutically acceptable carrier and a compound of Formula (I), or a pharmaceutically acceptable salt thereof, wherein R₁ is hydrogen or acetyl; R₂, R₃, R₄, and R₅ are independently hydrogen, trifluoromethyl, or 2,4-difluorophenyl; R₆ is formula (i); R₇ is (C₁-C₆)alkoxy or hydroxy; R₉ is hydrogen; R₉ is (C₁-C₆)alkylcarbonyl; X₁ is S; and L is CH₂.

In certain embodiments, the present invention provides uses for pharmaceutical compositions for preparing, or for the manufacture of, a medicament for reducing advanced glycated end products and/or lipid peroxidation including, but not limited to, oxidation of low-density lipoproteins in a patient, wherein the pharmaceutical composition comprises at least one pharmaceutically acceptable carrier and a compound of Formula (I), or a pharmaceutically acceptable salt thereof, wherein R₁ is hydrogen or acetyl; R₂, R₃, R₄, and R₅ are independently hydrogen, trifluoromethyl, or 2,4-difluorophenyl; R₆ is formula (i); R₇ is (C₁-C₆)alkoxy or hydroxy; R₉ is hydrogen; R₉ is (C₁-C₆)alkylcarbonyl; X₁ is S; and L is CH₂.

In another aspect, the present invention provides uses for compounds of Formula (I) for preparing, or for the manufacture of, a medicament for treating atherosclerosis, neuropathy, nephropathy, retinopathy, inflammatory disorders, cardiovascular diseases, and metabolic disorders in a mammal or patient, wherein R₁ is hydrogen or acetyl; R₂, R₃, R₄, and R₅ are independently hydrogen, trifluoromethyl, or 2,4-difluorophenyl; R₆ is formula (i); R₇ is ethoxy, methoxy, or hydroxy; R₉ is hydrogen; R₉ is acetyl; X₁ is S; and L is CH₂.

In certain embodiments, the present invention provides uses for compounds of Formula (I) for preparing, or for the manufacture of, a medicament for treating dyslipidemia, insulin resistance, β-cell dysfunction, hyperglycemia, metabolic syndrome, and any form of diabetes mellitus including type I and type II diabetes, in a patient, wherein R₁ is hydrogen or acetyl; R₂, R₃, R₄, and R₅ are independently hydrogen, trifluoromethyl, or 2,4-difluorophenyl; R₆ is formula (i); R₇ is ethoxy, methoxy, or hydroxy; R₉ is hydrogen; R₉ is acetyl; X₁ is S; and L is CH₂.

In certain embodiments, the present invention provides uses for compounds of Formula (I) for preparing, or for the manufacture of, a medicament for reducing advanced glycated end products and/or lipid peroxidation including, but not limited to, oxidation of low-density lipoproteins in a patient, wherein R₁ is hydrogen or acetyl; R₂, R₃, R₄, and R₅ are independently hydrogen, trifluoromethyl, or 2,4-difluorophenyl; R₆ is formula (i); R₇ is ethoxy, methoxy, or hydroxy; R₉ is hydrogen; R₉ is acetyl; X₁ is S; and L is CH₂.

In certain embodiments, the present invention provides uses for pharmaceutical compositions for preparing, or for the manufacture of, a medicament for treating dyslipidemia, insulin resistance, β-cell dysfunction, hyperglycemia, metabolic syndrome, and any form of diabetes mellitus including type I and type II diabetes, in a patient, wherein the pharmaceutical composition comprises at least one pharmaceutically acceptable carrier and a compound of Formula (I), or a pharmaceutically acceptable salt thereof, wherein R₁ is hydrogen or acetyl; R₂, R₃, R₄, and R₅ are independently hydrogen, trifluoromethyl, or 2,4-difluorophenyl; R₆ is formula (i); R₇ is ethoxy, methoxy, or hydroxy; R₉ is hydrogen; R₉ is acetyl; X₁ is S; and L is CH₂.

In certain embodiments, the present invention provides uses for pharmaceutical compositions for preparing, or for the manufacture of, a medicament for reducing advanced glycated end products and/or lipid peroxidation including, but not limited to, oxidation of low-density lipoproteins in a patient, wherein the pharmaceutical composition comprises at least one pharmaceutically acceptable carrier and a compound of Formula (I), or a pharmaceutically acceptable salt thereof, wherein R₁ is hydrogen or acetyl; R₂, R₃, R₄, and R₅ are independently hydrogen, trifluoromethyl, or 2,4-difluorophenyl; R₆ is formula (i); R₇ is ethoxy, methoxy, or hydroxy; R₉ is hydrogen; R₉ is acetyl; X₁ is S; and L is CH₂.

In another aspect, the present invention provides uses for compounds of Formula (I) for preparing, or for the manufacture of, a medicament for treating atherosclerosis, neuropathy, nephropathy, retinopathy, inflammatory disorders, cardiovascular diseases, and metabolic disorders in a mammal or patient, wherein R₁ is hydrogen or acetyl; R₂, R₃, R₄, and R₅ are independently hydrogen, trifluoromethyl, or 2,4-difluorophenyl; and R₆ is (L) N-acetylcysteine.

In certain embodiments, the present invention provides uses for compounds of Formula (I) for preparing, or for the manufacture of, a medicament for treating dyslipidemia, insulin resistance, β-cell dysfunction, hyperglycemia, metabolic syndrome, and any form of diabetes mellitus including type I and type II diabetes, in a patient, wherein R₁ is hydrogen or acetyl; R₂, R₃, R₄, and R₅ are independently hydrogen, trifluoromethyl, or 2,4-difluorophenyl; and R₆ is (L) N-acetylcysteine.

In certain embodiments, the present invention provides uses for compounds of Formula (I) for preparing, or for the manufacture of, a medicament for reducing advanced glycated end products and/or lipid peroxidation including, but not limited to, oxidation of low-density lipoproteins in a patient, wherein R₁ is hydrogen or acetyl; R₂, R₃, R₄, and R₅ are independently hydrogen, trifluoromethyl, or 2,4-difluorophenyl; and R₆ is (L) N-acetylcysteine.

In certain embodiments, the present invention provides uses for pharmaceutical compositions for preparing, or for the manufacture of, a medicament for treating dyslipidemia, insulin resistance, β-cell dysfunction, hyperglycemia, metabolic syndrome, and any form of diabetes mellitus including type I and type II diabetes, in a patient, wherein the pharmaceutical composition comprises at least one pharmaceutically acceptable carrier and a compound of Formula (I), or a pharmaceutically acceptable salt thereof, wherein R₁ is hydrogen or acetyl; R₂, R₃, R₄, and R₅ are independently hydrogen, trifluoromethyl, or 2,4-difluorophenyl; and R₆ is (L) N-acetylcysteine.

In certain embodiments, the present invention provides uses for pharmaceutical compositions for preparing, or for the manufacture of, a medicament for reducing advanced glycated end products and/or lipid peroxidation including, but not limited to, oxidation of low-density lipoproteins in a patient, wherein the pharmaceutical composition comprises at least one pharmaceutically acceptable carrier and a compound of Formula (I), or a pharmaceutically acceptable salt thereof, wherein R₁ is hydrogen or acetyl; R₂, R₃, R₄, and R₅ are independently hydrogen, trifluoromethyl, or 2,4-difluorophenyl; and R₆ is (L) N-acetylcysteine.

In another aspect, the present invention provides uses for compounds of Formula (I) for preparing, or for the manufacture of, a medicament for treating atherosclerosis, neuropathy, nephropathy, retinopathy, inflammatory disorders, cardiovascular diseases, and metabolic disorders in a mammal or patient, wherein the compound of Formula (I) is selected from Example 1, 2, 3, 4, 5, 6, 7, 8, 9, 10, 11, 12, 13, 14, 15, 16, 17, 18, 19, 20, or 21.

In certain embodiments, the present invention provides uses for compounds of Formula (I) for preparing, or for the manufacture of, a medicament for treating dyslipidemia, insulin resistance, β-cell dysfunction, hyperglycemia, metabolic syndrome, and any form of diabetes mellitus including type I and type II diabetes, in a patient, wherein the compound of Formula (I) is selected from Example 1, 2, 3, 4, 5, 6, 7, 8, 9, 10, 11, 12, 13, 14, 15, 16, 17, 18, 19, 20, or 21.

In certain embodiments, the present invention provides uses for compounds of Formula (I) for preparing, or for the manufacture of, a medicament for reducing advanced glycated end products and/or lipid peroxidation including, but not limited to, oxidation of low-density lipoproteins in a patient, wherein the compound of Formula (I) is selected from Example 1, 2, 3, 4, 5, 6, 7, 8, 9, 10, 11, 12, 13, 14, 15, 16, 17, 18, 19, 20, or 21.

In certain embodiments, the present invention provides uses for pharmaceutical compositions for preparing, or for the manufacture of, a medicament for treating dyslipidemia, insulin resistance, β-cell dysfunction, hyperglycemia, metabolic syndrome, and any form of diabetes mellitus including type I and type II diabetes, in a patient, wherein the pharmaceutical composition comprises at least one pharmaceutically acceptable carrier and a compound of Formula (I), or a pharmaceutically acceptable salt thereof, wherein the compound of Formula (I) is selected from Example 1, 2, 3, 4, 5, 6, 7, 8, 9, 10, 11, 12, 13, 14, 15, 16, 17, 18, 19, 20, or 21.

In certain embodiments, the present invention provides uses for pharmaceutical compositions for preparing, or for the manufacture of, a medicament for reducing advanced glycated end products and/or lipid peroxidation including, but not limited to, oxidation of low-density lipoproteins in a patient, wherein the pharmaceutical composition comprises at least one pharmaceutically acceptable carrier and a compound of Formula (I), or a pharmaceutically acceptable salt thereof, wherein the compound of Formula (I) is selected from Example 1, 2, 3, 4, 5, 6, 7, 8, 9, 10, 11, 12, 13, 14, 15, 16, 17, 18, 19, 20, or 21.

In certain embodiments, the present invention provides uses for pharmaceutical compositions for preparing, or for the manufacture of, a medicament for reducing triglycerides and/or free fatty acids in a patient, wherein the pharmaceutical composition comprises at least one pharmaceutically acceptable carrier and a compound of Formula (I), or a pharmaceutically acceptable salt thereof, wherein the compound of Formula (I) is selected from Example 1, 2, 3, 4, 5, 6, 7, 8, 9, 10, 11, 12, 13, 14, 15, 16, 17, or 18.

In another aspect, the present invention provides uses for compounds of Formula (I) for preparing, or for the manufacture of, a medicament for treating atherosclerosis, neuropathy, nephropathy, retinopathy, inflammatory disorders, cardiovascular diseases, and metabolic disorders in a mammal or patient, wherein the compound of Formula (I) is Example 1 (salnacedin).

In certain embodiments, the present invention provides uses for compounds of Formula (I) for preparing, or for the manufacture of, a medicament for treating dyslipidemia, insulin resistance, β-cell dysfunction, hyperglycemia, metabolic syndrome, and any form of diabetes mellitus including type I and type II diabetes, in a patient, wherein the compound of Formula (I) is Example 1 (salnacedin).

In certain embodiments, the present invention provides uses for compounds of Formula (I) for preparing, or for the manufacture of, a medicament for reducing advanced glycated end products and/or lipid peroxidation including, but not limited to, oxidation of low-density lipoproteins in a patient, wherein the compound of Formula (I) is Example 1 (salnacedin).

In certain embodiments, the present invention provides uses for compounds of Formula (I) for preparing, or for the manufacture of, a medicament for reducing triglycerides and/or free fatty acids in a patient, wherein the compound of Formula (I) is Example 1 (salnacedin).

In certain embodiments, the present invention provides uses for pharmaceutical compositions for preparing, or for the manufacture of, a medicament for treating dyslipidemia, insulin resistance, β-cell dysfunction, hyperglycemia, metabolic syndrome, and any form of diabetes mellitus including type I and type II diabetes, in a patient, wherein the pharmaceutical composition comprises at least one pharmaceutically acceptable carrier and a compound of Formula (I), or a pharmaceutically acceptable salt thereof, wherein the compound of Formula (I) is Example 1 (salnacedin).

In certain embodiments, the present invention provides uses for pharmaceutical compositions for preparing, or for the manufacture of, a medicament for reducing advanced glycated end products and/or lipid peroxidation including, but not limited to, oxidation of low-density lipoproteins in a patient, wherein the pharmaceutical composition comprises at least one pharmaceutically acceptable carrier and a compound of Formula (I), or a pharmaceutically acceptable salt thereof, wherein the compound of Formula (I) is Example 1 (salnacedin).

In certain embodiments, the present invention provides uses for pharmaceutical compositions for preparing, or for the manufacture of, a medicament for reducing triglycerides and/or free fatty acids in a patient, wherein the pharmaceutical composition comprises at least one pharmaceutically acceptable carrier and a compound of Formula (I), or a pharmaceutically acceptable salt thereof, wherein the compound of Formula (I) is Example 1 (salnacedin).

In another aspect, the present invention provides uses for compounds of Formula (I) for preparing, or for the manufacture of, a medicament for treating atherosclerosis, neuropathy, nephropathy, retinopathy, inflammatory disorders, cardiovascular diseases, and metabolic disorders in a mammal or patient, wherein the compound of Formula (I) is Example 4.

In certain embodiments, the present invention provides uses for compounds of Formula (I) for preparing, or for the manufacture of, a medicament for treating dyslipidemia, insulin resistance, β-cell dysfunction, hyperglycemia, metabolic syndrome, and any form of diabetes mellitus including type I and type II diabetes, in a patient, wherein the compound of Formula (I) is Example 4.

In certain embodiments, the present invention provides uses for compounds of Formula (I) for preparing, or for the manufacture of, a medicament for reducing advanced glycated end products and/or lipid peroxidation including, but not limited to, oxidation of low-density lipoproteins in a patient, wherein the compound of Formula (I) is Example 4.

In certain embodiments, the present invention provides uses for pharmaceutical compositions for preparing, or for the manufacture of, a medicament for treating dyslipidemia, insulin resistance, β-cell dysfunction, hyperglycemia, metabolic syndrome, and any form of diabetes mellitus including type I and type II diabetes, in a patient, wherein the pharmaceutical composition comprises at least one pharmaceutically acceptable carrier and a compound of Formula (I), or a pharmaceutically acceptable salt thereof, wherein the compound of Formula (I) is Example 4.

In certain embodiments, the present invention provides uses for pharmaceutical compositions for preparing, or for the manufacture of, a medicament for reducing advanced glycated end products and/or lipid peroxidation including, but not limited to, oxidation of low-density lipoproteins in a patient, wherein the pharmaceutical composition comprises at least one pharmaceutically acceptable carrier and a compound of Formula (I), or a pharmaceutically acceptable salt thereof, wherein the compound of Formula (I) is Example 4.

In another aspect, the present invention provides the uses for compounds of Formula (I) for preparing, or for the manufacture of, a medicament for treating atherosclerosis, neuropathy, nephropathy, retinopathy, inflammatory disorders, cardiovascular diseases, and metabolic disorders in a mammal or patient, wherein the compound of Formula (I) is Example 7.

In certain embodiments, the present invention provides uses for compounds of Formula (I) for preparing, or for the manufacture of, a medicament for treating dyslipidemia, insulin resistance, β-cell dysfunction, hyperglycemia, metabolic syndrome, and any form of diabetes mellitus including type I and type II diabetes, in a patient, wherein the compound of Formula (I) is Example 7.

In certain embodiments, the present invention provides uses for compounds of Formula (I) for preparing, or for the manufacture of, a medicament for reducing advanced glycated end products and/or lipid peroxidation including, but not limited to, oxidation of low-density lipoproteins in a patient, wherein the compound of Formula (I) is Example 7.

In certain embodiments, the present invention provides uses for pharmaceutical compositions for preparing, or for the manufacture of, a medicament for treating dyslipidemia, insulin resistance, β-cell dysfunction, hyperglycemia, metabolic syndrome, and any form of diabetes mellitus including type I and type II diabetes, in a patient, wherein the pharmaceutical composition comprises at least one pharmaceutically acceptable carrier and a compound of Formula (I), or a pharmaceutically acceptable salt thereof, wherein the compound of Formula (I) is Example 7.

In certain embodiments, the present invention provides uses for pharmaceutical compositions for preparing, or for the manufacture of, a medicament for reducing advanced glycated end products and/or lipid peroxidation including, but not limited to, oxidation of low-density lipoproteins in a patient, wherein the pharmaceutical composition comprises at least one pharmaceutically acceptable carrier and a compound of Formula (I), or a pharmaceutically acceptable salt thereof, wherein the compound of Formula (I) is Example 7.

In another aspect, the present invention provides uses for compounds of Formula (I) for preparing, or for the manufacture of, a medicament for treating atherosclerosis, neuropathy, nephropathy, retinopathy, inflammatory disorders, cardiovascular diseases, and metabolic disorders in a mammal or patient, wherein the compound of Formula (I) is Example 10.

In certain embodiments, the present invention provides uses for compounds of Formula (I) for preparing, or for the manufacture of, a medicament for reducing advanced glycated end products and/or lipid peroxidation including, but not limited to, oxidation of low-density lipoproteins in a patient, wherein the compound of Formula (I) is Example 10.

In certain embodiments, the present invention provides uses for compounds of Formula (I) for preparing, or for the manufacture of, a medicament for treating dyslipidemia, insulin resistance, β-cell dysfunction, hyperglycemia, metabolic syndrome, and any form of diabetes mellitus including type I and type II diabetes, in a patient, wherein the compound of Formula (I) is Example 10.

In certain embodiments, the present invention provides uses for pharmaceutical compositions for preparing, or for the manufacture of, a medicament for treating dyslipidemia, insulin resistance, β-cell dysfunction, hyperglycemia, metabolic syndrome, and any form of diabetes mellitus including type I and type II diabetes, in a patient, wherein the pharmaceutical composition comprises at least one pharmaceutically acceptable carrier and a compound of Formula (I), or a pharmaceutically acceptable salt thereof, wherein the compound of Formula (I) is Example 10.

In certain embodiments, the present invention provides uses for pharmaceutical compositions for preparing, or for the manufacture of, a medicament for reducing advanced glycated end products and/or lipid peroxidation including, but not limited to, oxidation of low-density lipoproteins in a patient, wherein the pharmaceutical composition comprises at least one pharmaceutically acceptable carrier and a compound of Formula (I), or a pharmaceutically acceptable salt thereof, wherein the compound of Formula (I) is Example 10.

In another aspect, the present invention provides uses for compounds of Formula (I) for preparing, or for the manufacture of, a medicament for treating atherosclerosis, neuropathy, nephropathy, retinopathy, inflammatory disorders, cardiovascular diseases, and metabolic disorders in a mammal or patient, wherein the compound of Formula (I) is Example 13.

In certain embodiments, the present invention provides uses for compounds of Formula (I) for preparing, or for the manufacture of, a medicament for treating dyslipidemia, insulin resistance, β-cell dysfunction, hyperglycemia, metabolic syndrome, and any form of diabetes mellitus including type I and type II diabetes, in a patient, wherein the compound of Formula (I) is Example 13.

In certain embodiments, the present invention provides uses for compounds of Formula (I) for preparing, or for the manufacture of, a medicament for reducing advanced glycated end products and/or lipid peroxidation including, but not limited to, oxidation of low-density lipoproteins in a patient, wherein the compound of Formula (I) is Example 13.

In certain embodiments, the present invention provides uses for compounds of Formula (I) for preparing, or for the manufacture of, a medicament for reducing triglycerides and/or free fatty acids in a patient, wherein the compound of Formula (I) is Example 13.

In certain embodiments, the present invention provides uses for pharmaceutical compositions for preparing, or for the manufacture of, a medicament for treating dyslipidemia, insulin resistance, β-cell dysfunction, hyperglycemia, metabolic syndrome, and any form of diabetes mellitus including type I and type II diabetes, in a patient, wherein the pharmaceutical composition comprises at least one pharmaceutically acceptable carrier and a compound of Formula (I), or a pharmaceutically acceptable salt thereof, wherein the compound of Formula (I) is Example 13.

In certain embodiments, the present invention provides uses for pharmaceutical compositions for preparing, or for the manufacture of, a medicament for reducing advanced glycated end products and/or lipid peroxidation including, but not limited to, oxidation of low-density lipoproteins in a patient, wherein the pharmaceutical composition comprises at least one pharmaceutically acceptable carrier and a compound of Formula (I), or a pharmaceutically acceptable salt thereof, wherein the compound of Formula (I) is Example 13.

In certain embodiments, the present invention provides uses for pharmaceutical compositions for preparing, or for the manufacture of, a medicament for reducing triglycerides and/or free fatty acids in a patient, wherein the pharmaceutical composition comprises at least one pharmaceutically acceptable carrier and a compound of Formula (I), or a pharmaceutically acceptable salt thereof, wherein the compound of Formula (I) is Example 13.

In another aspect, the present invention provides uses for compounds of Formula (I) for preparing, or for the manufacture of, a medicament for treating atherosclerosis, neuropathy, nephropathy, retinopathy, inflammatory disorders, cardiovascular diseases, and metabolic disorders in a mammal or patient, wherein the compound of Formula (I) is Example 16.

In certain embodiments, the present invention provides uses for compounds of Formula (I) for preparing, or for the manufacture of, a medicament for treating dyslipidemia, insulin resistance, β-cell dysfunction, hyperglycemia, metabolic syndrome, and any form of diabetes mellitus including type I and type II diabetes, in a patient, wherein the compound of Formula (I) is Example 16.

In certain embodiments, the present invention provides uses for compounds of Formula (I) for preparing, or for the manufacture of, a medicament for reducing advanced glycated end products and/or lipid peroxidation including, but not limited to, oxidation of low-density lipoproteins in a patient, wherein the compound of Formula (I) is Example 16.

In certain embodiments, the present invention provides uses for pharmaceutical compositions for preparing, or for the manufacture of, a medicament for treating dyslipidemia, insulin resistance, β-cell dysfunction, hyperglycemia, metabolic syndrome, and any form of diabetes mellitus including type I and type II diabetes, in a patient, wherein the pharmaceutical composition comprises at least one pharmaceutically acceptable carrier and a compound of Formula (I), or a pharmaceutically acceptable salt thereof, wherein the compound of Formula (I) is Example 16.

In certain embodiments, the present invention provides uses for pharmaceutical compositions for preparing, or for the manufacture of, a medicament for reducing advanced glycated end products and/or lipid peroxidation including, but not limited to, oxidation of low-density lipoproteins in a patient, wherein the pharmaceutical composition comprises at least one pharmaceutically acceptable carrier and a compound of Formula (I), or a pharmaceutically acceptable salt thereof, wherein the compound of Formula (I) is Example 16.

In another aspect, the present invention provides uses for compounds of Formula (I) for preparing, or for the manufacture of, a medicament for treating atherosclerosis, neuropathy, nephropathy, retinopathy, inflammatory disorders, cardiovascular diseases, and metabolic disorders in a mammal or patient, wherein the compound of Formula (I) is selected from Example 19, 20, or 21.

In certain embodiments, the present invention provides uses for compounds of Formula (I) for preparing, or for the manufacture of, a medicament for treating dyslipidemia, insulin resistance, β-cell dysfunction, hyperglycemia, metabolic syndrome, and any form of diabetes mellitus including type I and type II diabetes, in a patient, wherein the compound of Formula (I) is selected from Example 19, 20, or 21.

In certain embodiments, the present invention provides uses for compounds of Formula (I) for preparing, or for the manufacture of, a medicament for reducing advanced glycated end products and/or lipid peroxidation including, but not limited to, oxidation of low-density lipoproteins in a patient, wherein the compound of Formula (I) is selected from Example 19, 20, or 21.

In certain embodiments, the present invention provides uses for compounds of Formula (I) for preparing, or for the manufacture of, a medicament for reducing triglycerides and/or free fatty acids in a patient, wherein the compound of Formula (I) is selected from Example 19, 20, or 21.

In certain embodiments, the present invention provides uses for pharmaceutical compositions for preparing, or for the manufacture of, a medicament for treating dyslipidemia, insulin resistance, β-cell dysfunction, hyperglycemia, metabolic syndrome, and any form of diabetes mellitus including type I and type II diabetes, in a patient, wherein the pharmaceutical composition comprises at least one pharmaceutically acceptable carrier and a compound of Formula (I), or a pharmaceutically acceptable salt thereof, wherein the compound of Formula (I) is selected from Example 19, 20, or 21.

In certain embodiments, the present invention provides uses for pharmaceutical compositions for preparing, or for the manufacture of, a medicament for reducing advanced glycated end products and/or lipid peroxidation including, but not limited to, oxidation of low-density lipoproteins in a patient, wherein the pharmaceutical composition comprises at least one pharmaceutically acceptable carrier and a compound of Formula (I), or a pharmaceutically acceptable salt thereof, wherein the compound of Formula (I) is selected from Example 19, 20, or 21.

In certain embodiments, the present invention provides uses for pharmaceutical compositions for preparing, or for the manufacture of, a medicament for reducing triglycerides and/or free fatty acids in a patient, wherein the pharmaceutical composition comprises at least one pharmaceutically acceptable carrier and a compound of Formula (I), or a pharmaceutically acceptable salt thereof, wherein the compound of Formula (I) is selected from Example 19, 20, or 21.

In another aspect, the present invention provides uses for compounds of Formula (I) for preparing, or for the manufacture of, a medicament for treating atherosclerosis, neuropathy, nephropathy, retinopathy, inflammatory disorders, cardiovascular diseases, and metabolic disorders in a mammal or patient, wherein R₁ is hydrogen or acetyl; R₂, R₃, R₄, and R₅ are independently hydrogen, halo(C₁-C₆)alkyl, or halogen; R₆ is —NZ₅Z₆; Z₅ is hydrogen; Z₆ is hydrogen, (C₁-C₆)alkyl, (C₁-C₆)alkylcarbonyl, phenyl, phenyl(CH₂)—, or phenyl(CH₂)₂—, wherein the phenyl is optionally substituted with 1, 2, 3, 4, or 5 groups that are independently (C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ₇Z₈, or (NZ₇Z₈)carbonyl; and Z₇ and Z₈ are independently hydrogen, (C₁-C₆)alkyl, or (C₁-C₆)alkylcarbonyl.

In certain embodiments, the present invention provides uses for compounds of Formula (I) for preparing, or for the manufacture of, a medicament for treating dyslipidemia, insulin resistance, β-cell dysfunction, hyperglycemia, metabolic syndrome, and any form of diabetes mellitus including type I and type II diabetes, in a patient, wherein R₁ is hydrogen or acetyl; R₂, R₃, R₄, and R₅ are independently hydrogen, halo(C₁-C₆)alkyl, or halogen; R₆ is —NZ₅Z₆; Z₅ is hydrogen; Z₆ is hydrogen, (C₁-C₆)alkyl, (C₁-C₆)alkylcarbonyl, phenyl, phenyl(CH₂)—, or phenyl(CH₂)₂—, wherein the phenyl is optionally substituted with 1, 2, 3, 4, or 5 groups that are independently (C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ₇Z₈, or (NZ₇Z₈)carbonyl; and Z₇ and Z₈ are independently hydrogen, (C₁-C₆)alkyl, or (C₁-C₆)alkylcarbonyl.

In certain embodiments, the present invention provides uses for compounds of Formula (I) for preparing, or for the manufacture of, a medicament for reducing advanced glycated end products and/or lipid peroxidation including, but not limited to, oxidation of low-density lipoproteins in a patient, wherein R₁ is hydrogen or acetyl; R₂, R₃, R₄, and R₅ are independently hydrogen, halo(C₁-C₆)alkyl, or halogen; R₆ is —NZ₅Z₆; Z₅ is hydrogen; Z₆ is hydrogen, (C₁-C₆)alkyl, (C₁-C₆)alkylcarbonyl, phenyl, phenyl(CH₂)—, or phenyl(CH₂)₂₋₅ wherein the phenyl is optionally substituted with 1, 2, 3, 4, or 5 groups that are independently (C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ₇Z₈, or (NZ₇Z₈)carbonyl; and Z₇ and Z₈ are independently hydrogen, (C₁-C₆)alkyl, or (C₁-C₆)alkylcarbonyl.

In certain embodiments, the present invention provides uses for pharmaceutical compositions for preparing, or for the manufacture of, a medicament for treating dyslipidemia, insulin resistance, β-cell dysfunction, hyperglycemia, metabolic syndrome, and any form of diabetes mellitus including type I and type II diabetes, in a patient, wherein the pharmaceutical composition comprises at least one pharmaceutically acceptable carrier and a compound of Formula (I), or a pharmaceutically acceptable salt thereof, wherein R₁ is hydrogen or acetyl; R₂, R₃, R₄, and R₅ are independently hydrogen, halo(C₁-C₆)alkyl, or halogen; R₆ is —NZ₅Z₆; Z₅ is hydrogen; Z₆ is hydrogen, (C₁-C₆)alkyl, (C₁-C₆)alkylcarbonyl, phenyl, phenyl(CH₂)—, or phenyl(CH₂)₂—, wherein the phenyl is optionally substituted with 1, 2, 3, 4, or 5 groups that are independently (C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ₇Z₈, or (NZ₇Z₈)carbonyl; and Z₇ and Z₈ are independently hydrogen, (C₁-C₆)alkyl, or (C₁-C₆)alkylcarbonyl.

In certain embodiments, the present invention provides uses for pharmaceutical compositions for preparing, or for the manufacture of, a medicament for reducing advanced glycated end products and/or lipid peroxidation including, but not limited to, oxidation of low-density lipoproteins in a patient, wherein the pharmaceutical composition comprises at least one pharmaceutically acceptable carrier and a compound of Formula (I), or a pharmaceutically acceptable salt thereof, wherein R₁ is hydrogen or acetyl; R₂, R₃, R₄, and R₅ are independently hydrogen, halo(C₁-C₆)alkyl, or halogen; R₆ is —NZ₅Z₆; Z₅ is hydrogen; Z₆ is hydrogen, (C₁-C₆)alkyl, (C₁-C₆)alkylcarbonyl, phenyl, phenyl(CH₂)—, or phenyl(CH₂)₂—, wherein the phenyl is optionally substituted with 1, 2, 3, 4, or 5 groups that are independently (C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ₇Z₈, or (NZ₇Z₈)carbonyl; and Z₇ and Z₈ are independently hydrogen, (C₁-C₆)alkyl, or (C₁-C₆)alkylcarbonyl.

In another aspect, the present invention provides uses for compounds of Formula (I) for preparing, or for the manufacture of, a medicament for treating atherosclerosis, neuropathy, nephropathy, retinopathy, inflammatory disorders, cardiovascular diseases, and metabolic disorders in a mammal or patient, wherein R₁ is hydrogen or acetyl; R₂, R₃, R₄, and R₅ are independently hydrogen, halo(C₁-C₆)alkyl, or halogen; R₆ is —NZ₅Z₆; Z₅ is hydrogen; Z₆ is hydrogen, (C₁-C₆)alkyl, (C₁-C₆)alkylcarbonyl. In certain embodiments, the present invention provides uses for compounds of Formula (I) for preparing, or for the manufacture of, a medicament for treating dyslipidemia, insulin resistance, β-cell dysfunction, hyperglycemia, metabolic syndrome, and any form of diabetes mellitus including type I and type II diabetes, in a patient, wherein R₁ is hydrogen or acetyl; R₂, R₃, R₄, and R₅ are independently hydrogen, halo(C₁-C₆)alkyl, or halogen; R₆ is —NZ₅Z₆; Z₅ is hydrogen; Z₆ is hydrogen, (C₁-C₆)alkyl, (C₁-C₆)alkylcarbonyl.

In certain embodiments, the present invention provides uses for compounds of Formula (I) for preparing, or for the manufacture of, a medicament for reducing advanced glycated end products and/or lipid peroxidation including, but not limited to, oxidation of low-density lipoproteins in a patient, wherein R₁ is hydrogen or acetyl; R₂, R₃, R₄, and R₅ are independently hydrogen, halo(C₁-C₆)alkyl, or halogen; R₆ is —NZ₅Z₆; Z₅ is hydrogen; Z₆ is hydrogen, (C₁-C₆)alkyl, (C₁-C₆)alkylcarbonyl.

In certain embodiments, the present invention provides uses for pharmaceutical compositions for preparing, or for the manufacture of, a medicament for treating dyslipidemia, insulin resistance, β-cell dysfunction, hyperglycemia, metabolic syndrome, and any form of diabetes mellitus including type I and type II diabetes, in a patient, wherein the pharmaceutical composition comprises at least one pharmaceutically acceptable carrier and a compound of Formula (I), or a pharmaceutically acceptable salt thereof, wherein R₁ is hydrogen or acetyl; R₂, R₃, R₄, and R₅ are independently hydrogen, halo(C₁-C₆)alkyl, or halogen; R₆ is —NZ₅Z₆; Z₅ is hydrogen; Z₆ is hydrogen, (C₁-C₆)alkyl, (C₁-C₆)alkylcarbonyl.

In certain embodiments, the present invention provides uses for pharmaceutical compositions for preparing, or for the manufacture of, a medicament for reducing advanced glycated end products and/or lipid peroxidation including, but not limited to, oxidation of low-density lipoproteins in a patient, wherein the pharmaceutical composition comprises at least one pharmaceutically acceptable carrier and a compound of Formula (I), or a pharmaceutically acceptable salt thereof, wherein R₁ is hydrogen or acetyl; R₂, R₃, R₄, and R₅ are independently hydrogen, halo(C₁-C₆)alkyl, or halogen; R₆ is —NZ₅Z₆; Z₅ is hydrogen; Z₆ is hydrogen, (C₁-C₆)alkyl, (C₁-C₆)alkylcarbonyl.

In another aspect, the present invention provides uses for compounds of Formula (I) for preparing, or for the manufacture of, a medicament for treating atherosclerosis, neuropathy, nephropathy, retinopathy, inflammatory disorders, cardiovascular diseases, and metabolic disorders in a mammal or patient, wherein R₁ is hydrogen or acetyl; R₂, R₃, R₄, and R₅ are independently hydrogen, trifluoromethyl, or Cl; R₆ is —NZ₅Z₆; Z₅ is hydrogen; and Z₆ is hydrogen.

In certain embodiments, the present invention provides uses for compounds of Formula (I) for preparing, or for the manufacture of, a medicament for treating dyslipidemia, insulin resistance, β-cell dysfunction, hyperglycemia, metabolic syndrome, and any form of diabetes mellitus including type I and type II diabetes, in a patient, wherein R₁ is hydrogen or acetyl; R₂, R₃, R₄, and R₅ are independently hydrogen, trifluoromethyl, or Cl; R₆ is —NZ₅Z₆; Z₅ is hydrogen; and Z₆ is hydrogen.

In certain embodiments, the present invention provides uses for compounds of Formula (I) for preparing, or for the manufacture of, a medicament for reducing advanced glycated end products and/or lipid peroxidation including, but not limited to, oxidation of low-density lipoproteins in a patient, wherein R₁ is hydrogen or acetyl; R₂, R₃, R₄, and R₅ are independently hydrogen, trifluoromethyl, or Cl; R₆ is —NZ₅Z₆; Z₅ is hydrogen; and Z₆ is hydrogen.

In certain embodiments, the present invention provides uses for pharmaceutical compositions for preparing, or for the manufacture of, a medicament for treating dyslipidemia, insulin resistance, β-cell dysfunction, hyperglycemia, metabolic syndrome, and any form of diabetes mellitus including type I and type II diabetes, in a patient, wherein the pharmaceutical composition comprises at least one pharmaceutically acceptable carrier and a compound of Formula (I), or a pharmaceutically acceptable salt thereof, wherein R₁ is hydrogen or acetyl; R₂, R₃, R₄, and R₅ are independently hydrogen, trifluoromethyl, or Cl; R₆ is —NZ₅Z₆; Z₅ is hydrogen; and Z₆ is hydrogen.

In certain embodiments, the present invention provides uses for pharmaceutical compositions for preparing, or for the manufacture of, a medicament for reducing advanced glycated end products and/or lipid peroxidation including, but not limited to, oxidation of low-density lipoproteins in a patient, wherein the pharmaceutical composition comprises at least one pharmaceutically acceptable carrier and a compound of Formula (I), or a pharmaceutically acceptable salt thereof, wherein R₁ is hydrogen or acetyl; R₂, R₃, R₄, and R₅ are independently hydrogen, trifluoromethyl, or Cl; R₆ is —NZ₅Z₆; Z₅ is hydrogen; and Z₆ is hydrogen.

In another aspect, the present invention provides uses for compounds of Formula (I) for preparing, or for the manufacture of, a medicament for treating atherosclerosis, neuropathy, nephropathy, retinopathy, inflammatory disorders, cardiovascular diseases, and metabolic disorders in a mammal or patient, wherein R₁ is hydrogen or acetyl; R₂, R₃, R₄, and R₅ are independently hydrogen, halo(C₁-C₆)alkyl, or halogen; R₆ is —NZ₅Z₆; Z₅ is hydrogen; Z₆ is phenyl, wherein the phenyl is optionally substituted with 1, 2, 3, 4, or 5 groups that are independently (C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ₇Z₈, or (NZ₇Z₈)carbonyl; and Z₇ and Z₈ are independently hydrogen, (C₁-C₆)alkyl, or (C₁-C₆)alkylcarbonyl.

In certain embodiments, the present invention provides uses for compounds of Formula (I) for preparing, or for the manufacture of, a medicament for treating dyslipidemia, insulin resistance, β-cell dysfunction, hyperglycemia, metabolic syndrome, and any form of diabetes mellitus including type I and type II diabetes, in a patient, wherein R₁ is hydrogen or acetyl; R₂, R₃, R₄, and R₅ are independently hydrogen, halo(C₁-C₆)alkyl, or halogen; R₆ is —NZ₅Z₆; Z₅ is hydrogen; Z₆ is phenyl, wherein the phenyl is optionally substituted with 1, 2, 3, 4, or 5 groups that are independently (C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ₇Z₈, or (NZ₇Z₈)carbonyl; and Z₇ and Z₈ are independently hydrogen, (C₁-C₆)alkyl, or (C₁-C₆)alkylcarbonyl.

In certain embodiments, the present invention provides uses for compounds of Formula (I) for preparing, or for the manufacture of, a medicament for reducing advanced glycated end products and/or lipid peroxidation including, but not limited to, oxidation of low-density lipoproteins in a patient, wherein R₁ is hydrogen or acetyl; R₂, R₃, R₄, and R₅ are independently hydrogen, halo(C₁-C₆)alkyl, or halogen; R₆ is —NZ₅Z₆; Z₅ is hydrogen; Z₆ is phenyl, wherein the phenyl is optionally substituted with 1, 2, 3, 4, or 5 groups that are independently (C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ₇Z₈, or (NZ₇Z₈)carbonyl; and Z₇ and Z₈ are independently hydrogen, (C₁-C₆)alkyl, or (C₁-C₆)alkylcarbonyl.

In certain embodiments, the present invention provides uses for pharmaceutical compositions for preparing, or for the manufacture of, a medicament for treating dyslipidemia, insulin resistance, β-cell dysfunction, hyperglycemia, metabolic syndrome, and any form of diabetes mellitus including type I and type II diabetes, in a patient, wherein the pharmaceutical composition comprises at least one pharmaceutically acceptable carrier and a compound of Formula (I), or a pharmaceutically acceptable salt thereof, wherein R₁ is hydrogen or acetyl; R₂, R₃, R₄, and R₅ are independently hydrogen, halo(C₁-C₆)alkyl, or halogen; R₆ is —NZ₅Z₆; Z₅ is hydrogen; Z₆ is phenyl, wherein the phenyl is optionally substituted with 1, 2, 3, 4, or 5 groups that are independently (C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ₇Z₈, or (NZ₇Z₈)carbonyl; and Z₇ and Z₈ are independently hydrogen, (C₁-C₆)alkyl, or (C₁-C₆)alkylcarbonyl.

In certain embodiments, the present invention provides uses for pharmaceutical compositions for preparing, or for the manufacture of, a medicament for reducing advanced glycated end products and/or lipid peroxidation including, but not limited to, oxidation of low-density lipoproteins in a patient, wherein the pharmaceutical composition comprises at least one pharmaceutically acceptable carrier and a compound of Formula (I), or a pharmaceutically acceptable salt thereof, wherein R₁ is hydrogen or acetyl; R₂, R₃, R₄, and R₅ are independently hydrogen, halo(C₁-C₆)alkyl, or halogen; R₆ is —NZ₅Z₆; Z₅ is hydrogen; Z₆ is phenyl, wherein the phenyl is optionally substituted with 1, 2, 3, 4, or 5 groups that are independently (C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ₇Z₈, or (NZ₇Z₈)carbonyl; and Z₇ and Z₈ are independently hydrogen, (C₁-C₆)alkyl, or (C₁-C₆)alkylcarbonyl.

In another aspect, the present invention provides uses for compounds of Formula (I) for preparing, or for the manufacture of, a medicament for treating atherosclerosis, neuropathy, nephropathy, retinopathy, inflammatory disorders, cardiovascular diseases, and metabolic disorders in a mammal or patient, wherein R₁ is hydrogen or acetyl; R₂, R₃, R₄, and R₅ are independently hydrogen, halo(C₁-C₆)alkyl, or halogen; R₆ is —NZ₅Z₆; Z₅ is hydrogen; Z₆ is phenyl, wherein the phenyl is optionally substituted with 1 or 2 groups that are independently halo(C₁-C₆)alkyl or halogen.

In certain embodiments, the present invention provides uses for compounds of Formula (I) for preparing, or for the manufacture of, a medicament for treating dyslipidemia, insulin resistance, β-cell dysfunction, hyperglycemia, metabolic syndrome, and any form of diabetes mellitus including type I and type II diabetes, in a patient, wherein R₁ is hydrogen or acetyl; R₂, R₃, R₄, and R₅ are independently hydrogen, halo(C₁-C₆)alkyl, or halogen; R₆ is —NZ₅Z₆; Z₅ is hydrogen; Z₆ is phenyl, wherein the phenyl is optionally substituted with 1 or 2 groups that are independently halo(C₁-C₆)alkyl or halogen.

In certain embodiments, the present invention provides uses for compounds of Formula (I) for preparing, or for the manufacture of, a medicament for reducing advanced glycated end products and/or lipid peroxidation including, but not limited to, oxidation of low-density lipoproteins in a patient, wherein R₁ is hydrogen or acetyl; R₂, R₃, R₄, and R₅ are independently hydrogen, halo(C₁-C₆)alkyl, or halogen; R₆ is —NZ₅Z₆; Z₅ is hydrogen; Z₆ is phenyl, wherein the phenyl is optionally substituted with 1 or 2 groups that are independently halo(C₁-C₆)alkyl or halogen.

In certain embodiments, the present invention provides uses for pharmaceutical compositions for preparing, or for the manufacture of, a medicament for treating dyslipidemia, insulin resistance, β-cell dysfunction, hyperglycemia, metabolic syndrome, and any form of diabetes mellitus including type I and type II diabetes, in a patient, wherein the pharmaceutical composition comprises at least one pharmaceutically acceptable carrier and a compound of Formula (I), or a pharmaceutically acceptable salt thereof, wherein R₁ is hydrogen or acetyl; R₂, R₃, R₄, and R₅ are independently hydrogen, halo(C₁-C₆)alkyl, or halogen; R₆ is —NZ₅Z₆; Z₅ is hydrogen; Z₆ is phenyl, wherein the phenyl is optionally substituted with 1 or 2 groups that are independently halo(C₁-C₆)alkyl or halogen.

In certain embodiments, the present invention provides uses for pharmaceutical compositions for preparing, or for the manufacture of, a medicament for reducing advanced glycated end products and/or lipid peroxidation including, but not limited to, oxidation of low-density lipoproteins in a patient, wherein the pharmaceutical composition comprises at least one pharmaceutically acceptable carrier and a compound of Formula (I), or a pharmaceutically acceptable salt thereof, wherein R₁ is hydrogen or acetyl; R₂, R₃, R₄, and R₅ are independently hydrogen, halo(C₁-C₆)alkyl, or halogen; R₆ is —NZ₅Z₆; Z₅ is hydrogen; Z₆ is phenyl, wherein the phenyl is optionally substituted with 1 or 2 groups that are independently halo(C₁-C₆)alkyl or halogen.

In another aspect, the present invention provides uses for compounds of Formula (I) for preparing, or for the manufacture of, a medicament for treating atherosclerosis, neuropathy, nephropathy, retinopathy, inflammatory disorders, cardiovascular diseases, and metabolic disorders in a mammal or patient, wherein R₁ is hydrogen or acetyl; R₂, R₃, R₄, and R₅ are independently hydrogen, trifluoromethyl, or Cl; R₆ is —NZ₅Z₆; Z₅ is hydrogen; Z₆ is phenyl, wherein the phenyl is optionally substituted with 1 or 2 groups that are independently trifluoromethyll or Cl.

In certain embodiments, the present invention provides uses for compounds of Formula (I) for preparing, or for the manufacture of, a medicament for treating dyslipidemia, insulin resistance, β-cell dysfunction, hyperglycemia, metabolic syndrome, and any form of diabetes mellitus including type I and type II diabetes, in a patient, wherein R₁ is hydrogen or acetyl; R₂, R₃, R₄, and R₅ are independently hydrogen, trifluoromethyl, or Cl; R₆ is —NZ₅Z₆; Z₅ is hydrogen; Z₆ is phenyl, wherein the phenyl is optionally substituted with 1 or 2 groups that are independently trifluoromethyll or Cl.

In certain embodiments, the present invention provides uses for compounds of Formula (I) for preparing, or for the manufacture of, a medicament for reducing advanced glycated end products and/or lipid peroxidation including, but not limited to, oxidation of low-density lipoproteins in a patient, wherein R₁ is hydrogen or acetyl; R₂, R₃, R₄, and R₅ are independently hydrogen, trifluoromethyl, or Cl; R₆ is —NZ₅Z₆; Z₅ is hydrogen; Z₆ is phenyl, wherein the phenyl is optionally substituted with 1 or 2 groups that are independently trifluoromethyll or Cl.

In certain embodiments, the present invention provides uses for pharmaceutical compositions for preparing, or for the manufacture of, a medicament for treating dyslipidemia, insulin resistance, β-cell dysfunction, hyperglycemia, metabolic syndrome, and any form of diabetes mellitus including type I and type II diabetes, in a patient, wherein the pharmaceutical composition comprises at least one pharmaceutically acceptable carrier and a compound of Formula (I), or a pharmaceutically acceptable salt thereof, wherein R₁ is hydrogen or acetyl; R₂, R₃, R₄, and R₅ are independently hydrogen, trifluoromethyl, or Cl; R₆ is —NZ₅Z₆; Z₅ is hydrogen; Z₆ is phenyl, wherein the phenyl is optionally substituted with 1 or 2 groups that are independently trifluoromethyll or Cl.

In certain embodiments, the present invention provides uses for pharmaceutical compositions for preparing, or for the manufacture of, a medicament for reducing advanced glycated end products and/or lipid peroxidation including, but not limited to, oxidation of low-density lipoproteins in a patient, wherein the pharmaceutical composition comprises at least one pharmaceutically acceptable carrier and a compound of Formula (I), or a pharmaceutically acceptable salt thereof, wherein R₁ is hydrogen or acetyl; R₂, R₃, R₄, and R₅ are independently hydrogen, trifluoromethyl, or Cl; R₆ is —NZ₅Z₆; Z₅ is hydrogen; Z₆ is phenyl, wherein the phenyl is optionally substituted with 1 or 2 groups that are independently trifluoromethyll or Cl.

In another aspect, the present invention provides uses for compounds of Formula (I) or Formula (IV) for preparing, or for the manufacture of, a medicament for treating atherosclerosis, neuropathy, nephropathy, retinopathy, inflammatory disorders, cardiovascular diseases, and metabolic disorders in a mammal or patient, wherein the compound of Formula (I) or Formula (IV) is N-(3,5-bis(trifluoromethyl)phenyl)-5-chloro-2-hydroxybenzamide or 2-(3,5-bis(trifluoromethyl)phenylcarbamoyl)-4-chlorophenyl acetate.

In certain embodiments, the present invention provides uses for compounds of Formula (I) or Formula (IV) for preparing, or for the manufacture of, a medicament for treating dyslipidemia, insulin resistance, β-cell dysfunction, hyperglycemia, metabolic syndrome, and any form of diabetes mellitus including type I and type II diabetes, in a patient, wherein the compound of Formula (I) or Formula (IV) is N-(3,5-bis(trifluoromethyl)phenyl)-5-chloro-2-hydroxybenzamide or 2-(3,5-bis(trifluoromethyl)phenylcarbamoyl)-4-chlorophenyl acetate.

In certain embodiments, the present invention provides uses for compounds of Formula (I) or Formula (IV) for preparing, or for the manufacture of, a medicament for reducing advanced glycated end products and/or lipid peroxidation including, but not limited to, oxidation of low-density lipoproteins in a patient, wherein the compound of Formula (I) or Formula (IV) is N-(3,5-bis(trifluoromethyl)phenyl)-5-chloro-2-hydroxybenzamide or 2-(3,5-bis(trifluoromethyl)phenylcarbamoyl)-4-chlorophenyl acetate.

In certain embodiments, the present invention provides uses for pharmaceutical compositions for preparing, or for the manufacture of, a medicament for treating dyslipidemia, insulin resistance, β-cell dysfunction, hyperglycemia, metabolic syndrome, and any form of diabetes mellitus including type I and type II diabetes, in a patient, wherein the pharmaceutical composition comprises at least one pharmaceutically acceptable carrier and a compound of Formula (I) or Formula (IV), or a pharmaceutically acceptable salt thereof, wherein the compound of Formula (I) or Formula (IV) is N-(3,5-bis(trifluoromethyl)phenyl)-5-chloro-2-hydroxybenzamide or 2-(3,5-bis(trifluoromethyl)phenylcarbamoyl)-4-chlorophenyl acetate.

In certain embodiments, the present invention provides uses for pharmaceutical compositions for preparing, or for the manufacture of, a medicament for reducing advanced glycated end products and/or lipid peroxidation including, but not limited to, oxidation of low-density lipoproteins in a patient, wherein the pharmaceutical composition comprises at least one pharmaceutically acceptable carrier and a compound of Formula (I) or Formula (IV), or a pharmaceutically acceptable salt thereof, wherein the compound of Formula (I) or Formula (IV) is N-(3,5-bis(trifluoromethyl)phenyl)-5-chloro-2-hydroxybenzamide or 2-(3,5-bis(trifluoromethyl)phenylcarbamoyl)-4-chlorophenyl acetate.

In another aspect, the present invention provides compounds of Formula (I)

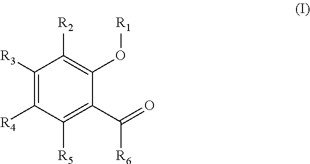

or a pharmaceutically acceptable salt thereof, wherein

R₁ is hydrogen, (C₁-C₆)alkylcarbonyl, or A;

R₂, R₃, R₄, and R₅ are independently hydrogen, (C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ₁Z₂, or (NZ₁Z₂)carbonyl, wherein the phenyl is optionally substituted with 1, 2, 3, 4, or 5 groups that are independently C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ₃Z₄, (NZ₃Z₄)carbonyl;

Z₁, Z₂, Z₃, and Z₄ are independently hydrogen, (C₁-C₆)alkyl, or (C₁-C₆)alkylcarbonyl;

R₆ is —NZ₅Z₆,

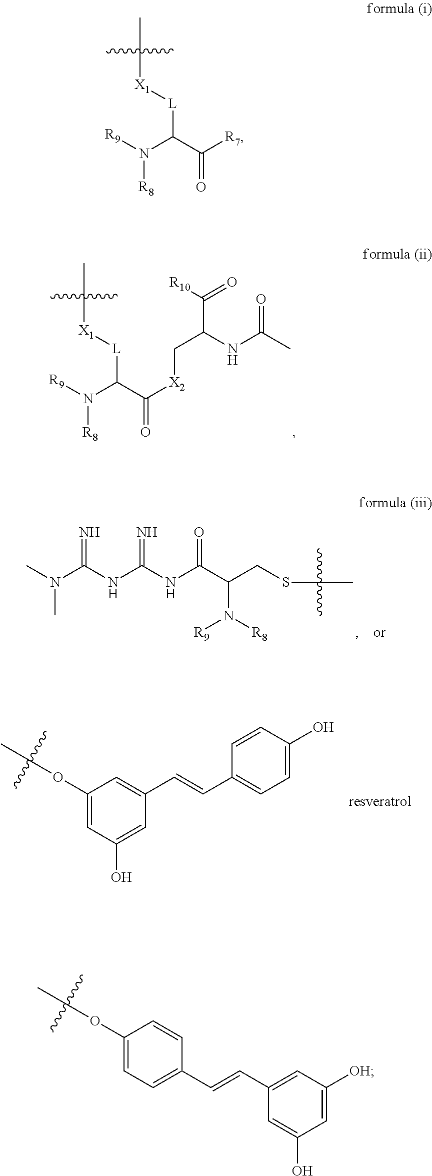

Z₅ and Z₆ are independently hydrogen, (C₁-C₆)alkyl, (C₁-C₆)alkylcarbonyl, phenyl, phenyl(CH₂)—, or phenyl(CH₂)₂—, wherein the phenyl is optionally substituted with 1, 2, 3, 4, or 5 groups that are independently (C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ₇Z₈, or (NZ₇Z₈)carbonyl;

Z₇ and Z₈ are independently hydrogen, (C₁-C₆)alkyl, or (C₁-C₆)alkylcarbonyl;

R₇ is (C₁-C₆)alkoxy, (C₁-C₆)alkyl, (C₁-C₆)alkylthio, hydroxy, or —NZ₉Z₁₀;

R₈ is hydrogen or (C₁-C₆)alkyl;

R₉ is hydrogen, (C₁-C₆)alkyl, or (C₁-C₆)alkylcarbonyl;

R₁₀ is (C₁-C₆)alkoxy, (C₁-C₆)alkyl, (C₁-C₆)alkylthio, hydroxy, or —NZ₉Z₁₀;

Z₉ and Z₁₀ are independently hydrogen, (C₁-C₆)alkyl, or (C₁-C₆)alkylcarbonyl;

A is

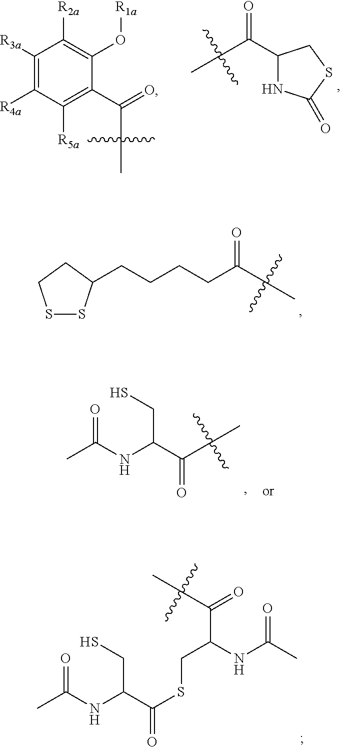

R₁ₐ is hydrogen, (C₁-C₆)alkylcarbonyl, or B;

R₂ₐ, R₃ₐ, R₄ₐ, and R₅ₐ are independently hydrogen, (C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ₁ₐZ₂ₐ, or (NZ₁ₐZ₂ₐ)carbonyl, wherein the phenyl is optionally substituted with 1, 2, 3, 4, or 5 groups that are independently C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ₃ₐZ₄ₐ, or (NZ₃ₐZ₄ₐ)carbonyl;
Z₁ₐ, Z₂ₐ, Z₃ₐ, and Z₄ₐ are independently hydrogen, (C₁-C₆)alkyl, or (C₁-C₆)alkylcarbonyl;

B is

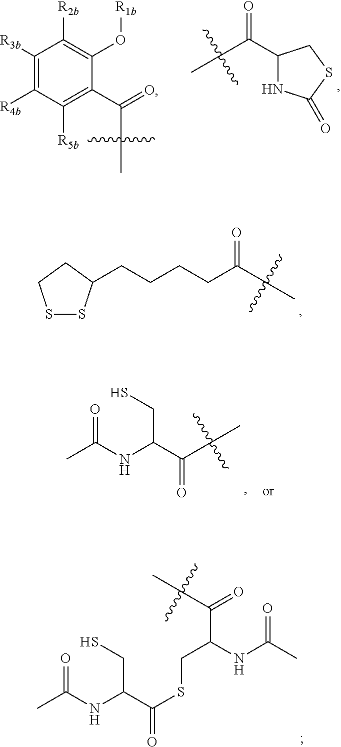

R_{1b} is hydrogen, (C₁-C₆)alkylcarbonyl, or C;

R_{2b}, R_{3b}, R_{4b}, and R_{5b} are independently hydrogen, (C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ_{1b}Z_{2b}, or (NZ_{1b}Z_{2b})carbonyl, wherein the phenyl is optionally substituted with 1, 2, 3, 4, or 5 groups that are independently C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ_{3b}Z_{4b}, or (NZ_{3b}Z_{4b})carbonyl;

Z_{1b}, Z_{2b}, Z_{3b}, and Z_{4b} are independently hydrogen, (C₁-C₆)alkyl, or (C₁-C₆)alkylcarbonyl; and

C is

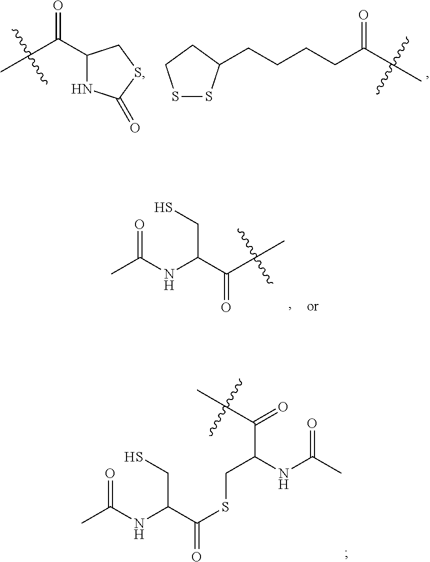

provided that Formula (I) does not encompass R₁ is hydrogen or acetyl; R₂, R₃, and R₅ are hydrogen; R₄ is H or 2,4-difluorophenyl; and R₆ is (L) N-acetylcysteine, (D) N-acetylcysteine, or (±) N-acetylcysteine.

In another aspect, the present invention provides compounds of Formula (I) wherein R₁ is hydrogen or acetyl; R₂, R₃, R₄, and R₅ are independently hydrogen, halo(C₁-C₆)alkyl, halogen, or phenyl, wherein the phenyl is optionally substituted with 1, 2, 3, 4, or 5 groups that are independently C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ₃Z₄, or (N₃Z₄)carbonyl; R₆ is formula (i); R₇ is (C₁-C₆)alkoxy, (C₁-C₆)alkyl, (C₁-C₆)alkylthio, hydroxy, or —NZ₉Z₁₀; R₉ is hydrogen or (C₁-C₆)alkyl; R₉ is (C₁-C₆)alkylcarbonyl; X₁ is O or S; L is (C₁-C₆)alkylene; and Z₃, Z₄, Z₉, and Z₁₀ are independently hydrogen, (C₁-C₆)alkyl, or (C₁-C₆)alkylcarbonyl; provided that Formula (I) does not encompass R₁ is hydrogen or acetyl; R₂, R₃, and R₅ are hydrogen; R₄ is hydrogen or 2,4-difluorophenyl; and R₆ is (L) N-acetylcysteine, (D) N-acetylcysteine, or (±) N-acetylcysteine.

In another aspect, the present invention provides compounds of Formula (I) wherein R₁ is hydrogen or acetyl; R₂, R₃, R₄, and R₅ are independently hydrogen or phenyl, wherein the phenyl is optionally substituted with 1 or 2 groups that are independently halo(C₁-C₆)alkoxy, halo(C₁-C₆)alkyl, or halogen; R₆ is formula (i); R₇ is (C₁-C₆)alkoxy or hydroxy; R₈ is hydrogen or (C₁-C₆)alkyl; R₉ is (C₁-C₆)alkylcarbonyl; X₁ is O or S; and L is (C₁-C₆)alkylene; provided that Formula (I) does not encompass R₁ is hydrogen or acetyl; R₂, R₃, and R₅ are hydrogen; R₄ is hydrogen or 2,4-difluorophenyl; and R₆ is (L) N-acetylcysteine, (D) N-acetylcysteine, or (±) N-acetylcysteine.

In another aspect, the present invention provides compounds of Formula (I) wherein R₁ is hydrogen or acetyl; R₂, R₃, R₄, and R₅ are independently hydrogen or phenyl, wherein the phenyl is optionally substituted with 1 or 2 halogen groups; R₆ is formula (i); R₇ is ethoxy, methoxy, or hydroxy; R₉ is hydrogen or methyl; R₉ is acetyl; X₁ is O or S; and L is CH₂; provided that Formula (I) does not encompass R₁ is hydrogen or acetyl; R₂, R₃, and R₅ are hydrogen; R₄ is hydrogen or 2,4-difluorophenyl; and R₆ is (L) N-acetylcysteine, (D) N-acetylcysteine, or (±) N-acetylcysteine.

In another aspect, the present invention provides compounds of Formula (I) wherein R₁ is hydrogen or acetyl; R₂, R₃, R₄, and R₅ are independently hydrogen, halo(C₁-C₆)alkyl, or halogen; R₆ is formula (i); R₇ is (C₁-C₆)alkoxy, (C₁-C₆)alkyl, (C₁-C₆)alkylthio, hydroxy, or —NZ₉Z₁₀; R₉ is hydrogen or (C₁-C₆)alkyl; R₉ is (C₁-C₆)alkylcarbonyl; X₁ is O or S; L is (C₁-C₆)alkylene; and Z₉, and Z₁₀ are independently hydrogen, (C₁-C₆)alkyl, or (C₁-C₆)alkylcarbonyl; provided that Formula (I) does not encompass R₁ is hydrogen or acetyl; R₂, R₃, R₄, and R₅ are hydrogen; and R₆ is (L) N-acetylcysteine, (D) N-acetylcysteine, or (±) N-acetylcysteine.

In another aspect, the present invention provides compounds of Formula (I) wherein R₁ is hydrogen or acetyl; R₂, R₃, R₄, and R₅ are independently hydrogen or halo(C₁-C₆)alkyl; R₆ is formula (i); R₇ is (C₁-C₆)alkoxy or hydroxy; R₉ is hydrogen or (C₁-C₆)alkyl; R₉ is (C₁-C₆)alkylcarbonyl; X₁ is O or S; and L is (C₁-C₆)alkylene; provided that Formula (I) does not encompass R₁ is hydrogen or acetyl; R₂, R₃, R₄, and R₅ are hydrogen; and R₆ is (L) N-acetylcysteine, (D) N-acetylcysteine, or (±) N-acetylcysteine.

In another aspect, the present invention provides compounds of Formula (I) wherein R₁ is hydrogen or acetyl; R₂, R₃, R₄, and R₅ are independently hydrogen or trifluormethyl; R₆ is formula (i); R₇ is ethoxy, methoxy, or hydroxy; R₉ is hydrogen or methyl; R₉ is acetyl; X₁ is O or S; and L is CH₂; provided that Formula (I) does not encompass R₁ is hydrogen or acetyl; R₂, R₃, R₄, and R₅ are hydrogen; and R₆ is (L) N-acetylcysteine, (D) N-acetylcysteine, or (±) N-acetylcysteine.

In another aspect, the present invention provides compounds of Formula (I) wherein R₁ is hydrogen or acetyl; one of R₂, R₃, R₄, and R₅ ais trifluormethyl and the rest are hydrogen; R₆ is formula (I); R₇ is hydroxy; R₉ is hydrogen; R₉ is acetyl; X₁ is S; and L is CH₂.

In another aspect, the present invention provides compounds of Formula (I) wherein R₁ is hydrogen or acetyl; R₂, R₃, R₄, and R₅ are independently hydrogen, halo(C₁-C₆)alkyl, or halogen; R₆ is —NZ₅Z₆; Z₅ is hydrogen; Z₆ is hydrogen, (C₁-C₆)alkyl, (C₁-C₆)alkylcarbonyl, phenyl, phenyl(CH₂)—, or phenyl(CH₂)₂—, wherein the phenyl is optionally substituted with 1, 2, 3, 4, or 5 groups that are independently (C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, —C₁₋₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ₇Z₈, or (NZ₇Z₈)carbonyl; and Z₇ and Z₈ are independently hydrogen, (C₁-C₆)alkyl, or (C₁-C₆)alkylcarbonyl

In another aspect, the present invention provides compounds of Formula (I) wherein R₁ is hydrogen or acetyl; R₂, R₃, R₄, and R₅ are independently hydrogen, halo(C₁-C₆)alkyl, or halogen; R₆ is —NZ₅Z₆; Z₅ is hydrogen; Z₆ is hydrogen, (C₁-C₆)alkyl, (C₁-C₆)alkylcarbonyl.

In another aspect, the present invention provides compounds of Formula (I) wherein R₁ is hydrogen or acetyl; R₂, R₃, R₄, and R₅ are independently hydrogen, trifluoromethyl, or Cl; R₆ is —NZ₅Z₆; Z₅ is hydrogen; and Z₆ is hydrogen.

In another aspect, the present invention provides compounds of Formula (I) wherein R₁ is hydrogen or acetyl; R₂, R₃, R₄, and R₅ are independently hydrogen, halo(C₁-C₆)alkyl, or halogen; R₆ is —NZ₅Z₆; Z₅ is hydrogen; Z₆ is phenyl, wherein the phenyl is optionally substituted with 1, 2, 3, 4, or 5 groups that are independently (C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ₇Z₈, or (NZ₇Z₈)carbonyl; and Z₇ and Z₈ are independently hydrogen, (C₁-C₆)alkyl, or (C₁-C₆)alkylcarbonyl

In another aspect, the present invention provides compounds of Formula (I) wherein R₁ is hydrogen or acetyl; R₂, R₃, R₄, and R₅ are independently hydrogen, halo(C₁-C₆)alkyl, or halogen; R₆ is —NZ₅Z₆; Z₅ is hydrogen; Z₆ is phenyl, wherein the phenyl is optionally substituted with 1 or 2 groups that are independently halo(C₁-C₆)alkyl or halogen.

In another aspect, the present invention provides compounds of Formula (I) wherein R₁ is hydrogen or acetyl; R₂, R₃, R₄, and R₅ are independently hydrogen, trifluoromethyl, or Cl; R₆ is —NZ₅Z₆; Z₅ is hydrogen; Z₆ is phenyl, wherein the phenyl is optionally substituted with 1 or 2 groups that are independently trifluoromethyll or Cl.

Representative compounds of Formula (I) include, but are not limited to, the compounds shown below, wherein R₁ is hydrogen or acetyl.

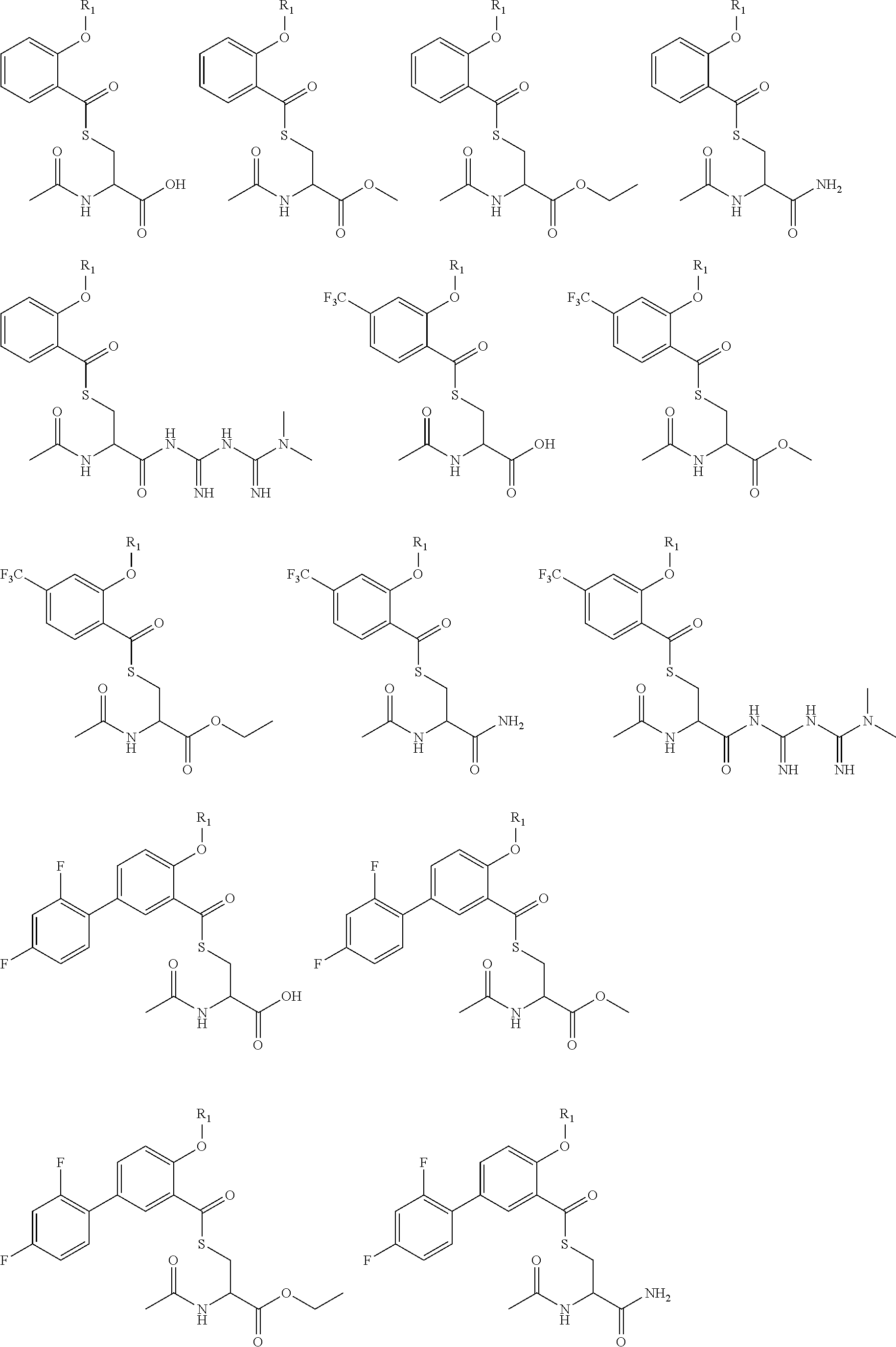

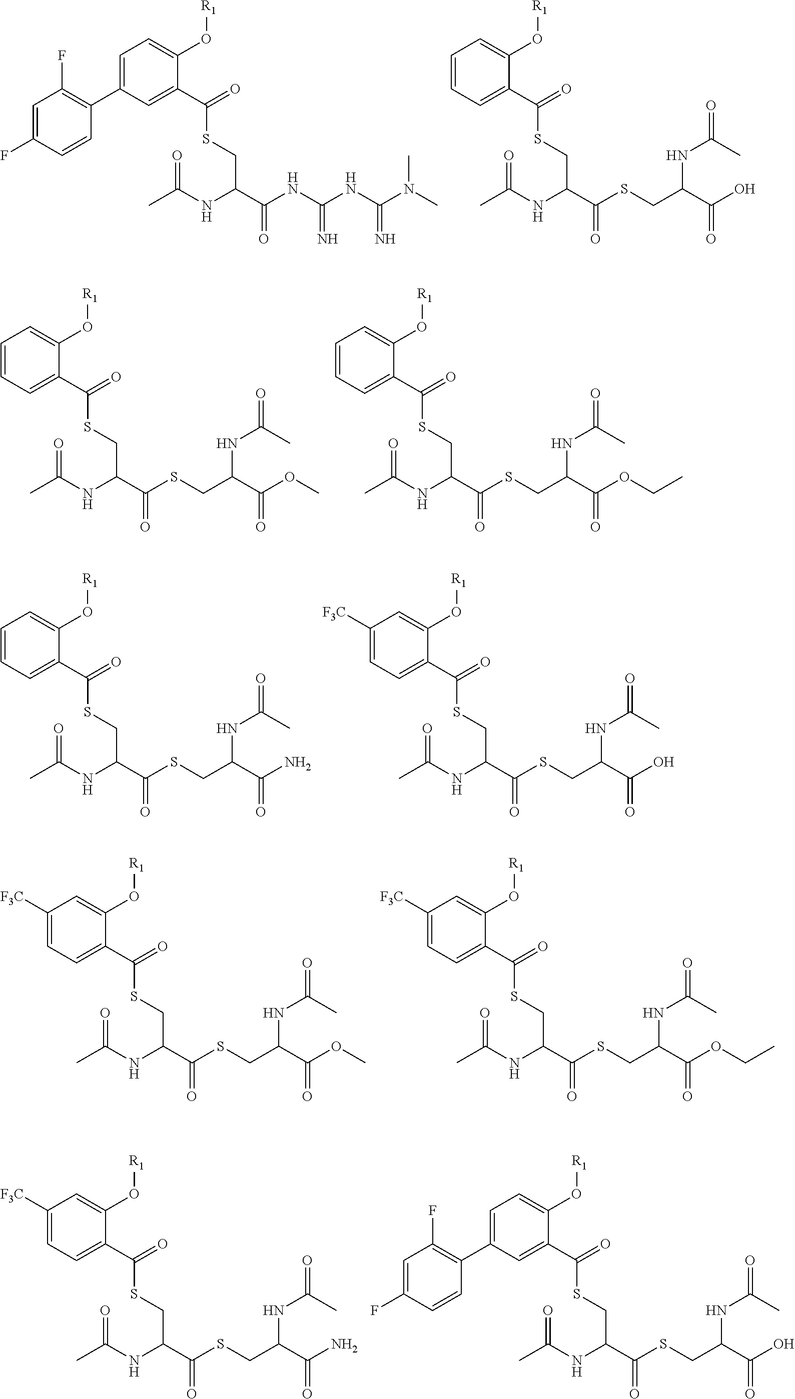

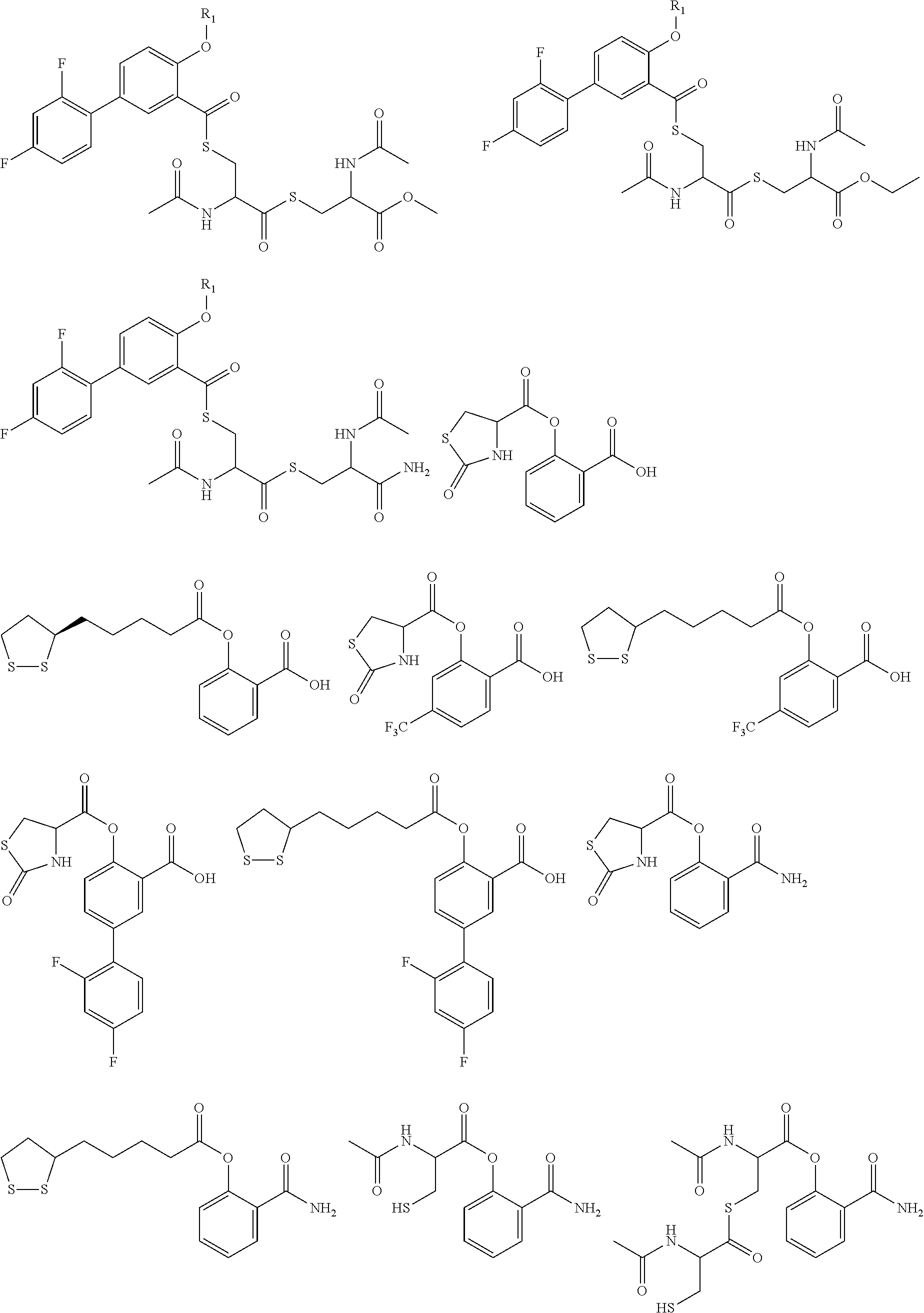

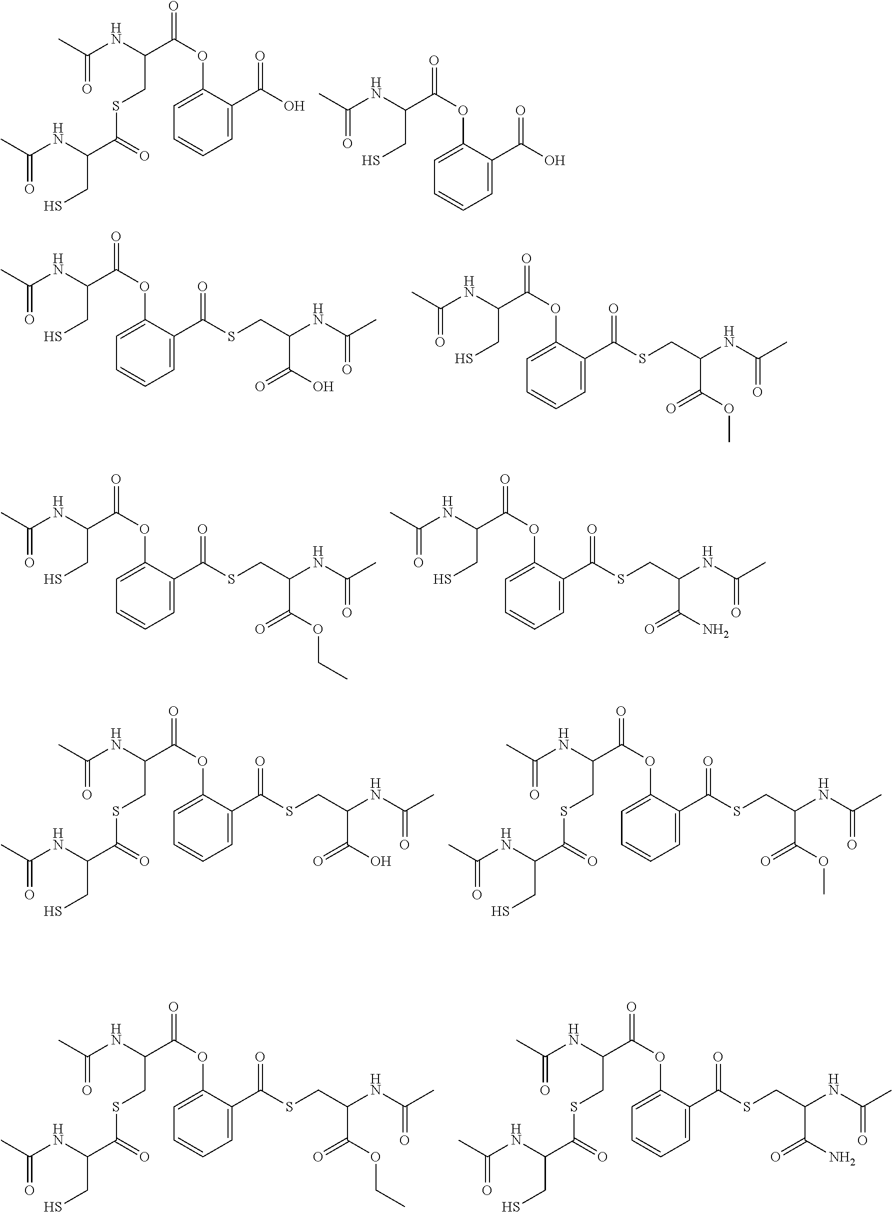

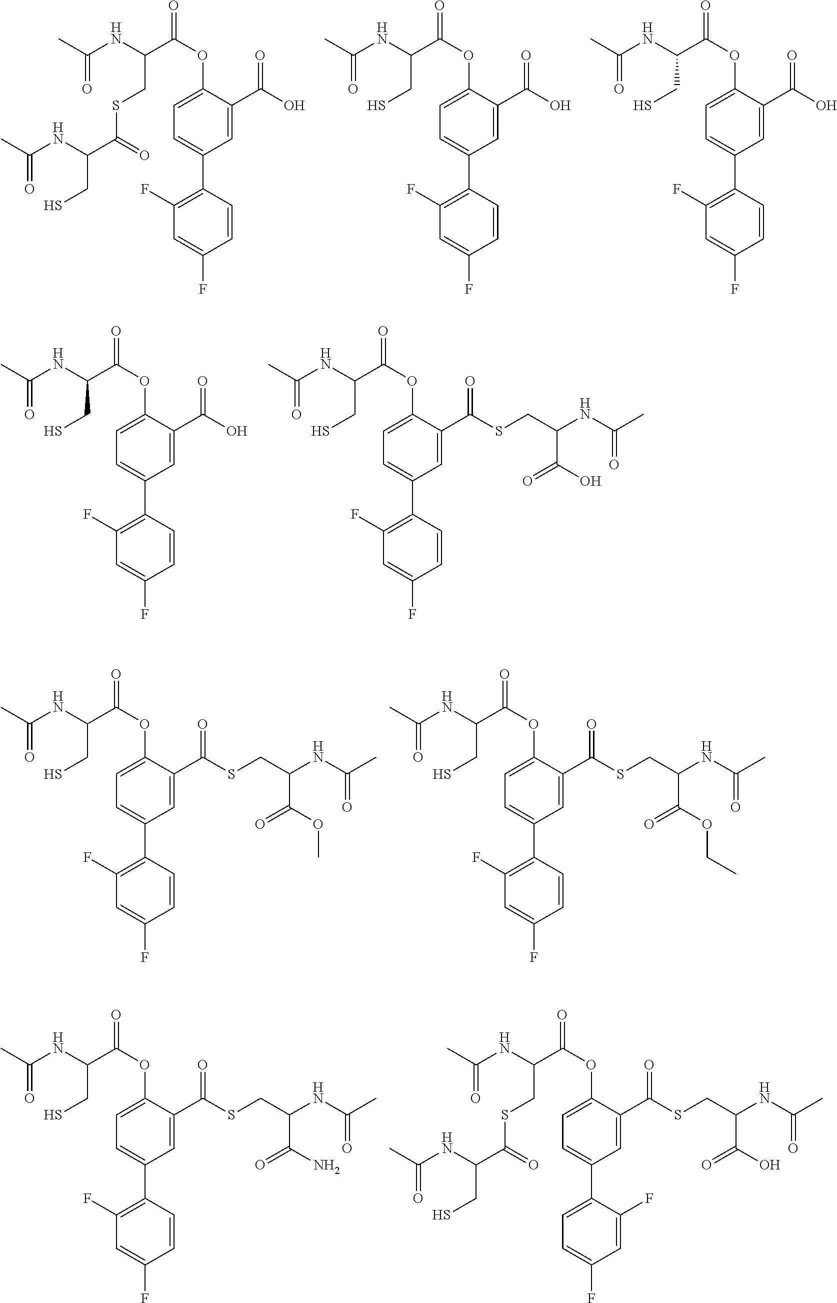

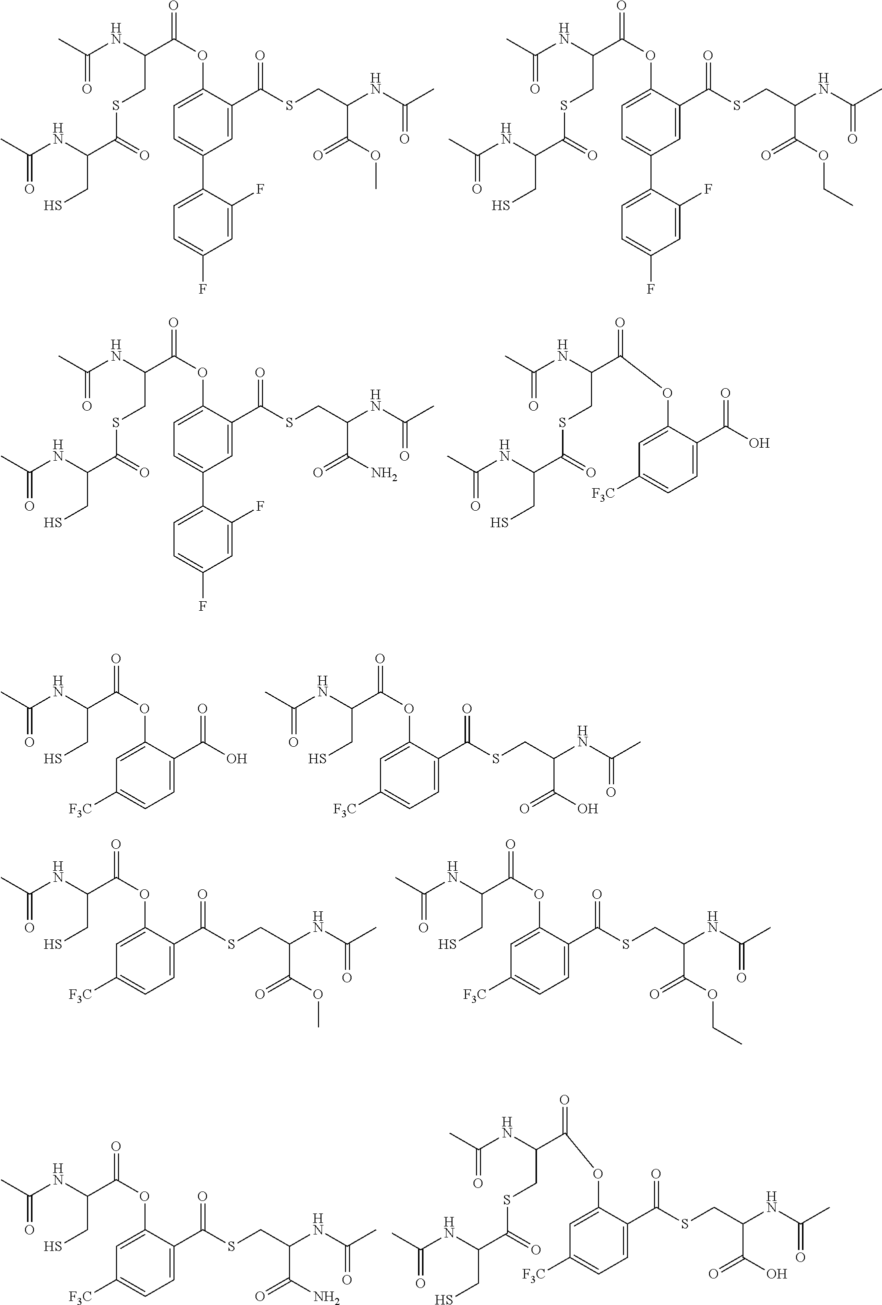

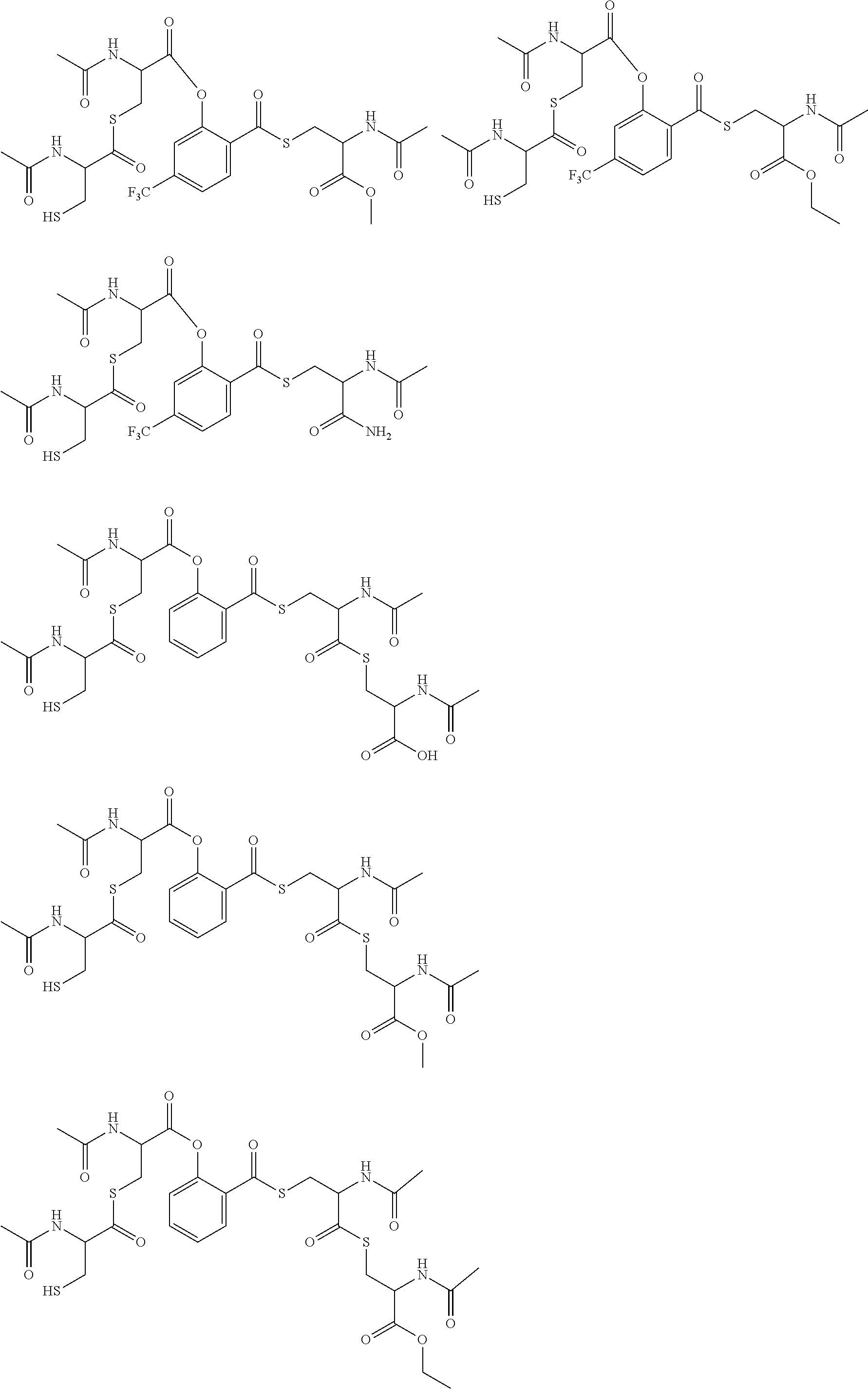

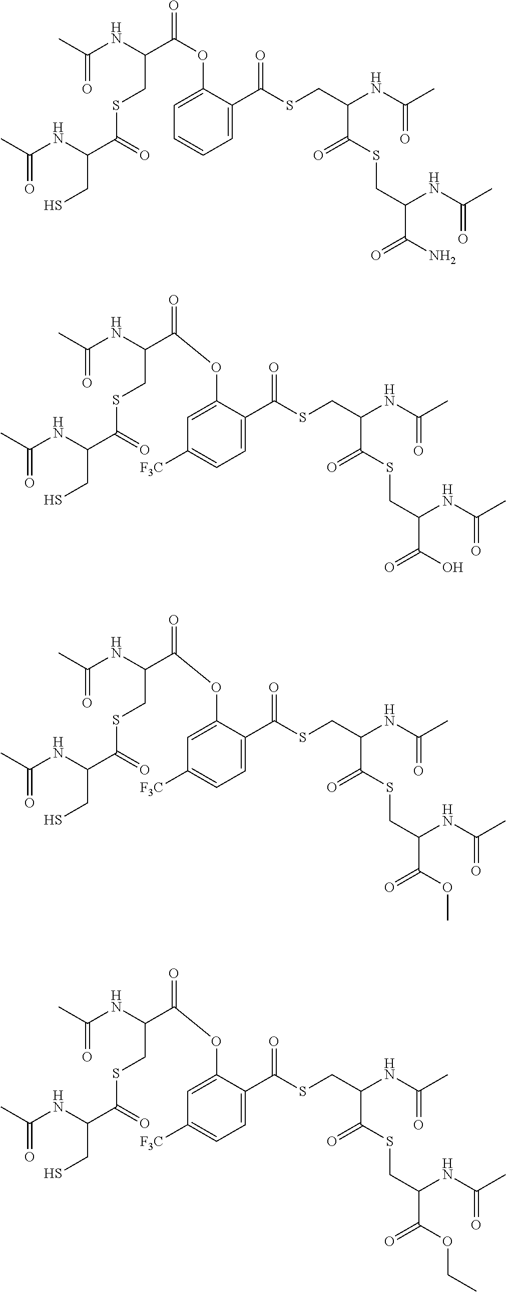

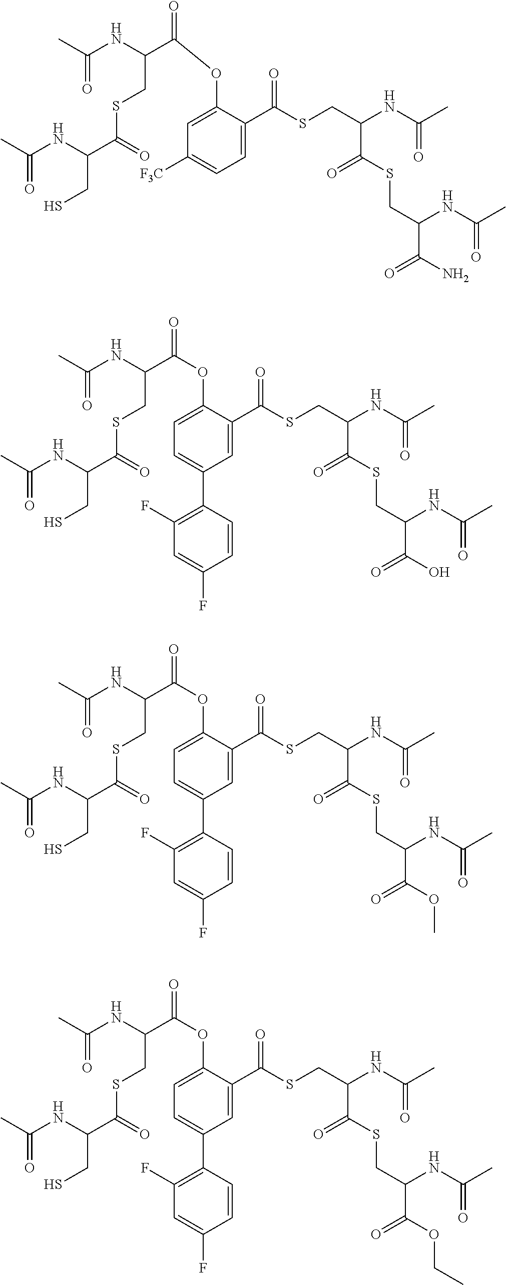

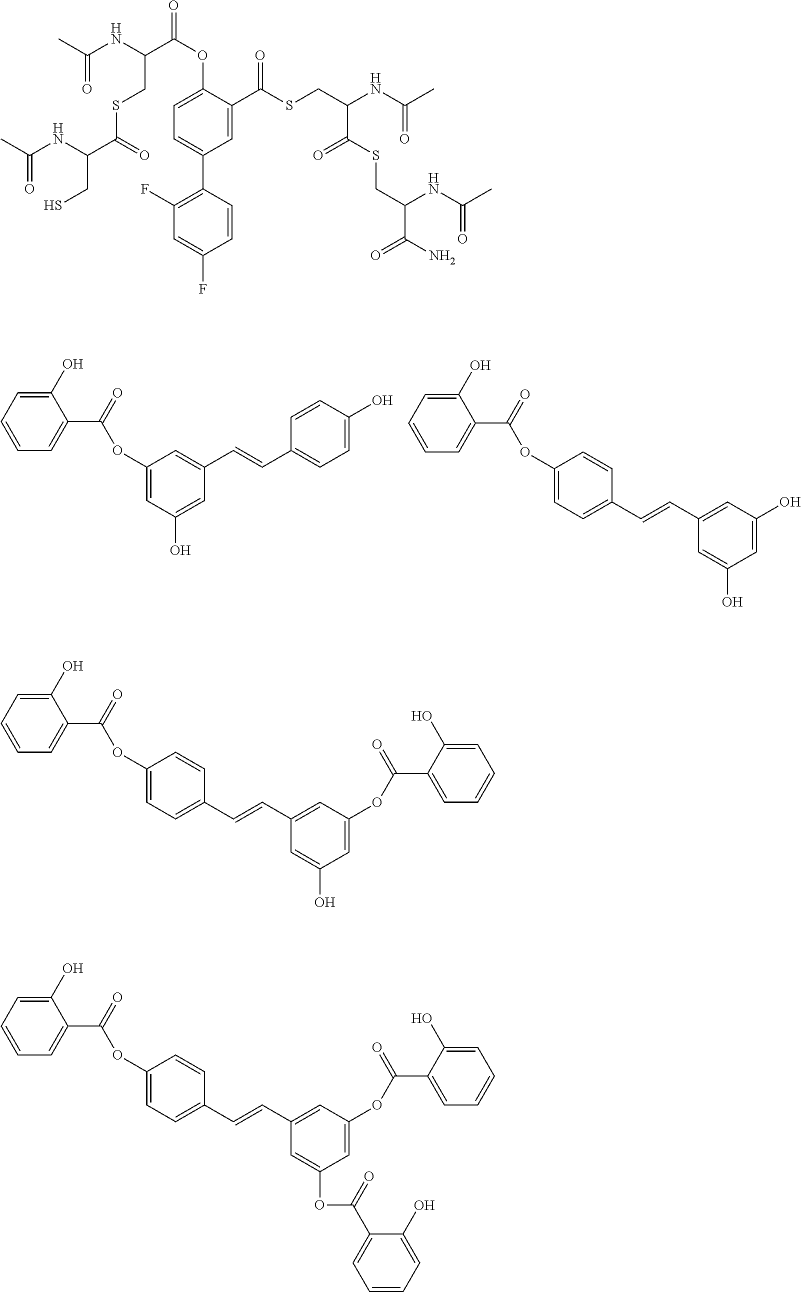

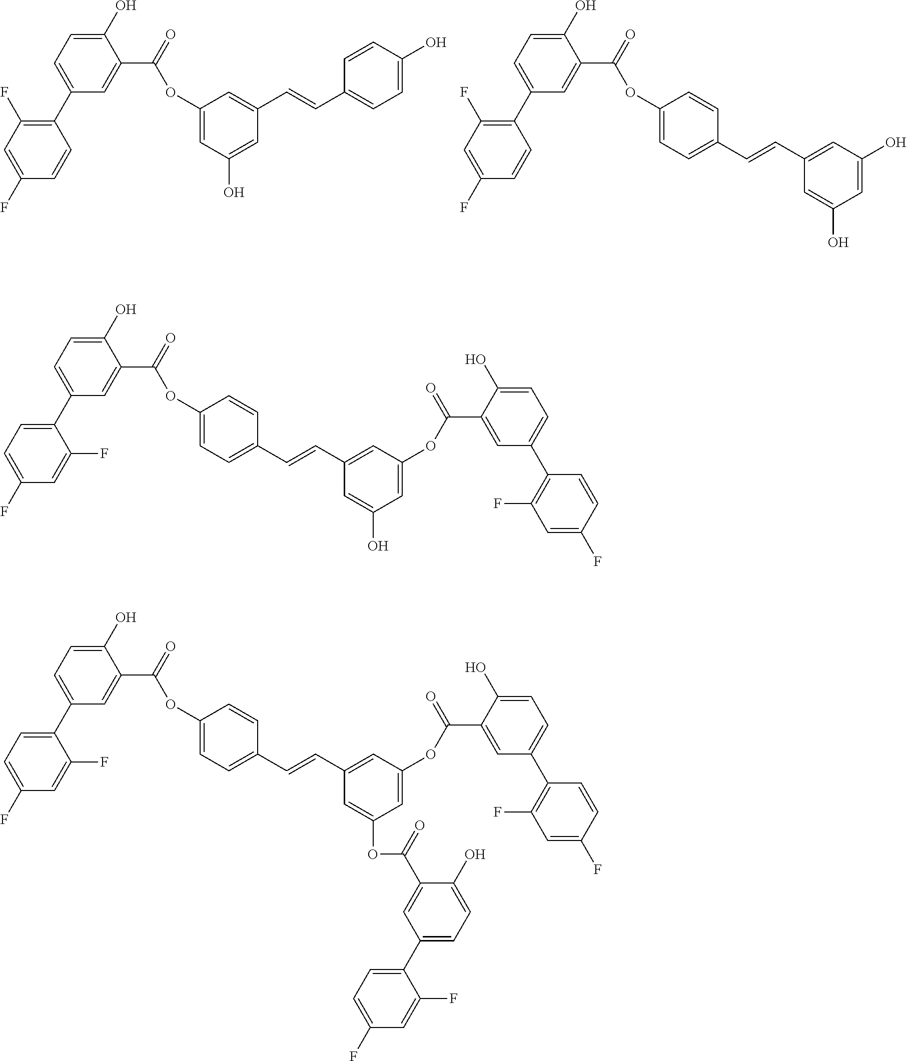 [SMILES: O=C(Oc1cc(O)cc(/C=C/c2ccc(O)cc2)c1)c1cc(-c2ccc(F)cc2F)ccc1O ; O=C(Oc1ccc(/C=C/c2cc(O)cc(O)c2)cc1)c1cc(-c2ccc(F)cc2F)ccc1O ; O=C(Oc1ccc(/C=C/c2cc(O)cc(OC(=O)c3cc(-c4ccc(F)cc4F)ccc3O)c2)cc1)c1cc(-c2ccc(F)cc2F)ccc1O ; O=C(Oc1ccc(/C=C/c2cc(OC(=O)c3cc(-c4ccc(F)cc4F)ccc3O)cc(OC(=O)c3cc(-c4ccc(F)cc4F)ccc3O)c2)cc1)c1cc(-c2ccc(F)cc2F)ccc1O]

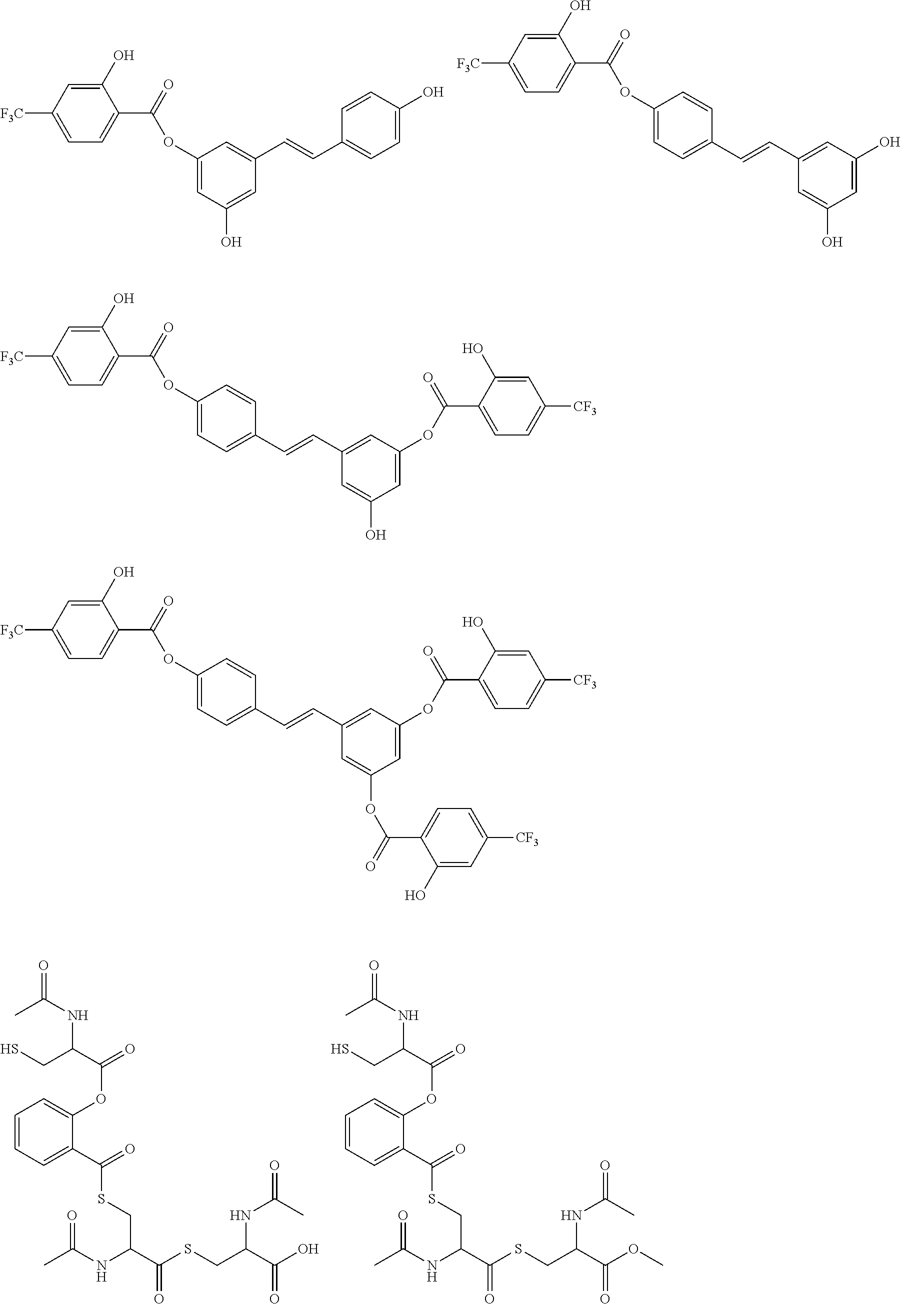 [SMILES: O=C(Oc1cc(O)cc(/C=C/c2ccc(O)cc2)c1)c1ccc(C(F)(F)F)cc1O ; O=C(Oc1ccc(/C=C/c2cc(O)cc(O)c2)cc1)c1ccc(C(F)(F)F)cc1O ; O=C(Oc1ccc(/C=C/c2cc(O)cc(OC(=O)c3ccc(C(F)(F)F)cc3O)c2)cc1)c1ccc(C(F)(F)F)cc1O ; O=C(Oc1ccc(/C=C/c2cc(OC(=O)c3ccc(C(F)(F)F)cc3O)cc(OC(=O)c3ccc(C(F)(F)F)cc3O)c2)cc1)c1ccc(C(F)(F)F)cc1O ; CC(=O)NC(CSC(=O)C(CSC(=O)c1ccccc1OC(=O)C(CS)NC(C)=O)NC(C)=O)C(=O)O ; COC(=O)C(CSC(=O)C(CSC(=O)c1ccccc1OC(=O)C(CS)NC(C)=O)NC(C)=O)NC(C)=O]

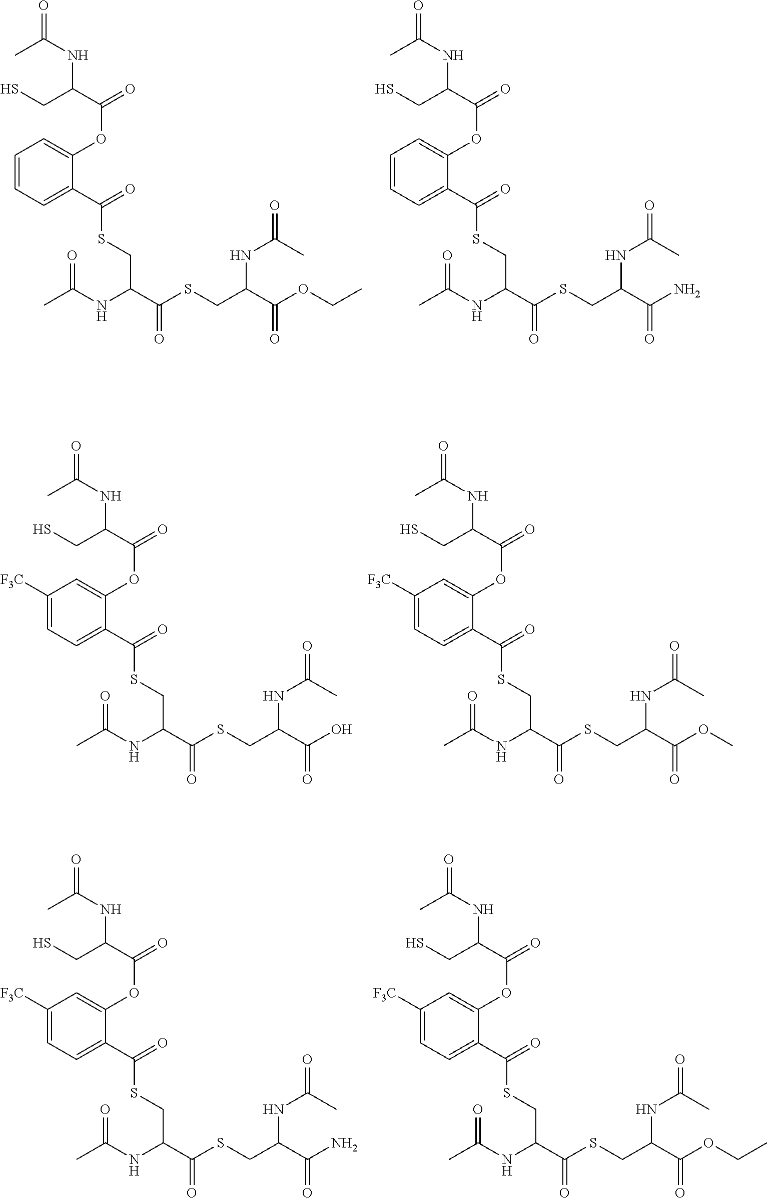 [SMILES: CCOC(=O)C(CSC(=O)C(CSC(=O)c1ccccc1OC(=O)C(CS)NC(C)=O)NC(C)=O)NC(C)=O ; CC(=O)NC(CSC(=O)C(CSC(=O)c1ccccc1OC(=O)C(CS)NC(C)=O)NC(C)=O)C(N)=O ; CC(=O)NC(CSC(=O)C(CSC(=O)c1ccc(C(F)(F)F)cc1OC(=O)C(CS)NC(C)=O)NC(C)=O)C(=O)O ; COC(=O)C(CSC(=O)C(CSC(=O)c1ccc(C(F)(F)F)cc1OC(=O)C(CS)NC(C)=O)NC(C)=O)NC(C)=O ; CC(=O)NC(CSC(=O)C(CSC(=O)c1ccc(C(F)(F)F)cc1OC(=O)C(CS)NC(C)=O)NC(C)=O)C(N)=O ; CCOC(=O)C(CSC(=O)C(CSC(=O)c1ccc(C(F)(F)F)cc1OC(=O)C(CS)NC(C)=O)NC(C)=O)NC(C)=O]

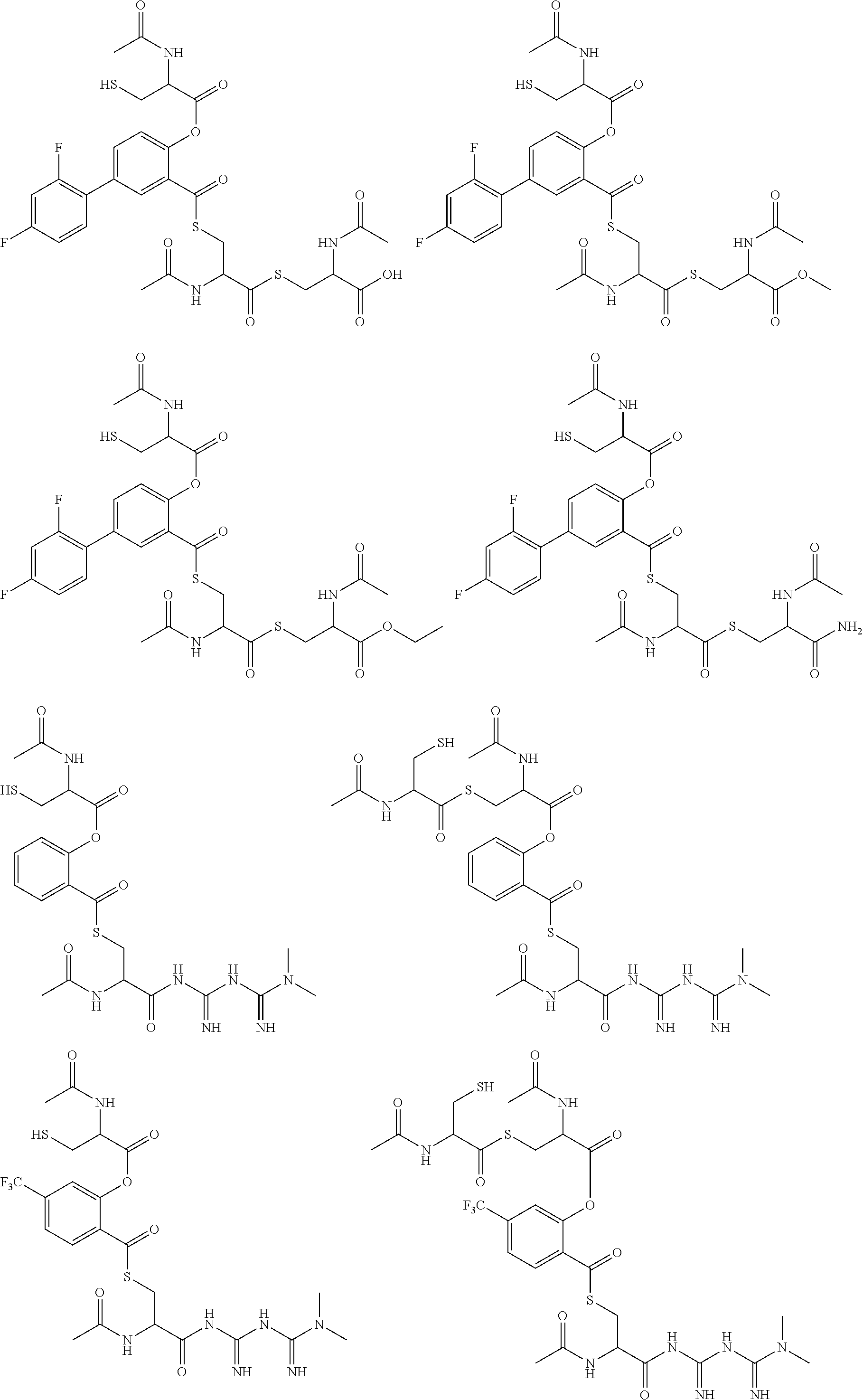

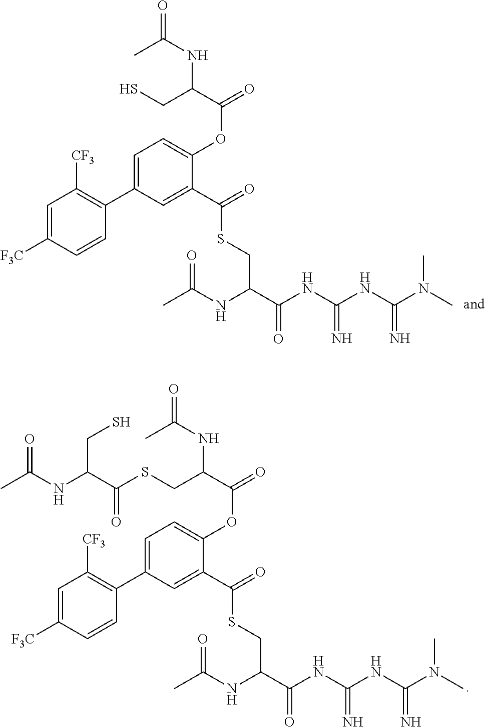

In another aspect, the present invention provides methods for treating atherosclerosis, neuropathy, nephropathy, retinopathy, inflammatory disorders, cardiovascular diseases, and metabolic disorders in a mammal or patient comprising administering to the mammal or patient in need of such treatment a therapeutically effective amount of a compound of Formula (I), shown above, or a pharmaceutically acceptable salt thereof.

In certain embodiments, the inventive methods include treating dyslipidemia, insulin resistance, β-cell dysfunction, hyperglycemia, metabolic syndrome, and any form of diabetes mellitus including type I and type II diabetes, in a patient comprising administering to the patient in need of such treatment a therapeutically effective amount of a compound of Formula (I), shown above, or a pharmaceutically acceptable salt thereof.

In certain embodiments, the present invention provides methods for reducing advanced glycated end products and/or lipid peroxidation including, but not limited to, oxidation of low-density lipoproteins in a mammal or patient comprising administering to the mammal or patient in need of such treatment a therapeutically effective amount of a compound of Formula (I), as shown above, or a pharmaceutically acceptable salt thereof.

In certain embodiments, the inventive methods include treating dyslipidemia, insulin resistance, β-cell dysfunction, hyperglycemia, metabolic syndrome, and any form of diabetes mellitus including type I and type II diabetes, in a patient comprising administering to the patient in need of such treatment a therapeutically effective amount of a pharmaceutical composition comprising at least one pharmaceutically acceptable carrier and a compound of Formula (I), shown above, or a pharmaceutically acceptable salt thereof.

In certain embodiments, the present invention provides methods for reducing advanced glycated end products and lipid peroxidation including, but not limited to, oxidation of low-density lipoproteins, in a patient comprising administering to the patient in need of such treatment a therapeutically effective amount of a pharmaceutical composition comprising at least one pharmaceutically acceptable carrier and a compound of Formula (I), as shown above, or a pharmaceutically acceptable salt thereof.

In another aspect, the present invention provides compounds of Formula (II)

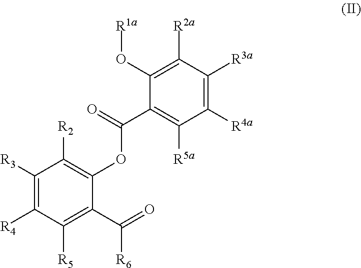

wherein R₂, R₃, R₄, R₅, R₆, R₁ₐ, R₂ₐ, R₃ₐ, R₄ₐ, and R₅ₐ are as defined in Formula (I) of the Summary section.

In another aspect, the present invention provides compounds of Formula (II) wherein R₂, R₃, R₄, R₅, are independently hydrogen, trifluoromethyl, or 2,4-difluorophenyl; R₆ is as defined in Formula (I) of the Summary section; R₁ₐ is hydrogen or acetyl; and R₂ₐ, R₃ₐ, R₄ₐ, and R₅ₐ, are independently hydrogen, trifluoromethyl, or 2,4-difluorophenyl.

In another aspect, the present invention provides compounds of Formula (II) wherein R₂, R₃, R₄, R₅, are independently hydrogen, trifluoromethyl, or 2,4-difluorophenyl; R₆ is N-acetylcysteine, (L) N-acetylcysteine, or (D) N-acetylcysteine; R₁ₐ is hydrogen or acetyl; and one of R₂ₐ, R₃ₐ, R₄ₐ, and R₅ₐ is C(O)—R₆ₐ and the rest are hydrogen; and R₆ₐ is as defined in Formula (I).

Representative compounds of Formula (II) include, but are not limited to, the compounds shown below, wherein R₁ₐ is hydrogen or acetyl.

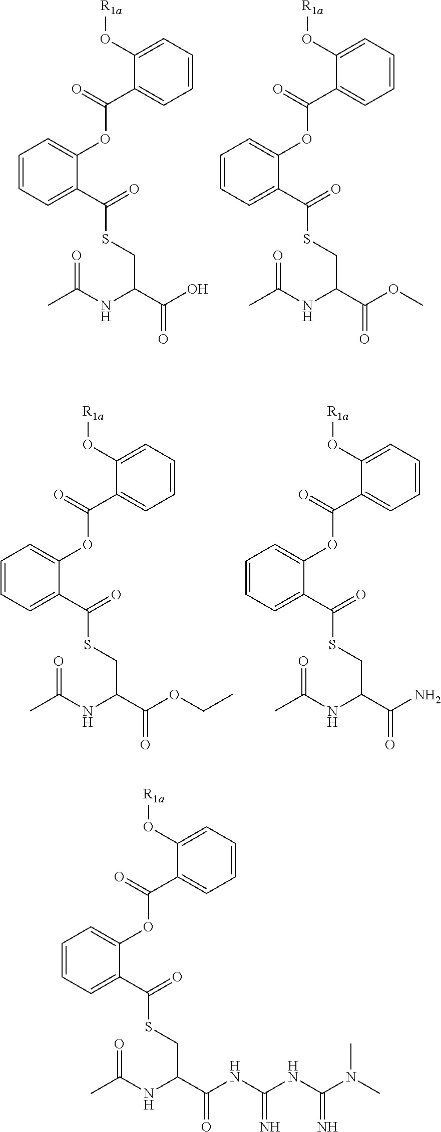

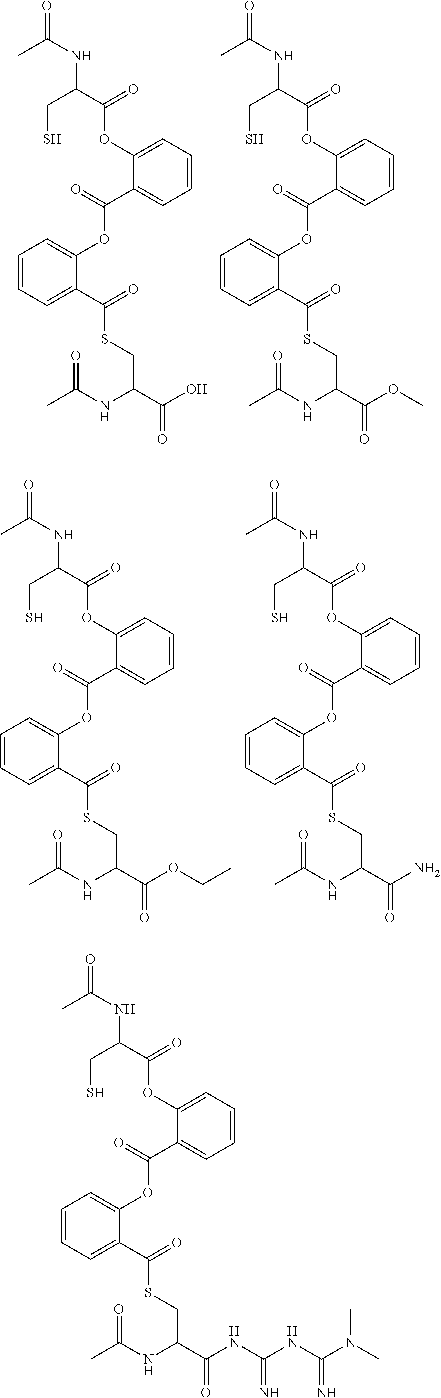

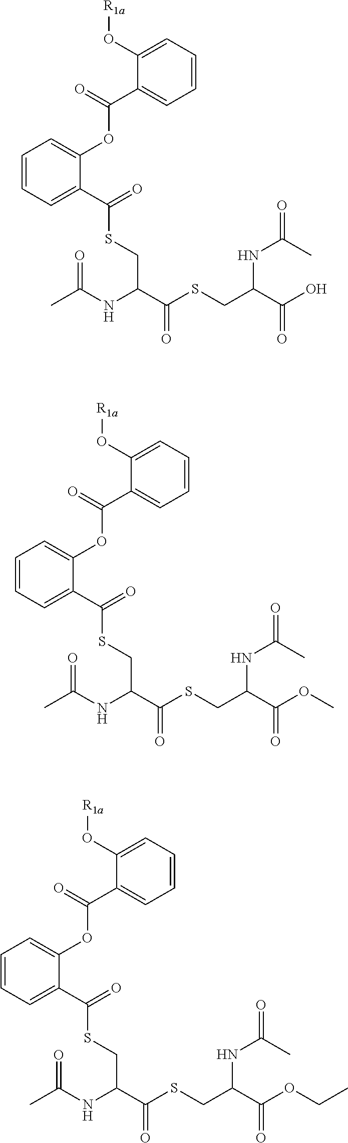

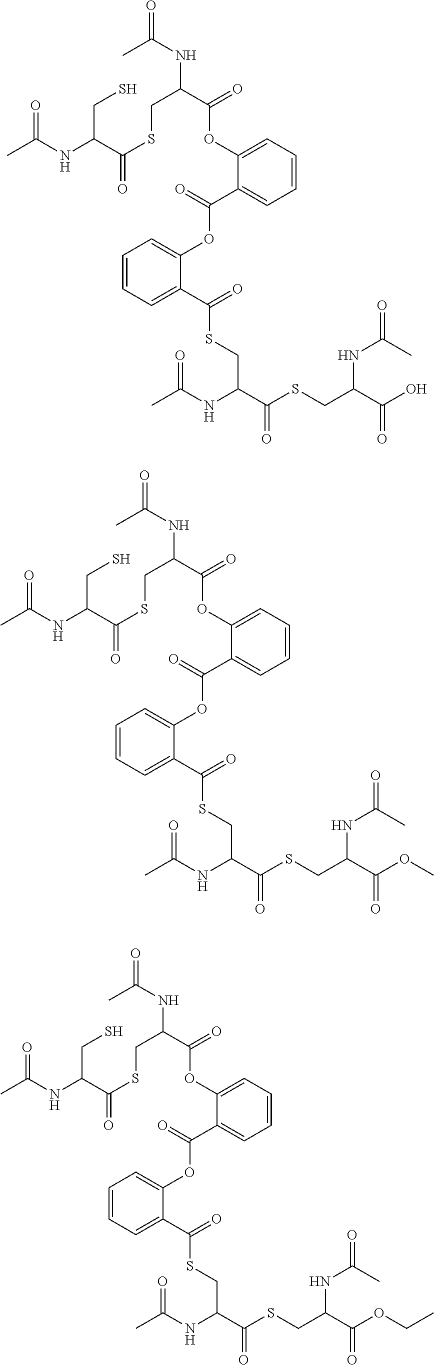

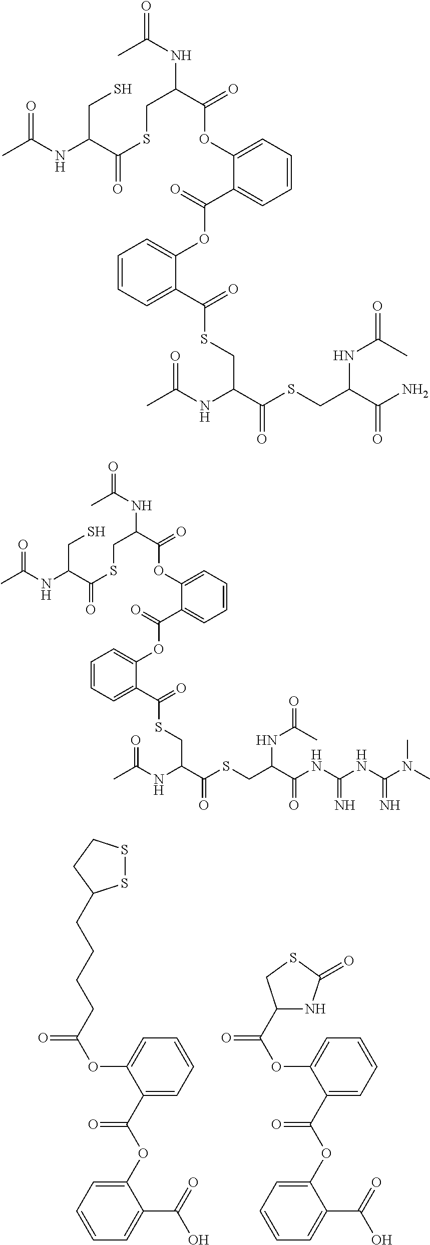

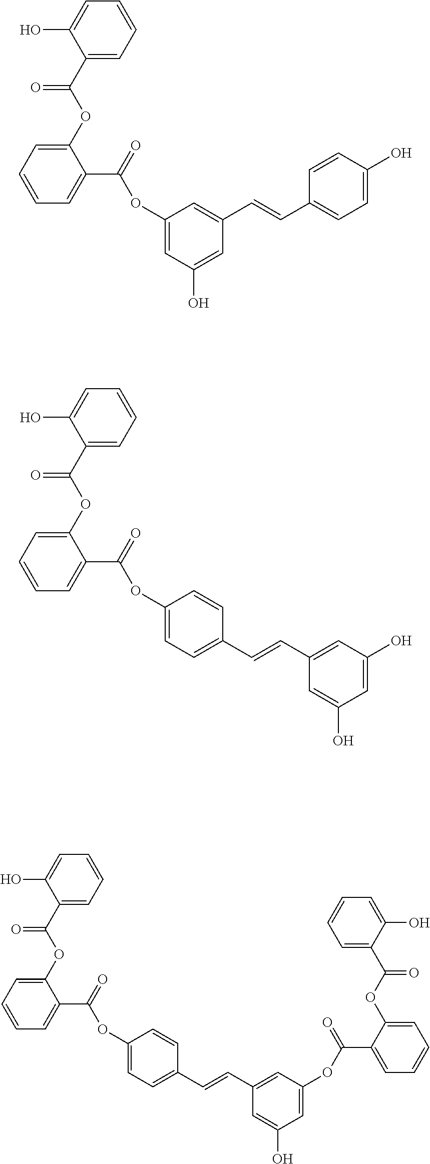 [SMILES: O=C(Oc1ccccc1C(=O)Oc1cc(O)cc(/C=C/c2ccc(O)cc2)c1)c1ccccc1O ; O=C(Oc1ccccc1C(=O)Oc1ccc(/C=C/c2cc(O)cc(O)c2)cc1)c1ccccc1O ; O=C(Oc1ccccc1C(=O)Oc1ccc(/C=C/c2cc(O)cc(OC(=O)c3ccccc3OC(=O)c3ccccc3O)c2)cc1)c1ccccc1O]

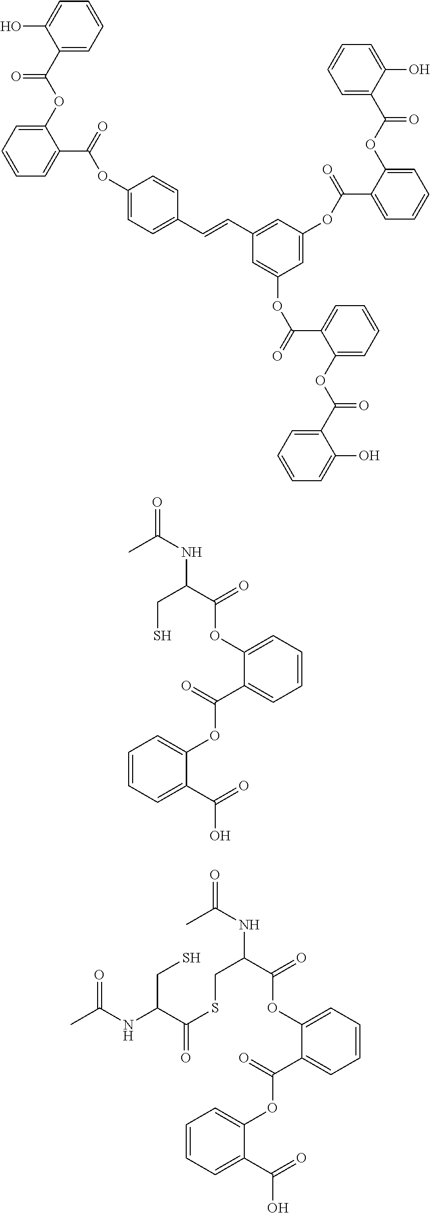

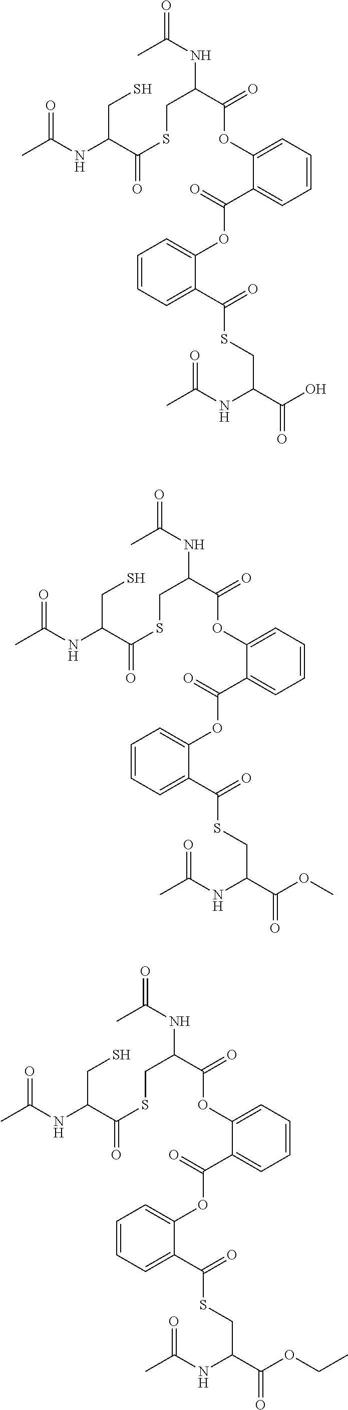

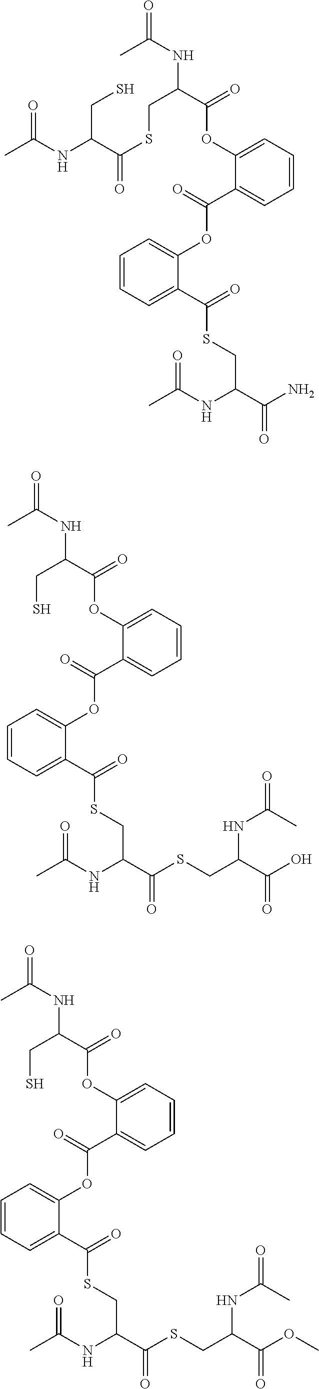 [SMILES: CC(=O)NC(CSC(=O)c1ccccc1OC(=O)c1ccccc1OC(=O)C(CSC(=O)C(CS)NC(C)=O)NC(C)=O)C(N)=O ; CC(=O)NC(CSC(=O)C(CSC(=O)c1ccccc1OC(=O)c1ccccc1OC(=O)C(CS)NC(C)=O)NC(C)=O)C(=O)O ; COC(=O)C(CSC(=O)C(CSC(=O)c1ccccc1OC(=O)c1ccccc1OC(=O)C(CS)NC(C)=O)NC(C)=O)NC(C)=O]

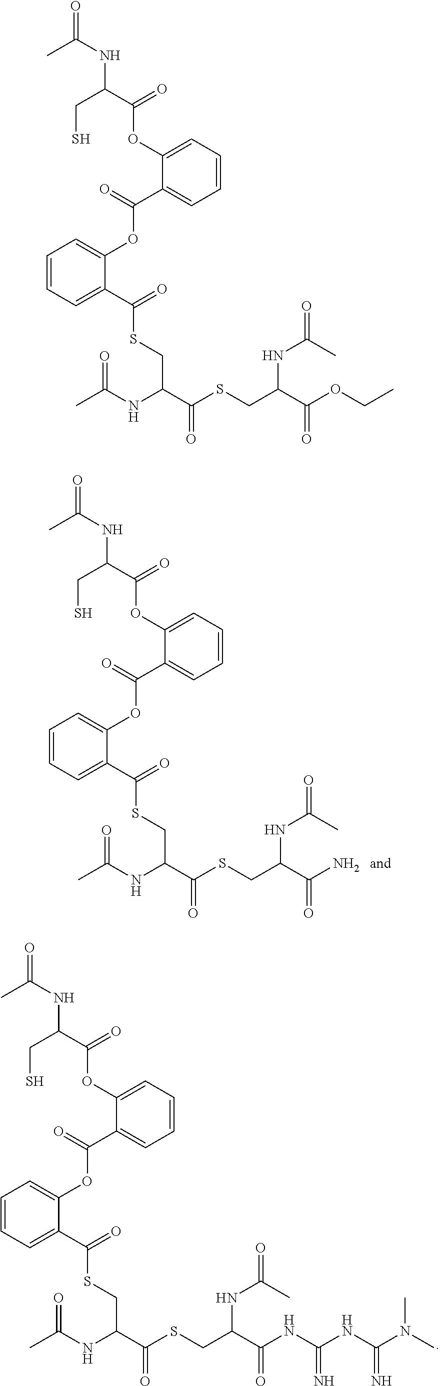 [SMILES: CCOC(=O)C(CSC(=O)C(CSC(=O)c1ccccc1OC(=O)c1ccccc1OC(=O)C(CS)NC(C)=O)NC(C)=O)NC(C)=O ; CC(=O)NC(CSC(=O)C(CSC(=O)c1ccccc1OC(=O)c1ccccc1OC(=O)C(CS)NC(C)=O)NC(C)=O)C(N)=O ; "and" ; CC(=O)NC(CSC(=O)C(CSC(=O)c1ccccc1OC(=O)c1ccccc1OC(=O)C(CS)NC(C)=O)NC(C)=O)C(=O)NC(=N)NC(=N)N(C)C]

In another aspect, the present invention provides methods for treating atherosclerosis, neuropathy, nephropathy, retinopathy, inflammatory disorders, cardiovascular diseases, and metabolic disorders in a mammal or patient comprising administering to the mammal or patient in need of such treatment a therapeutically effective amount of a compound of Formula (II), as shown above, or a pharmaceutically acceptable salt thereof.

In certain embodiments, the inventive methods include treating dyslipidemia, insulin resistance, β-cell dysfunction, hyperglycemia, metabolic syndrome, and any form of diabetes mellitus including type I and type II diabetes, in a patient comprising administering to the patient in need of such treatment a therapeutically effective amount of a compound of Formula (II), as shown above, or a pharmaceutically acceptable salt thereof.

In certain embodiments, the present invention provides methods for reducing advanced glycated end products and/or lipid peroxidation including, but not limited to, oxidation of low-density lipoproteins in a mammal or patient comprising administering to the mammal or patient in need of such treatment a therapeutically effective amount of a compound of Formula (II), as shown above, or a pharmaceutically acceptable salt thereof.

In certain embodiments, the inventive methods include treating dyslipidemia, insulin resistance, β-cell dysfunction, hyperglycemia, metabolic syndrome, and any form of diabetes mellitus including type I and type II diabetes, in a patient comprising administering to the patient in need of such treatment a therapeutically effective amount of a pharmaceutical composition comprising at least one pharmaceutically acceptable carrier and a compound of Formula (II), as shown above, or a pharmaceutically acceptable salt thereof.

In certain embodiments, the present invention provides methods for reducing advanced glycated end products and lipid peroxidation including, but not limited to, oxidation of low-density lipoproteins, in a patient comprising administering to the patient in need of such treatment a therapeutically effective amount of a pharmaceutical composition comprising at least one pharmaceutically acceptable carrier and a compound of Formula (II), as shown above, or a pharmaceutically acceptable salt thereof.

In another aspect, the present invention provides compounds of Formula (III)

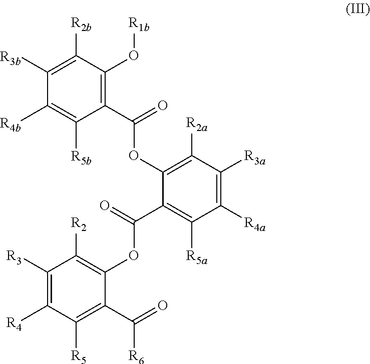

wherein R₂, R₃, R₄, R₅, R₆, R₂ₐ, R₃ₐ, R₄ₐ, R₅ₐ, R_{1b}, R_{2b}, R_{3b}, R_{4b}, and R_{5b} are as defined in Formula (I) of the Summary section.

In another aspect, the present invention provides compounds of Formula (III) wherein R₂, R₃, R₄, R₅, are independently hydrogen, trifluoromethyl, or 2,4-difluorophenyl; R₆ is (L) N-acetylcysteine; R₂ₐ, R₃ₐ, R₄ₐ, and R₅ₐ, are independently hydrogen, trifluoromethyl, or 2,4-difluorophenyl; R_{1b} is hydrogen or acetyl; and.

Representative compounds of Formula (III) include, but are not limited to, the compounds shown below, wherein R_{1b} is hydrogen or acetyl.

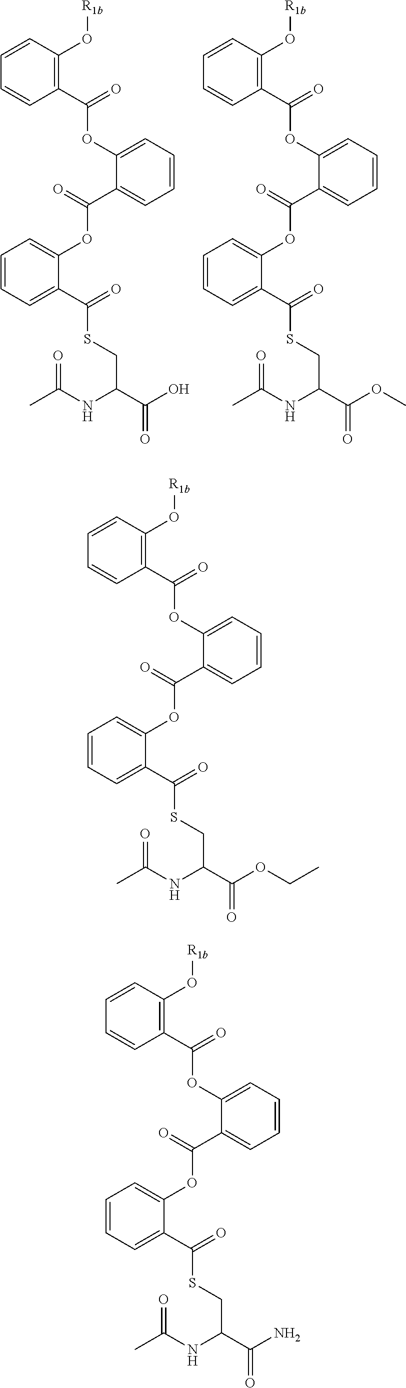

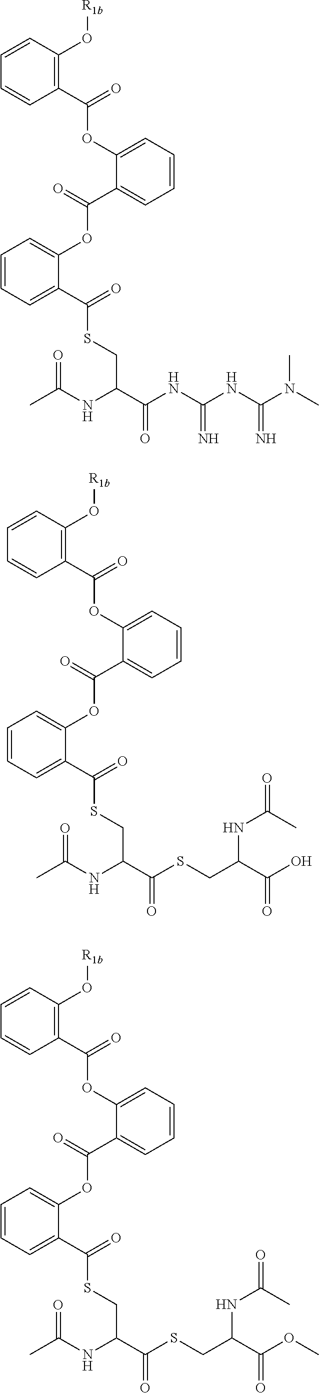

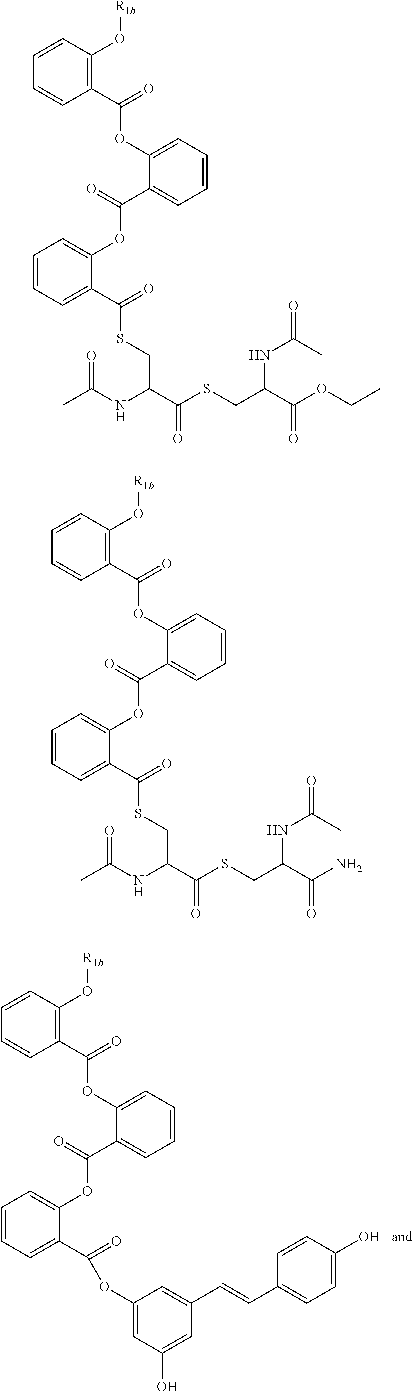

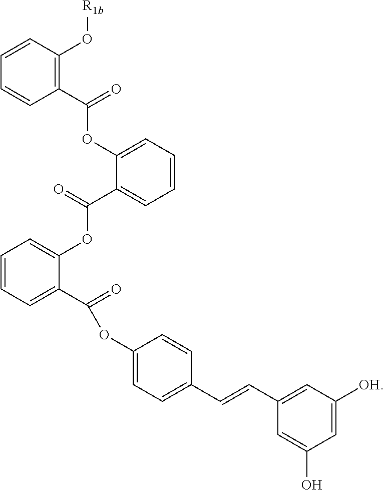

In another aspect, the present invention provides methods for treating atherosclerosis, neuropathy, nephropathy, retinopathy, inflammatory disorders, cardiovascular diseases, and metabolic disorders in a mammal or patient comprising administering to the mammal or patient in need of such treatment a therapeutically effective amount of a compound of Formula (III), as shown above, or a pharmaceutically acceptable salt thereof.

In certain embodiments, the inventive methods include treating dyslipidemia, insulin resistance, β-cell dysfunction, hyperglycemia, metabolic syndrome, and any form of diabetes mellitus including type I and type II diabetes, in a patient comprising administering to the patient in need of such treatment a therapeutically effective amount of a compound of Formula (III), as shown above, or a pharmaceutically acceptable salt thereof.

In certain embodiments, the present invention provides methods for reducing advanced glycated end products and/or lipid peroxidation including, but not limited to, oxidation of low-density lipoproteins in a mammal or patient comprising administering to the mammal or patient in need of such treatment a therapeutically effective amount of a compound of Formula (III), as shown above, or a pharmaceutically acceptable salt thereof.

In certain embodiments, the inventive methods include treating dyslipidemia, insulin resistance, β-cell dysfunction, hyperglycemia, metabolic syndrome, and any form of diabetes mellitus including type I and type II diabetes, in a patient comprising administering to the patient in need of such treatment a therapeutically effective amount of a pharmaceutical composition comprising at least one pharmaceutically acceptable carrier and a compound of Formula (III), as shown above, or a pharmaceutically acceptable salt thereof.

In certain embodiments, the present invention provides methods for reducing advanced glycated end products and lipid peroxidation including, but not limited to, oxidation of low-density lipoproteins, in a patient comprising administering to the patient in need of such treatment a therapeutically effective amount of a pharmaceutical composition comprising at least one pharmaceutically acceptable carrier and a compound of Formula (III), as shown above, or a pharmaceutically acceptable salt thereof.

In another aspect, the present invention provides compounds of Formula (IV)

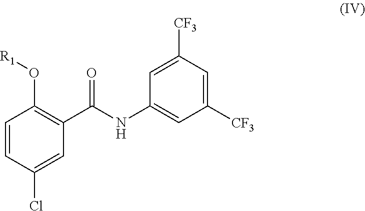

wherein R₁ is hydrogen, (C₁-C₆)alkylcarbonyl,

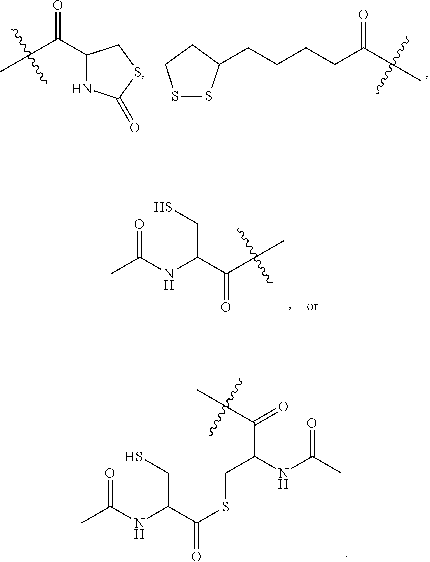

Representative compounds of Formula (IV) include the compounds shown below, wherein R₁ is hydrogen or acetyl.

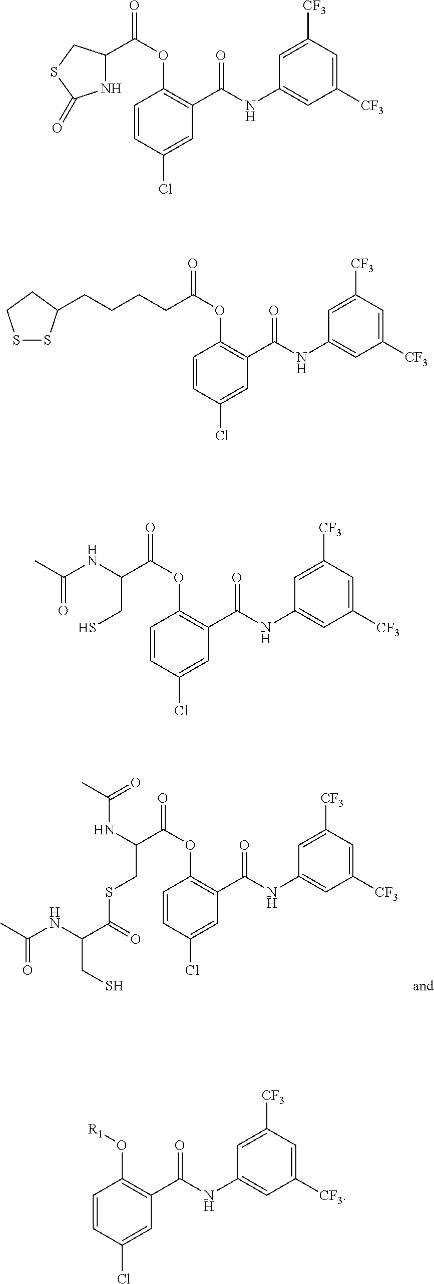

In another aspect, the present invention provides methods for treating atherosclerosis, neuropathy, nephropathy, retinopathy, inflammatory disorders, cardiovascular diseases, and metabolic disorders in a mammal or patient comprising administering to the mammal or patient in need of such treatment a therapeutically effective amount of a compound of Formula (IV), as shown above, or a pharmaceutically acceptable salt thereof.

In certain embodiments, the inventive methods include treating dyslipidemia, insulin resistance, β-cell dysfunction, hyperglycemia, metabolic syndrome, and any form of diabetes mellitus including type I and type II diabetes, in a patient comprising administering to the patient in need of such treatment a therapeutically effective amount of a compound of Formula (IV), as shown above, or a pharmaceutically acceptable salt thereof.

In certain embodiments, the present invention provides methods for reducing advanced glycated end products and/or lipid peroxidation including, but not limited to, oxidation of low-density lipoproteins in a mammal or patient comprising administering to the mammal or patient in need of such treatment a therapeutically effective amount of a compound of Formula (IV), as shown above, or a pharmaceutically acceptable salt thereof.

In certain embodiments, the inventive methods include treating dyslipidemia, insulin resistance, β-cell dysfunction, hyperglycemia, metabolic syndrome, and any form of diabetes mellitus including type I and type II diabetes, in a patient comprising administering to the patient in need of such treatment a therapeutically effective amount of a pharmaceutical composition comprising at least one pharmaceutically acceptable carrier and a compound of Formula (IV), or a pharmaceutically acceptable salt thereof.

In certain embodiments, the present invention provides methods for reducing advanced glycated end products and lipid peroxidation including, but not limited to, oxidation of low-density lipoproteins, in a patient comprising administering to the patient in need of such treatment a therapeutically effective amount of a pharmaceutical composition comprising at least one pharmaceutically acceptable carrier and a compound of Formula (IV), as shown above, or a pharmaceutically acceptable salt thereof, wherein

In another aspect, the present invention provides compounds of Formula (V)

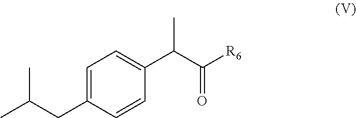

wherein R₆ is

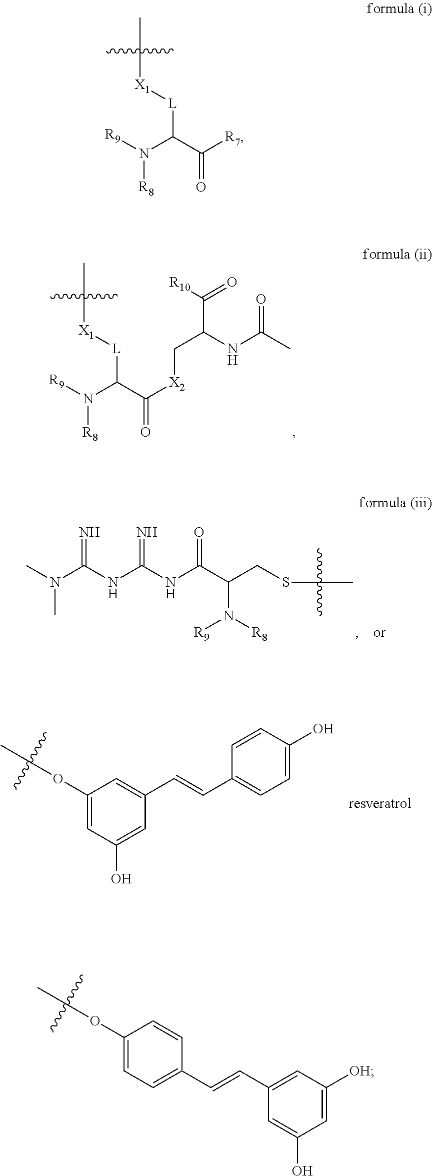

R₇ is (C₁-C₆)alkoxy, (C₁-C₆)alkyl, (C₁-C₆)alkylthio, hydroxy, or —NZ₉Z₁₀;

R₈ is hydrogen or (C₁-C₆)alkyl;

R₉ is hydrogen, (C₁-C₆)alkyl, or (C₁-C₆)alkylcarbonyl;

R₁₀ is (C₁-C₆)alkoxy, (C₁-C₆)alkylthio, hydroxy, or —NZ₉Z₁₀;

X₁ and X₂ are independently O or S;

L is (C₁-C₆)alkylene; and

Z₉ and Z₁₀ are independently hydrogen, (C₁-C₆)alkyl, or (C₁-C₆)alkylcarbonyl.

Representative compounds of Formula (V) include, but are not limited to, the compounds shown below.

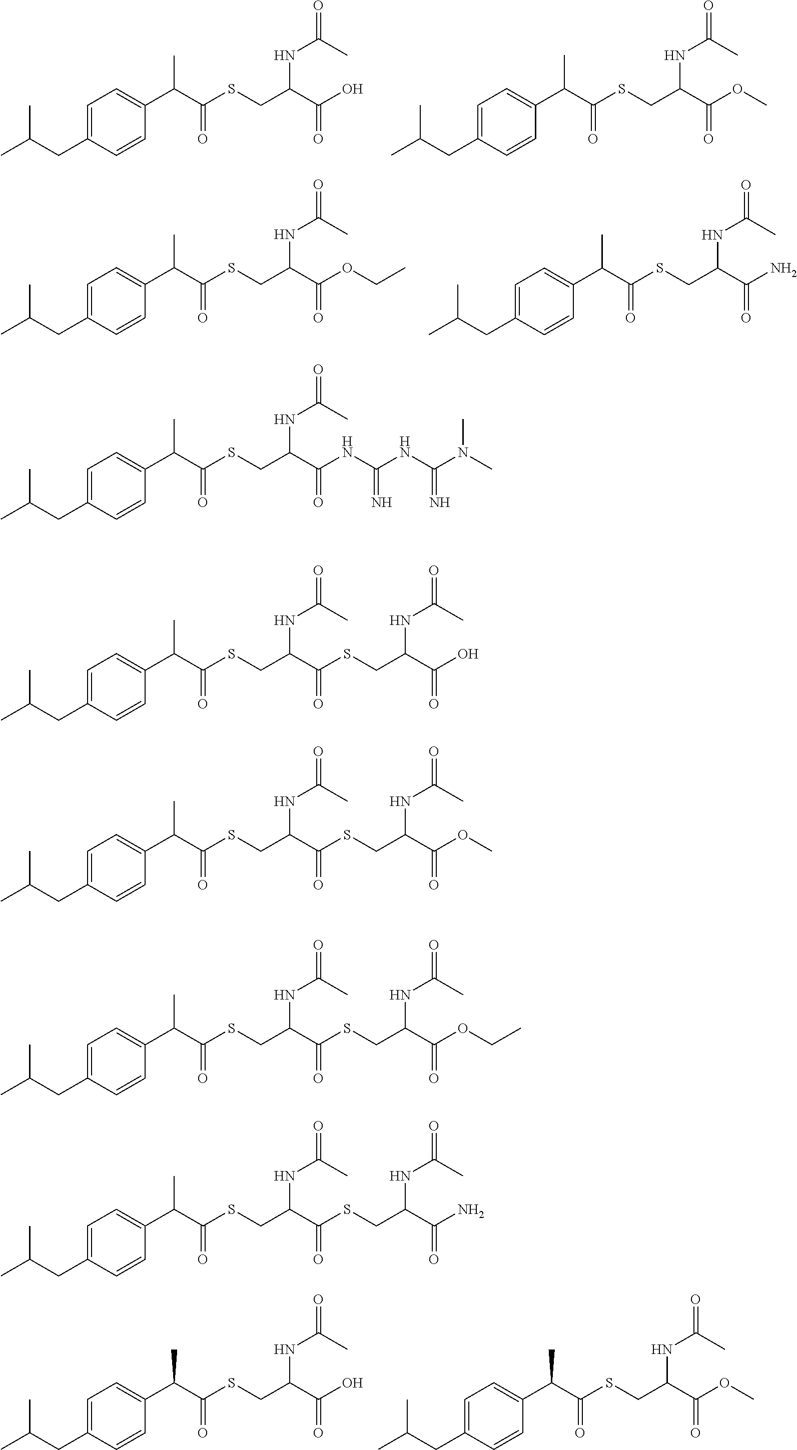

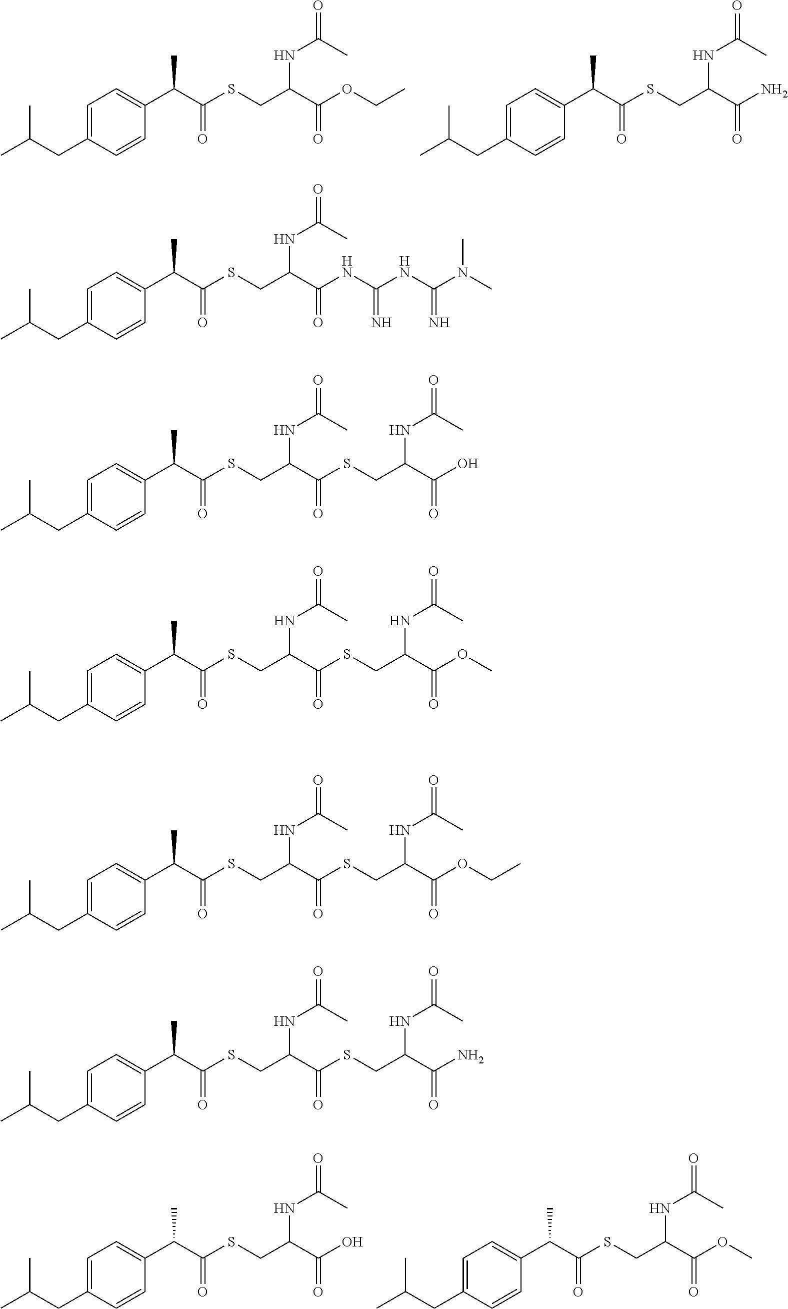

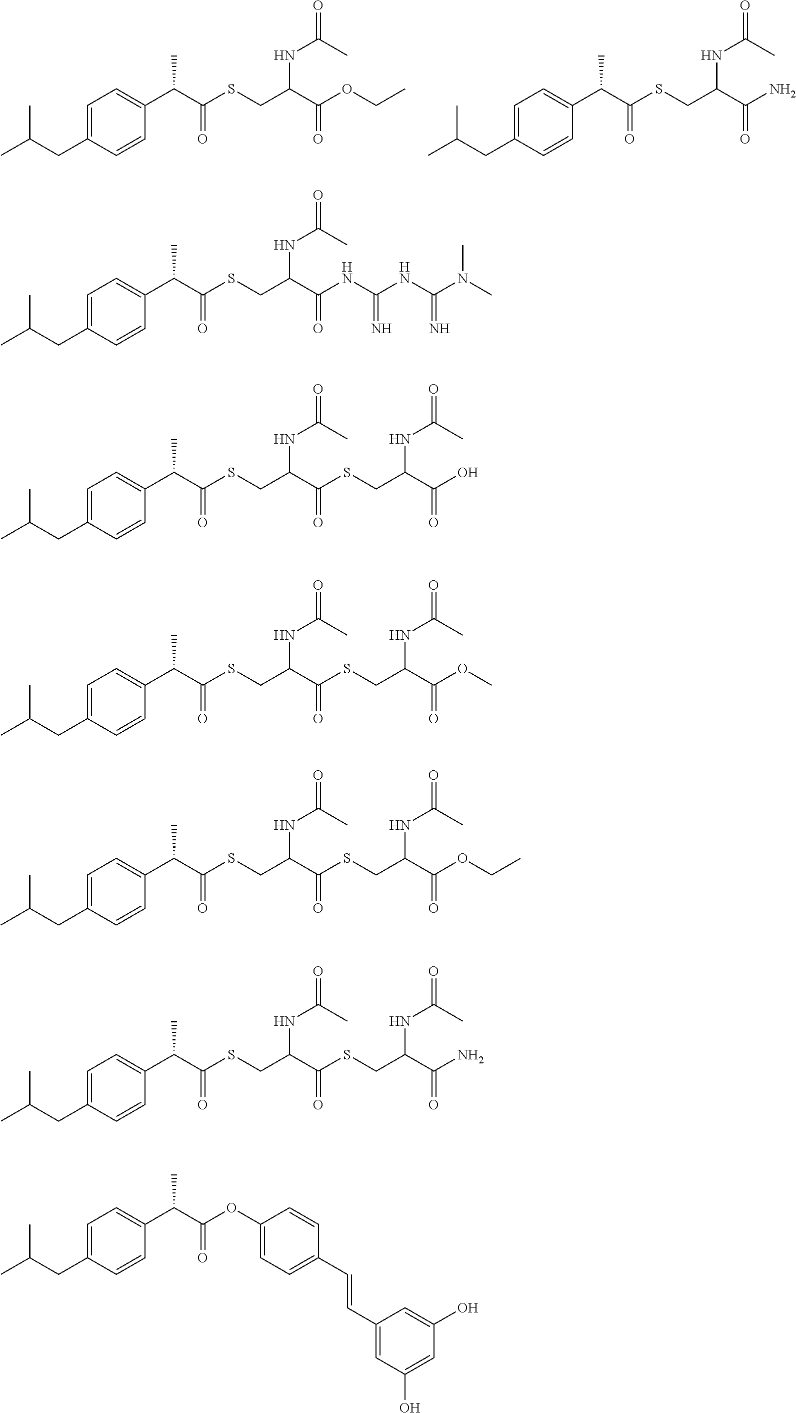

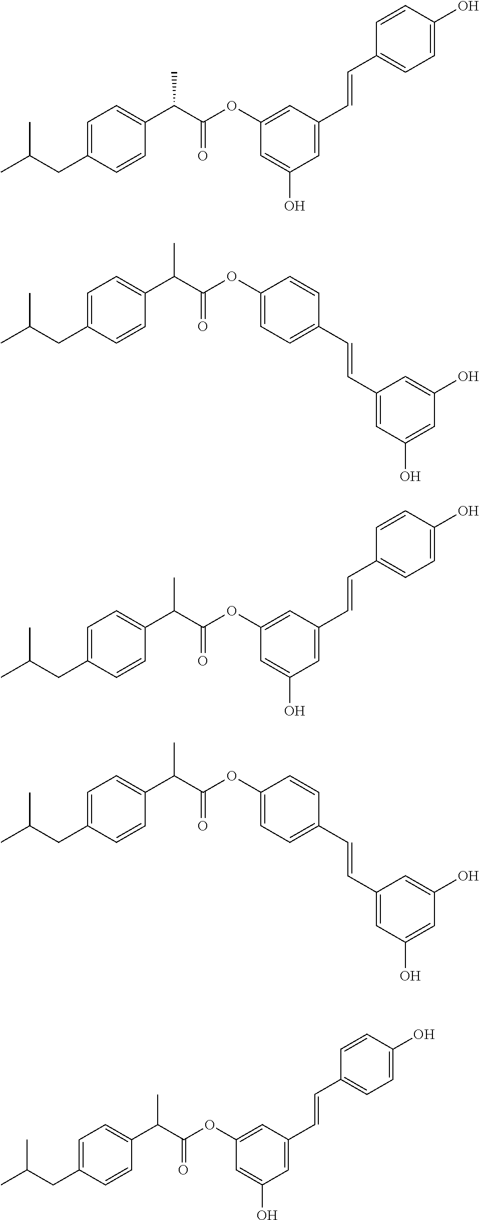

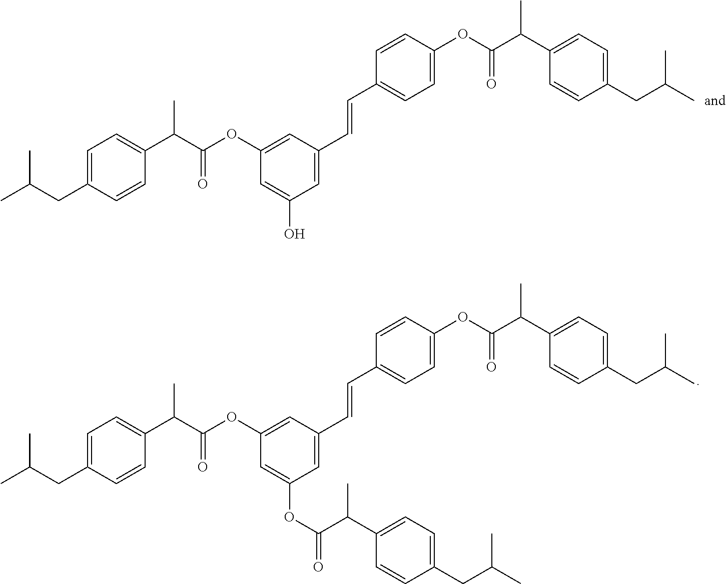

In another aspect, the present invention provides methods for treating atherosclerosis, neuropathy, nephropathy, retinopathy, inflammatory disorders, cardiovascular diseases, and metabolic disorders in a mammal or patient comprising administering to the mammal or patient in need of such treatment a therapeutically effective amount of a compound of Formula (V), as shown above, or a pharmaceutically acceptable salt thereof.

In certain embodiments, the inventive methods include treating dyslipidemia, insulin resistance, β-cell dysfunction, hyperglycemia, metabolic syndrome, and any form of diabetes mellitus including type I and type II diabetes, in a patient comprising administering to the patient in need of such treatment a therapeutically effective amount of a compound of Formula (V), as shown above, or a pharmaceutically acceptable salt thereof.

In certain embodiments, the present invention provides methods for reducing advanced glycated end products and/or lipid peroxidation including, but not limited to, oxidation of low-density lipoproteins in a mammal or patient comprising administering to the mammal or patient in need of such treatment a therapeutically effective amount of a compound of Formula (V), as shown above, or a pharmaceutically acceptable salt thereof.

In certain embodiments, the inventive methods include treating dyslipidemia, insulin resistance, β-cell dysfunction, hyperglycemia, metabolic syndrome, and any form of diabetes mellitus including type I and type II diabetes, in a patient comprising administering to the patient in need of such treatment a therapeutically effective amount of a pharmaceutical composition comprising at least one pharmaceutically acceptable carrier and a compound of Formula (V), as shown above, or a pharmaceutically acceptable salt thereof.

In certain embodiments, the present invention provides methods for reducing advanced glycated end products and lipid peroxidation including, but not limited to, oxidation of low-density lipoproteins, in a patient comprising administering to the patient in need of such treatment a therapeutically effective amount of a pharmaceutical composition comprising at least one pharmaceutically acceptable carrier and a compound of Formula (V), as shown above, or a pharmaceutically acceptable salt thereof.

In another aspect, the present invention provides compounds of Formula (VI)

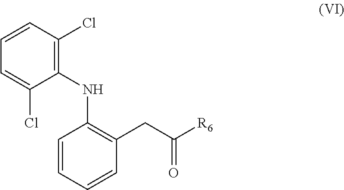

wherein R₆ is

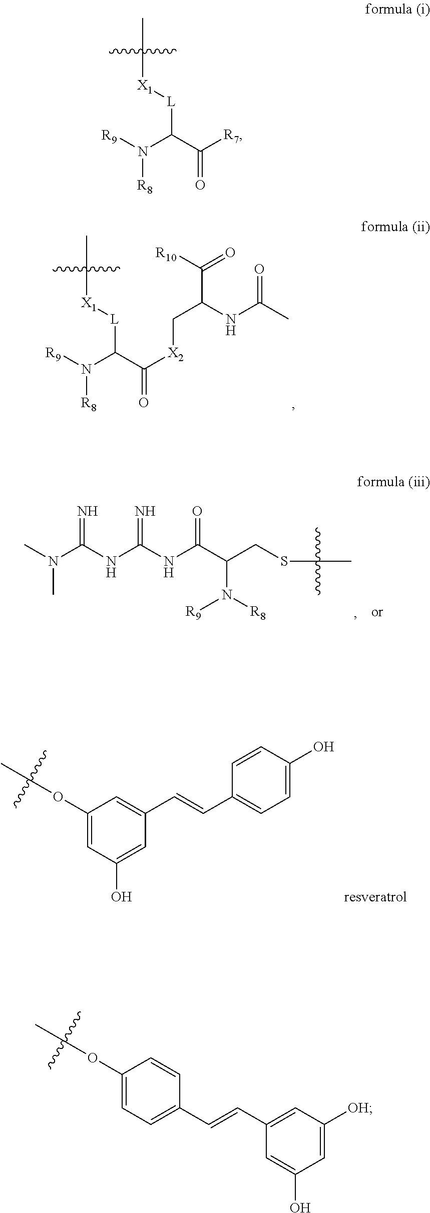

R₇ is (C₁-C₆)alkoxy, (C₁-C₆)alkyl, (C₁-C₆)alkylthio, hydroxy, or —NZ₉Z₁₀;

R₈ is hydrogen or (C₁-C₆)alkyl;

R₉ is hydrogen, (C₁-C₆)alkyl, or (C₁-C₆)alkylcarbonyl;

R₁₀ is (C₁-C₆)alkoxy, (C₁-C₆)alkylthio, hydroxy, or —NZ₉Z₁₀

X₁ and X₂ are independently O or S;

L is (C₁-C₆)alkylene; and

Z₉ and Z₁₀ are independently hydrogen, (C₁-C₆)alkyl, or (C₁-C₆)alkylcarbonyl.

Representative compounds of Formula (VI) include, but are not limited to, the compounds shown below.

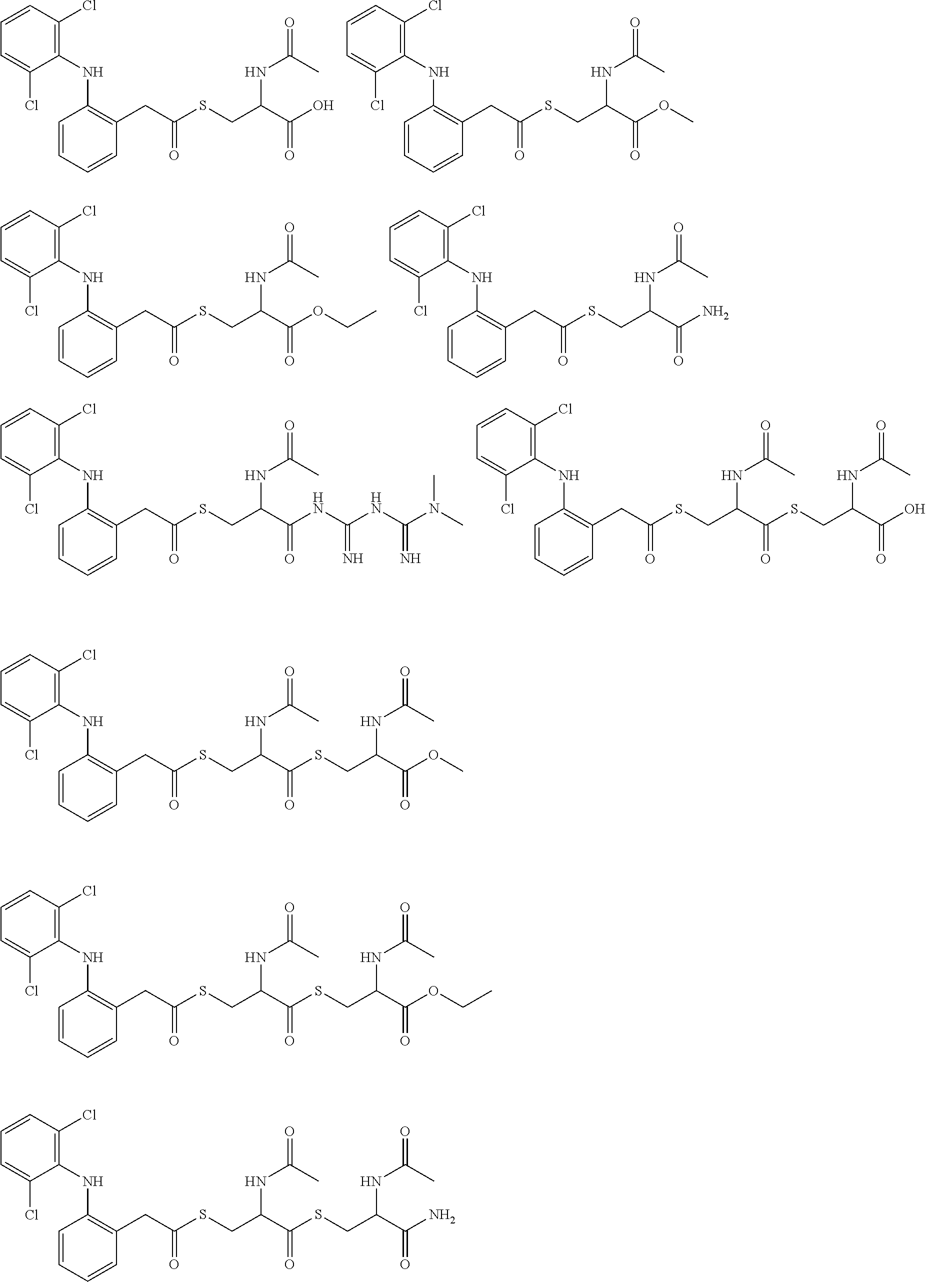

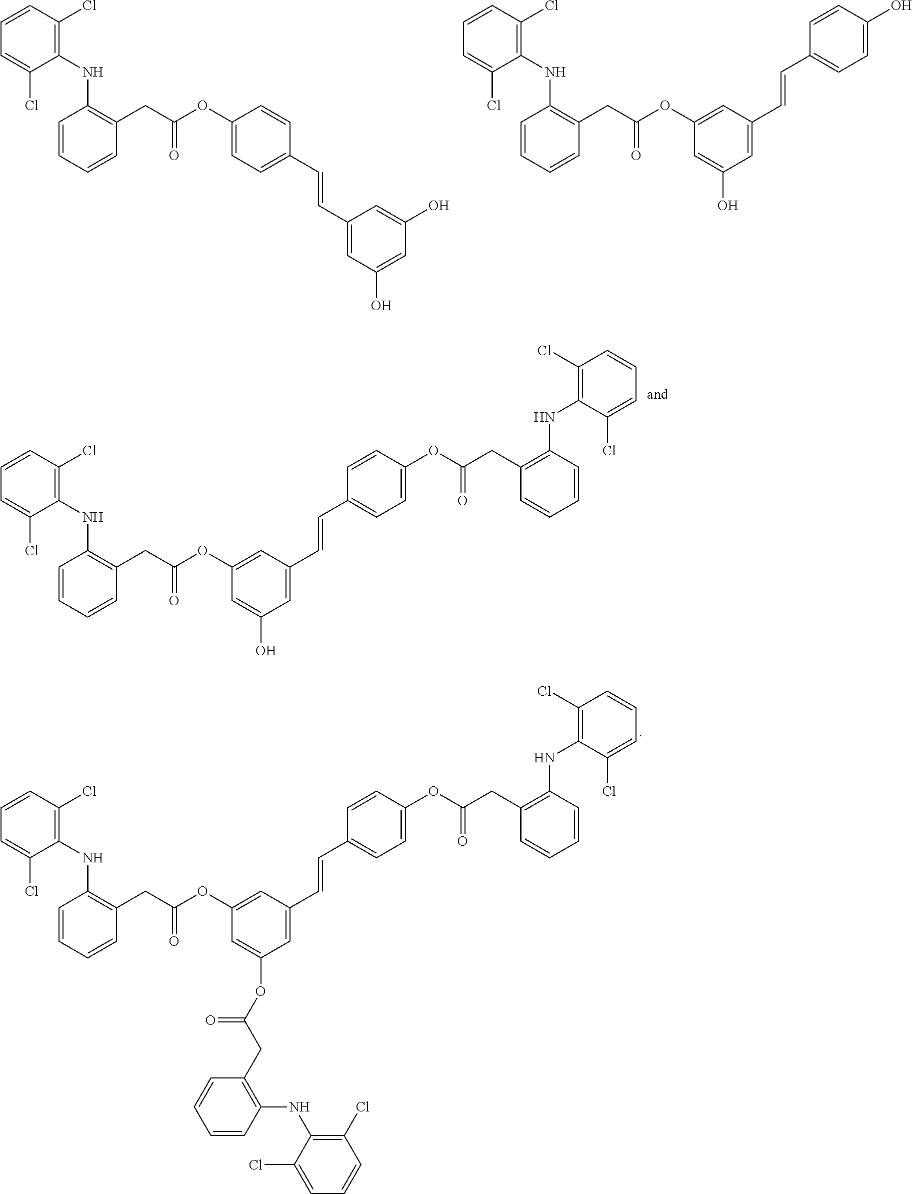 [SMILES: O=C(Cc1ccccc1Nc1c(Cl)cccc1Cl)Oc1ccc(/C=C/c2cc(O)cc(O)c2)cc1 ; O=C(Cc1ccccc1Nc1c(Cl)cccc1Cl)Oc1cc(O)cc(/C=C/c2ccc(O)cc2)c1 ; O=C(Cc1ccccc1Nc1c(Cl)cccc1Cl)Oc1ccc(/C=C/c2cc(O)cc(OC(=O)Cc3ccccc3Nc3c(Cl)cccc3Cl)c2)cc1 ; O=C(Cc1ccccc1Nc1c(Cl)cccc1Cl)Oc1ccc(/C=C/c2cc(OC(=O)Cc3ccccc3Nc3c(Cl)cccc3Cl)cc(OC(=O)Cc3ccccc3Nc3c(Cl)cccc3Cl)c2)cc1]

In another aspect, the present invention provides methods for treating atherosclerosis, neuropathy, nephropathy, retinopathy, inflammatory disorders, cardiovascular diseases, and metabolic disorders in a mammal or patient comprising administering to the mammal or patient in need of such treatment a therapeutically effective amount of a compound of Formula (VI), as shown above, or a pharmaceutically acceptable salt thereof.

In certain embodiments, the inventive methods include treating dyslipidemia, insulin resistance, β-cell dysfunction, hyperglycemia, metabolic syndrome, and any form of diabetes mellitus including type I and type II diabetes, in a patient comprising administering to the patient in need of such treatment a therapeutically effective amount of a compound of Formula (VI), as shown above, or a pharmaceutically acceptable salt thereof.

In certain embodiments, the present invention provides methods for reducing advanced glycated end products and/or lipid peroxidation including, but not limited to, oxidation of low-density lipoproteins in a mammal or patient comprising administering to the mammal or patient in need of such treatment a therapeutically effective amount of a compound of Formula (VI), as shown above, or a pharmaceutically acceptable salt thereof.

In certain embodiments, the inventive methods include treating dyslipidemia, insulin resistance, β-cell dysfunction, hyperglycemia, metabolic syndrome, and any form of diabetes mellitus including type I and type II diabetes, in a patient comprising administering to the patient in need of such treatment a therapeutically effective amount of a pharmaceutical composition comprising at least one pharmaceutically acceptable carrier and a compound of Formula (VI), or a pharmaceutically acceptable salt thereof.

In certain embodiments, the present invention provides methods for reducing advanced glycated end products and lipid peroxidation including, but not limited to, oxidation of low-density lipoproteins, in a patient comprising administering to the patient in need of such treatment a therapeutically effective amount of a pharmaceutical composition comprising at least one pharmaceutically acceptable carrier and a compound of Formula (VI), as shown above, or a pharmaceutically acceptable salt thereof.

In another aspect, the present invention provides compounds of Formula (VII)

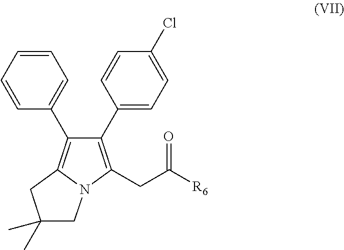

wherein R₆ is

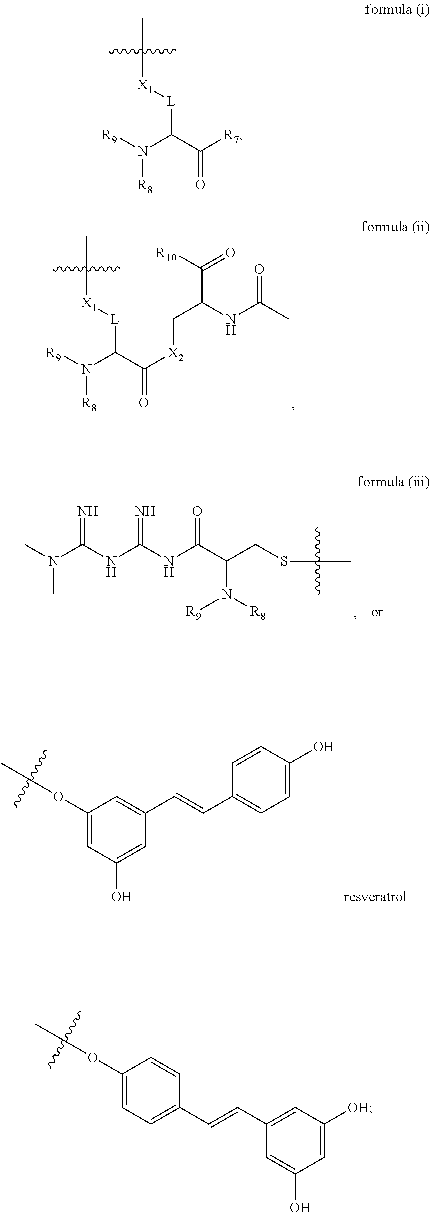

R₇ is (C₁-C₆)alkoxy, (C₁-C₆)alkyl, (C₁-C₆)alkylthio, hydroxy, or —NZ₉Z₁₀;

R₈ is hydrogen or (C₁-C₆)alkyl;

R₉ is hydrogen, (C₁-C₆)alkyl, or (C₁-C₆)alkylcarbonyl;

R₁₀ is (C₁-C₆)alkoxy, (C₁-C₆)alkylthio, hydroxy, or —NZ₉Z₁₀;

X₁ and X₂ are independently O or S;

L is (C₁-C₆)alkylene; and

Z₉ and Z₁₀ are independently hydrogen, (C₁-C₆)alkyl, or (C₁-C₆)alkylcarbonyl.

Representative compounds of Formula (VII) include, but are not limited to, the compounds shown below.

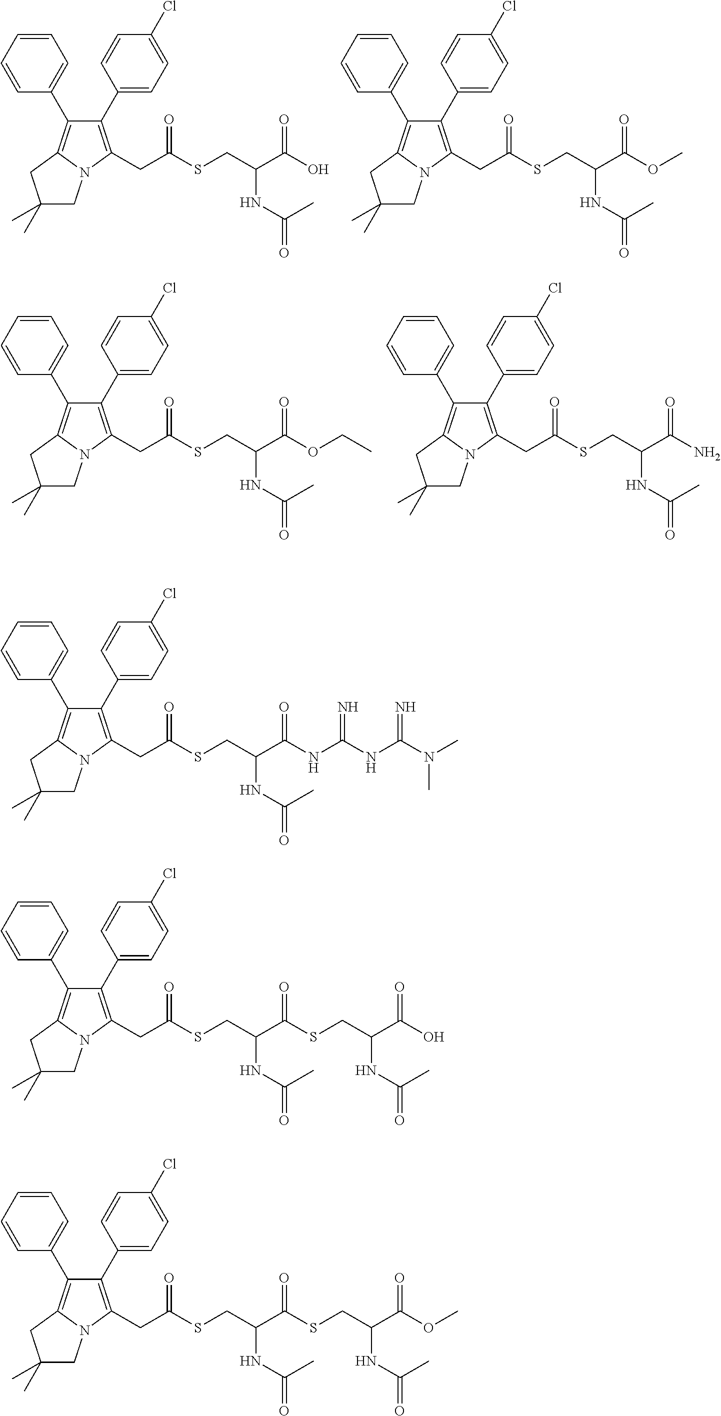

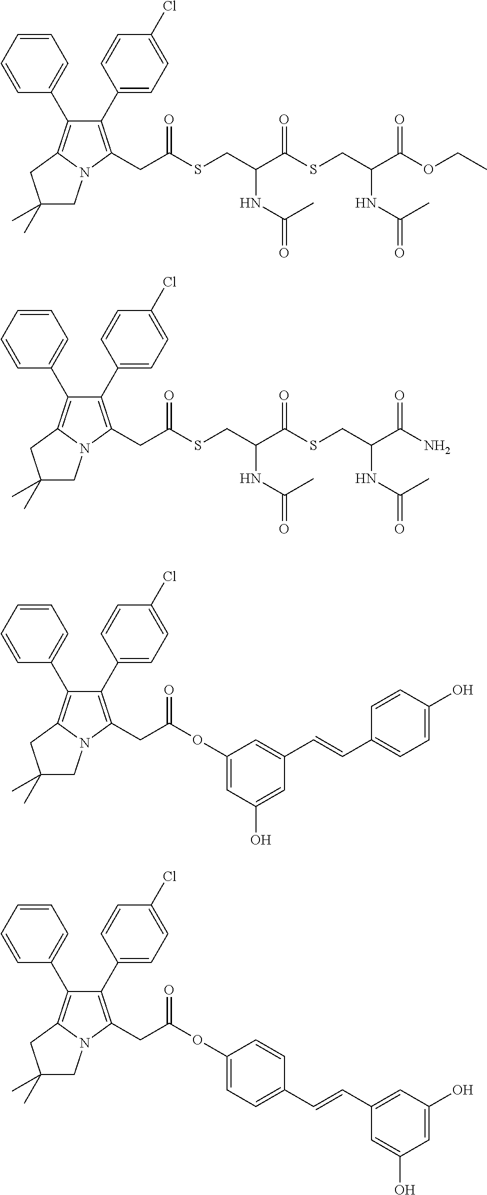 [SMILES: CCOC(=O)C(CSC(=O)C(CSC(=O)Cc1c(-c2ccc(Cl)cc2)c(-c2ccccc2)c2n1CC(C)(C)C2)NC(C)=O)NC(C)=O ; CC(=O)NC(CSC(=O)C(CSC(=O)Cc1c(-c2ccc(Cl)cc2)c(-c2ccccc2)c2n1CC(C)(C)C2)NC(C)=O)C(N)=O ; CC1(C)Cc2c(-c3ccccc3)c(-c3ccc(Cl)cc3)c(CC(=O)Oc3cc(O)cc(/C=C/c4ccc(O)cc4)c3)n2C1 ; CC1(C)Cc2c(-c3ccccc3)c(-c3ccc(Cl)cc3)c(CC(=O)Oc3ccc(/C=C/c4cc(O)cc(O)c4)cc3)n2C1]

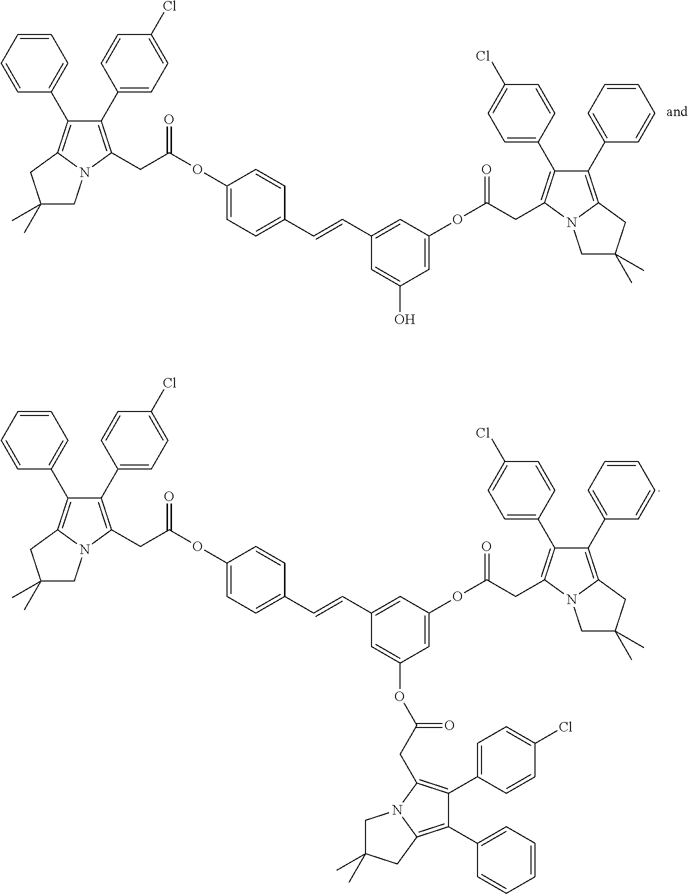 [SMILES: CC1(C)Cc2c(-c3ccccc3)c(-c3ccc(Cl)cc3)c(CC(=O)Oc3ccc(/C=C/c4cc(O)cc(OC(=O)Cc5c(-c6ccc(Cl)cc6)c(-c6ccccc6)c6n5CC(C)(C)C6)c4)cc3)n2C1 ; CC1(C)Cc2c(-c3ccccc3)c(-c3ccc(Cl)cc3)c(CC(=O)Oc3ccc(/C=C/c4cc(OC(=O)Cc5c(-c6ccc(Cl)cc6)c(-c6ccccc6)c6n5CC(C)(C)C6)cc(OC(=O)Cc5c(-c6ccc(Cl)cc6)c(-c6ccccc6)c6n5CC(C)(C)C6)c4)cc3)n2C1]

In another aspect, the present invention provides methods for treating atherosclerosis, neuropathy, nephropathy, retinopathy, inflammatory disorders, cardiovascular diseases, and metabolic disorders in a mammal or patient comprising administering to the mammal or patient in need of such treatment a therapeutically effective amount of a compound of Formula (VII), as shown above, or a pharmaceutically acceptable salt thereof.

In certain embodiments, the inventive methods include treating dyslipidemia, insulin resistance, β-cell dysfunction, hyperglycemia, metabolic syndrome, and any form of diabetes mellitus including type I and type II diabetes, in a patient comprising administering to the patient in need of such treatment a therapeutically effective amount of a compound of Formula (VII), as shown above, or a pharmaceutically acceptable salt thereof.

In certain embodiments, the present invention provides methods for reducing advanced glycated end products and/or lipid peroxidation including, but not limited to, oxidation of low-density lipoproteins in a mammal or patient comprising administering to the mammal or patient in need of such treatment a therapeutically effective amount of a compound of Formula (VI), as shown above, or a pharmaceutically acceptable salt thereof.

In certain embodiments, the inventive methods include treating dyslipidemia, insulin resistance, β-cell dysfunction, hyperglycemia, metabolic syndrome, and any form of diabetes mellitus including type I and type II diabetes, in a patient comprising administering to the patient in need of such treatment a therapeutically effective amount of a pharmaceutical composition comprising at least one pharmaceutically acceptable carrier and a compound of Formula (VII), as shown above, or a pharmaceutically acceptable salt thereof.

In certain embodiments, the present invention provides methods for reducing advanced glycated end products and lipid peroxidation including, but not limited to, oxidation of low-density lipoproteins, in a patient comprising administering to the patient in need of such treatment a therapeutically effective amount of a pharmaceutical composition comprising at least one pharmaceutically acceptable carrier and a compound of Formula (VII), as shown above, or a pharmaceutically acceptable salt thereof.

In another aspect, the present invention provides methods for treating adipocyte dysfunction related diseases, carbohydrate metabolism related diseases, vascular diseases, neurodegenerative diseases, cancers, arthritis, osteoarthritis, spondylitis, bone resorption diseases, sepsis, septic shock, chronic pulmonary inflammatory disease, fever, periodontal diseases, ulcerative colitis, pyresis, Alzheimer's disease, Parkinson's diseases, cystic fibrosis, dysfunctions of the immune system, stroke, multiple sclerosis, migraine, pain, inflammatory eye conditions including uveitis, glaucoma and conjunctivitis, degenerative bone or joint conditions including osteoarthritis, rheumatoid arthritis, rheumatoid spondylitis, gouty arthritis ankylosing spondylitis, psoriatic arthritis and other arthritic conditions, as well as inflamed joints, chronic inflammatory skin conditions, including allergic lesions, lichen planus, pityriasis rosea, eczema, psoriasis, and dermatitis, diseases and disorders of the gastrointestinal tract, including inflammatory bowel disease, Crohn's disease, atrophic gastritis, gastritis varialoforme, ulcerative colitis, coeliac disease, regional ileitis, peptic ulceration, particularly irritable bowel syndrome, reflux oesophagitis, and damage to the gastrointestinal tract resulting from infections, for example, by Helicobacter inflammatory lung disorders such as asthma, bronchitis, particularly chronic obstructive pulmonary disease, farmer's lung, acute respiratory distress syndrome; bacteraemia, endotoxaemia (septic shock), aphthous ulcers, gingivitis, pyresis, particularly pain, including inflammatory pain, neuropathic pain, acute pain or pain of a central origin; meningitis and pancreatitis, and other conditions associated with inflammation, central nervous system inflammatory conditions and diseases, including ischaemia-reperfusion injury associated with ischemic stroke; vascular diseases, such as atheromatous and nonatheromatous, ischemic heart disease, and Raynaud's Disease and Phenomenon in a mammal or patient comprising administering to the mammal or patient in need of such treatment a therapeutically effective amount of a compound of Formulae (I-VII), or a pharmaceutically acceptable salt thereof. In certain embodiments, the present invention provides uses for compounds of Formula (I-VII) for preparing, or for the manufacture of, a medicament for treating the diseases/disorders listed above.

In another aspect, the present invention provides methods for treating adipocyte dysfunction related diseases, carbohydrate metabolism related diseases, vascular diseases, neurodegenerative diseases, cancers, arthritis, osteoarthritis, spondylitis, bone resorption diseases, sepsis, septic shock, chronic pulmonary inflammatory disease, fever, periodontal diseases, ulcerative colitis, pyresis, Alzheimer's disease, Parkinson's diseases, cystic fibrosis, dysfunctions of the immune system, stroke, multiple sclerosis, migraine, pain, inflammatory eye conditions including uveitis, glaucoma and conjunctivitis, degenerative bone or joint conditions including osteoarthritis, rheumatoid arthritis, rheumatoid spondylitis, gouty arthritis ankylosing spondylitis, psoriatic arthritis and other arthritic conditions, as well as inflamed joints, chronic inflammatory skin conditions, including allergic lesions, lichen planus, pityriasis rosea, eczema, psoriasis, and dermatitis, diseases and disorders of the gastrointestinal tract, including inflammatory bowel disease, Crohn's disease, atrophic gastritis, gastritis varialoforme, ulcerative colitis, coeliac disease, regional ileitis, peptic ulceration, particularly irritable bowel syndrome, reflux oesophagitis, and damage to the gastrointestinal tract resulting from infections, for example, by Helicobacter inflammatory lung disorders such as asthma, bronchitis, particularly chronic obstructive pulmonary disease, farmer's lung, acute respiratory distress syndrome; bacteraemia, endotoxaemia (septic shock), aphthous ulcers, gingivitis, pyresis, particularly pain, including inflammatory pain, neuropathic pain, acute pain or pain of a central origin; meningitis and pancreatitis, and other conditions associated with inflammation, central nervous system inflammatory conditions and diseases, including ischaemia-reperfusion injury associated with ischemic stroke; vascular diseases, such as atheromatous and nonatheromatous, ischemic heart disease, and Raynaud's Disease and Phenomenon in a patient comprising administering to the patient in need of such treatment a therapeutically effective amount of a pharmaceutical composition comprising at least one pharmaceutically acceptable carrier and a compound of Formula (I-VII), or a pharmaceutically acceptable salt thereof. In certain embodiments, the present invention provides uses for pharmaceutical compositions for preparing, or for the manufacture of, a medicament for treating the diseases/disorders listed above, wherein the pharmaceutical composition comprises at least one pharmaceutically acceptable carrier and a compound of Formula (I-VII), or a pharmaceutically acceptable salt thereof,

### DEFINITIONS

As used throughout this specification and the appended claims, the following terms have the following meanings:

The term “(C₁-C₆)alkoxy” as used herein, means a (C₁-C₆)alkyl group, as defined herein, appended to the parent molecular moiety through an oxygen atom. Representative examples of (C₁-C₆)alkoxy include, but are not limited to, methoxy, ethoxy, propoxy, 2-propoxy, butoxy, tert-butoxy, pentyloxy, and hexyloxy.

The term “(C₁-C₆)alkoxycarbonyl” as used herein, means a (C₁-C₆)alkoxy group, as defined herein, appended to the parent molecular moiety through a carbonyl group, as defined herein. Representative examples of (C₁-C₆)alkoxycarbonyl include, but are not limited to, methoxycarbonyl, ethoxycarbonyl, and tert-butoxycarbonyl.

The term “(C₁-C₆)alkoxysulfonyl” as used herein, means a (C₁-C₆)alkoxy group, as defined herein, appended to the parent molecular moiety through a sulfonyl group, as defined herein. Representative examples of (C₁-C₆)alkoxysulfonyl include, but are not limited to, methoxysulfonyl, ethoxysulfonyl and propoxysulfonyl.

The term “(C₁-C₆)alkyl” as used herein, means a straight or branched chain hydrocarbon containing from 1 to 8 carbon atoms. Representative examples of (C₁-C₆)alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, and hexyl.

The term “(C₁-C₆)alkylcarbonyl” as used herein, means a (C₁-C₆)alkyl group, as defined herein, appended to the parent molecular moiety through a carbonyl group, as defined herein. Representative examples of (C₁-C₆)alkylcarbonyl include, but are not limited to, acetyl, 1-oxopropyl, 2,2-dimethyl-1-oxopropyl, 1-oxobutyl, and 1-oxopentyl.

The term “(C₁-C₆)alkylcarbonyloxy” as used herein, means a (C₁-C₆)alkylcarbonyl group, as defined herein, appended to the parent molecular moiety through an oxygen atom. Representative examples of (C₁-C₆)alkylcarbonyloxy include, but are not limited to, acetyloxy, ethylcarbonyloxy, and tert-butylcarbonyloxy.

The term “(C₁-C₆)alkylene” means a divalent group derived from a straight or branched chain hydrocarbon of from 1 to 6 carbon atoms. Representative examples of (C₁-C₆)alkylene include, but are not limited to, —CH₂—, —CH(CH₃)—, —C(CH₃)₂—, —CH₂CH₂—, —CH₂CH₂CH₂—, —CH₂CH₂CH₂CH₂—, —CH₂CH(CH₃)CH₂—, and —CH₂CH₂CH₂CH₂CH₂CH₂—.

The term “(C₁-C₆)alkylsulfonyl” as used herein, means an (C₁-C₆)alkyl group, as defined herein, appended to the parent molecular moiety through a sulfonyl group, as defined herein. Representative examples of (C₁-C₆)alkylsulfonyl include, but are not limited to, methylsulfonyl and ethylsulfonyl.

The term “(C₁-C₆)alkylthio” as used herein, means a (C₁-C₆)alkyl group, as defined herein, appended to the parent molecular moiety through a sulfur atom. Representative examples of (C₁-C₆)alkylthio include, but are not limited, methylthio, ethylthio, tert-butylthio, and hexylthio.

The term “carbonyl” as used herein, means a —C(O)— group.

The term “carboxy” as used herein, means a —CO₂H group.

The term “cyano” as used herein, means a —CN group.

The term “formyl” as used herein, means a —C(O)H group.

The term “halo” or “halogen” as used herein, means —Cl, —Br, —I or —F.

The term “halo(C₁-C₆)alkoxy” as used herein, means at least one halogen, as defined herein, appended to the parent molecular moiety through a (C₁-C₆)alkoxy group, as defined herein. Representative examples of halo(C₁-C₆)alkoxy include, but are not limited to, chloromethoxy, 2-fluoroethoxy, trifluoromethoxy, and pentafluoroethoxy.

The term “halo(C₁-C₆)alkyl” as used herein, means at least one halogen, as defined herein, appended to the parent molecular moiety through a (C₁-C₆)alkyl group, as defined herein. Representative examples of halo(C₁-C₆)alkyl include, but are not limited to, chloromethyl, 2-fluoroethyl, trifluoromethyl, pentafluoroethyl, and 2-chloro-3-fluoropentyl.

The term “HTB” as used herein means 2-hydroxy-4-(trifluoromethyl)benzoic acid, a metabolite of triflusal. Conjugates comprised of HTB and one or more antioxidants are specifically contemplated by the present invention.

The term “hydroxy” as used herein, means an —OH group.

The term “hydroxy(C₁-C₆)alkyl” as used herein, means at least one hydroxy group, as defined herein, is appended to the parent molecular moiety through a (C₁-C₆)alkyl group, as defined herein. Representative examples of hydroxy(C₁-C₆)alkyl include, but are not limited to, hydroxymethyl, 2-hydroxyethyl, 3-hydroxypropyl, and 2,3-dihydroxypentyl.

The term “mercapto” as used herein, means a —SH group.

The term “nitro” as used herein, means a —NO₂ group.

The term “sulfonyl” as used herein, means a —SO₂— group.

Compounds of the present invention include α-amino acids, or derivatives thereof such as esters or amides, that can exist as stereoisomers, wherein the asymmetric or chiral center is present at the α-carbon. The chiral center is designated (L) or (D) based on the Fischer projections of (L) or (D) aldose. Ernest L. Eliel and Samuel H. Wilen, Stereochemistry of Organic Compounds, John Wiley & Sons, Inc., New York, page 112, 1994. Further, compounds of the present invention may contain a stereocenter that is not an α-carbon of an α-amino acid (or derivative thereof). This center is designated (R) or (S), depending on the configuration of substituents around the chiral carbon atom. The terms (R) and (S) used herein are configurations as defined in IUPAC 1974 Recommendations for Section E, Fundamental Stereochemistry, Pure Appl. Chem., (1976), 45: 13-30. Individual stereoisomers of compounds of the present invention may be prepared synthetically from commercially available starting materials which contain asymmetric or chiral centers or by preparation of racemic mixtures followed by resolution, a technique well-known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) direct separation of the mixture of optical enantiomers on chiral chromatographic columns, or (3) formation of a diastereomeric salt followed by selective recrystallization of one of the diastereomeric salts.

The present invention also provides pharmaceutical compositions which comprise compounds of the present invention formulated together with one or more non-toxic pharmaceutically acceptable carriers. The pharmaceutical compositions may be specially formulated for oral administration in solid or liquid form, for parenteral injection, or for rectal administration.

The term “pharmaceutically acceptable carrier” as used herein means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The present invention provides pharmaceutical compositions which comprise compounds of the present invention formulated together with one or more non-toxic pharmaceutically acceptable carriers. The pharmaceutical compositions can be formulated for oral administration in solid or liquid form, for parenteral injection or for rectal administration.

The pharmaceutical compositions of this invention can be administered to humans (patients) and other mammals orally, rectally, parenterally, intracisternally, intraperitoneally, topically (as by powders, ointments or drops), bucally or as an oral or nasal spray. The term “parenterally,” as used herein, refers to modes of administration which include intravenous, intramuscular, intraperitoneal, intrasternal, subcutaneous, intraarticular injection and infusion.

Pharmaceutical compositions of this invention for parenteral injection comprise pharmaceutically acceptable sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity may be maintained, for example, by the use of a coating such as lecithin, by the maintenance of the required particle size in the case of dispersions, and by the use of surfactants.

These compositions may also contain adjuvants such as preservative agents, wetting agents, emulsifying agents, and dispersing agents. Prevention of the action of microorganisms may be ensured by various antibacterial and antifungal agents, for example, parabens, chlorobutanol, phenol, sorbic acid, and the like. It may also be desirable to include isotonic agents, for example, sugars, sodium chloride and the like. Prolonged absorption of the injectable pharmaceutical form may be brought about by the use of agents delaying absorption, for example, aluminum monostearate and gelatin.

In some cases, in order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Suspensions, in addition to the active compounds, may contain suspending agents, as, for example, ethoxylated isostearyl alcohols, polyoxyethylene sorbitol and sorbitan esters, microcrystalline cellulose, aluminum metahydroxide, bentonite, agar-agar, tragacanth, and mixtures thereof.

If desired, and for more effective distribution, the compounds of the present invention can be incorporated into slow-release or targeted-delivery systems such as polymer matrices, liposomes, and microspheres. They may be sterilized, for example, by filtration through a bacteria-retaining filter or by incorporation of sterilizing agents in the form of sterile solid compositions, which may be dissolved in sterile water or some other sterile injectable medium immediately before use.

The active compounds can also be in micro-encapsulated form, if appropriate, with one or more pharmaceutically acceptable carriers as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound can be admixed with at least one inert diluent such as sucrose, lactose, or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of such composition that they release the active ingredient(s) only, or preferentially, in a certain part of the intestinal tract in a delayed manner. Examples of embedding compositions which can be used include polymeric substances and waxes.

Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides) Depot injectable formulations are also prepared by entrapping the drug in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic, parenterally acceptable diluent or solvent such as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Solid dosage forms for oral administration include capsules, tablets, pills, powders, and granules. In such solid dosage forms, the active compound is mixed with at least one inert pharmaceutically acceptable carrier such as sodium citrate or calcium phosphate and/or a) fillers or extenders such as starches, lactose, sucrose, glucose, mannitol, and salicylic acid; b) binders such as carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay; and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract in a delayed manner. Examples of embedding compositions which can be used include polymeric substances and waxes.

Compositions for rectal administration are preferably suppositories which can be prepared by mixing the compounds of this invention with suitable non-irritating carriers such as cocoa butter, polyethylene glycol or a suppository wax which are solid at ambient temperature but liquid at body temperature and therefore melt in the rectum and release the active compound.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, and perfuming agents.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this invention.

The ointments, pastes, creams and gels may contain, in addition to an active compound of this invention, animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, lactose, talc, silicic acid, aluminum hydroxide, calcium silicates and polyamide powder, or mixtures of these substances. Sprays can additionally contain customary propellants such as chlorofluorohydrocarbons.

Compounds of the present invention may also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes may be used. The present compositions in liposome form may contain, in addition to the compounds of the present invention, stabilizers, preservatives, and the like. The preferred lipids are the natural and synthetic phospholipids and phosphatidylcholines (lecithins) used separately or together.

Methods to form liposomes are known in the art. See, for example, Prescott, Ed., Methods in Cell Biology, Volume XIV, Academic Press, New York, N.Y., (1976), p 33 et seq.

The phrase “therapeutically effective amount” of the compound of the present invention means a sufficient amount of the compound to treat metabolic disorders, at a reasonable benefit/risk ratio applicable to any medical treatment. The specific therapeutically effective dose level for any particular patient will depend upon a variety of factors including the disorder being treated and the severity of the disorder; activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

Actual dosage levels of active ingredients in the pharmaceutical compositions of this invention can be varied so as to obtain an amount of the active compound(s) which is effective to achieve the desired therapeutic response for a particular patient, compositions, and mode of administration. The selected dosage level will depend upon the activity of the particular compound, the route of administration, the severity of the condition being treated, and the condition and prior medical history of the patient being treated.

The total daily dose of the compounds of this invention administered to a mammal, and particularly a human, from about 0.03 to about 20 mg/kg/day. For purposes of oral administration, more preferable doses can be in the range of from about 0.1 to about 10 mg/kg/day. If desired, the effective daily dose can be divided into multiple doses for purposes of administration, e.g. two to four separate doses per day.

The term “pharmaceutically acceptable salt,” as used herein, means a positively-charged inorganic or organic cation that is generally considered suitable for human consumption. Examples of pharmaceutically acceptable cations are alkali metals (lithium, sodium and potassium), magnesium, calcium, ferrous, ferric, ammonium, alkylammonium, dialkylammonium, trialkylammonium, tetraalkylammonium, diethanolammonium, and choline. Cations may be interchanged by methods known in the art, such as ion exchange. Where compounds of the present invention are prepared in the carboxylic acid form, addition of a base (such as a hydroxide or a free amine) will yield the appropriate salt form.

The present invention contemplates pharmaceutically active metabolites formed by in vivo biotransformation of compounds of Formula (I). The term pharmaceutically active metabolite, as used herein, means a compound formed by the in vivo biotransformation of compounds of Formula (I). The present invention contemplates compounds of Formula (I) and metabolites thereof. A thorough discussion of biotransformation is provided in (Goodman and Gilman's, The Pharmacological Basis of Therapeutics, seventh edition).

All patents, patent applications, and literature references cited in the specification are herein incorporated by reference in their entirety for any purpose.

The following Schemes and Examples are provided for the purposes of illustration and are not intended to limit the scope of the present invention. The invention is not limited in scope by the exemplified embodiments, which are intended as illustrations of individual aspects of the invention. Various modifications of the invention in addition to those shown and described herein will become apparent to those skilled in the art from the foregoing description and accompanying drawings. Such modifications are intended to fall within the scope of the appended claims.

### Preparation of Compounds of the Invention

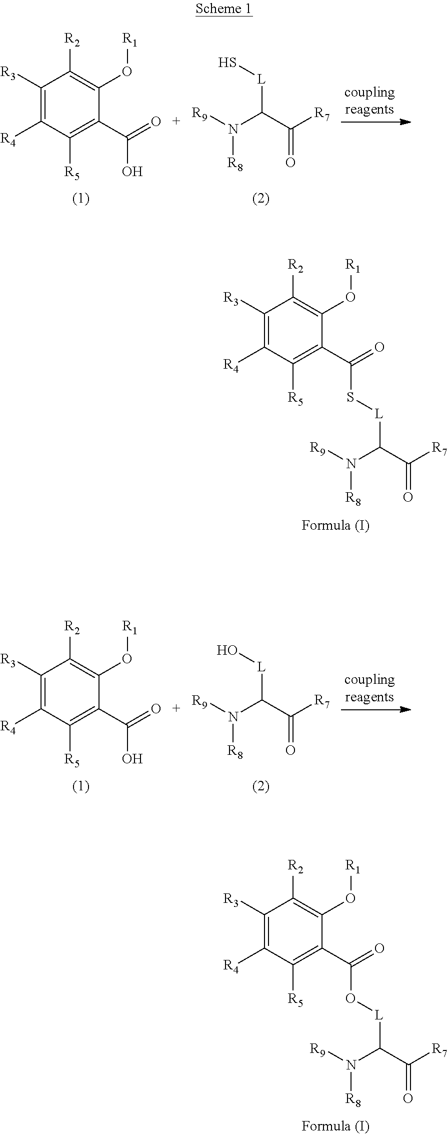

Compounds of Formula (I), wherein R₁, R₂, R₃, R₄, R₅, R₇, R₈, R₉, and L are as defined in the Summary section herein, are prepared as described EP 0 080 229, BE 900328, or Scheme 1. Acids of formula (I) are treated with an alcohol or mercaptan of formula (2) in an appropriate solvent optionally with heating and optionally with one or more coupling reagents to provide compounds of Formula (I). Coupling reagents useful for preparing compounds of the present invention include, but are not limited to, dimethylaminopyridine (DMAP), tert-butylcarbodiimide, 1,1′-carbonyldiimidazole (CDI), 1,1′-thiocarbonyldiimidazole, carbonylbis(2-methylimidazole), 1-(3-dimethylaminopropyl)-3-ethylcarbodiimide hydrochloride (EDCI), benzotriazol-1-yl-oxy-tris-pyrrolidino-phosphoniumhexafluorophosphate (PyBOP), bromo-tris-pyrrolidino-phosphonium hexafluorophosphate (PyBrop), O-(-7-azabenzotriazol-1-yl)-N,N,N′,N′,-tetramethyluronium hexafluorophosphate, N-[(dimethylamino)(3H-[1,2,3]triazolo[4,5-b]pyridin-3-yloxy)methylene]-N-methylmethanaminium, benzotriazol-1-yloxytris(dimethylamino)phosphonium hexafluorophosphate (BOP), Bis(2-oxo-3-oxazolidinyl)phosphinic chloride (BOPCl), 1,3-dicyclohexylcarbodiimide (DCC), 1-Hydroxy-7-azabenzotriazole (HOAT), 1-hydroxybenzotriazole hydrate (HOBT), 3-hydroxy-1,2,3-benzotriazin-4(3H)-one (HOOBT), O-(7-azabenzotriazol-1-yl)-N,N,N′,N tetramethyluronium hexafluorophosphate (HATU), tetramethyluronium hexafluorophosphate (HBTU), and O-benzotriazol-1-yl-N,N,N′,N′-tetramethyluronium tetrafluoroborate (TBTU).

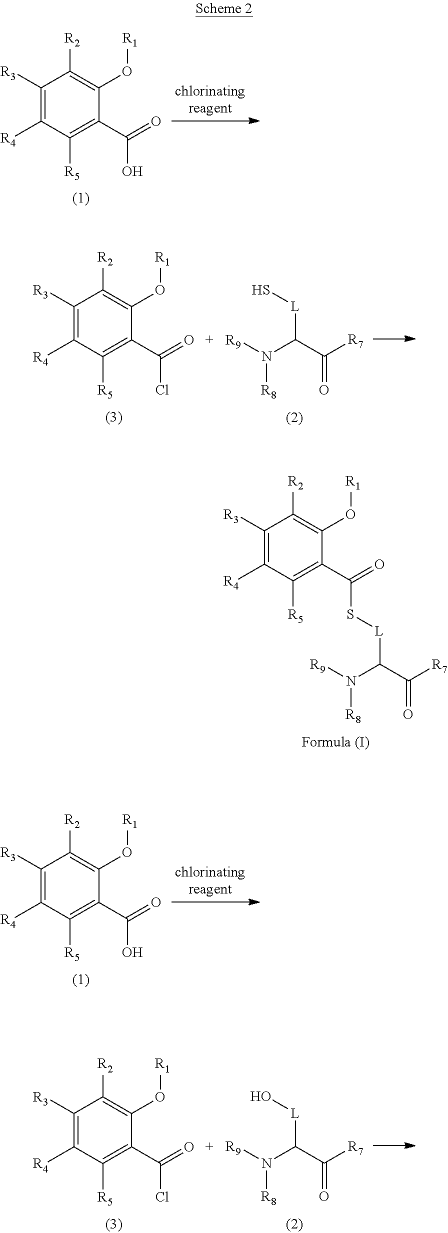

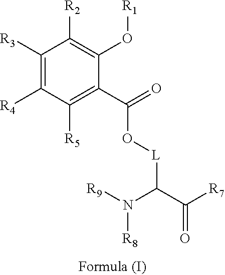

Alternatively, compounds of Formula (I), wherein R₁, R₂, R₃, R₄, R₅, R₇, R₉, and L are as defined in Formula (I) of the Summary section herein, are prepared as described in Scheme 2. Acids of formula (I) are treated with a chlorinating reagent such as thionyl chloride (or PCl₃) in an appropriate solvent to provide acid chlorides of formula (3). Compounds of formula (3) are treated with a base such as triethylamine (or diisopropylethylamine) and an alcohol or thiol of formula (2) in an appropriate solvent, optionally with heating, to provide compounds of Formula (I).

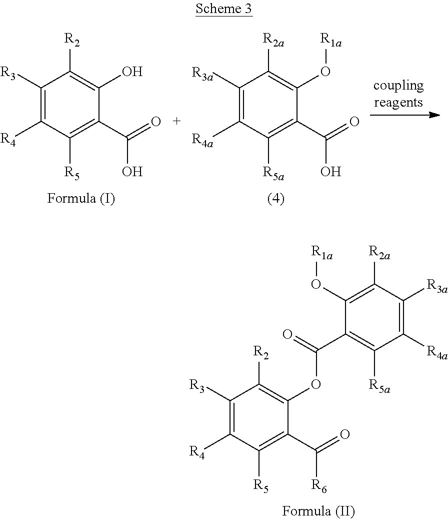

Compounds of Formula (II), wherein R₂, R₃, R₄, R₅, R₆, R₁ₐ, R₂ₐ, R₃ₐ, R₄ₐ, and R₅ₐ, are as defined in Formula (I) of the Summary section herein, are prepared as described in Scheme 3. Compounds of Formula (I) are treated with a benzoic acid of formula (4) in the presence of one or more coupling reagents, as disclosed in Scheme 1, in an appropriate solvent to provide compounds of Formula (II). Alternatively, a compound of formula (4) can be treated with a chlorinating agent (see Scheme 2) and a base including, but not limited to triethylamine or diisopropylethylamine, to provide the corresponding acid chloride. The acid chloride is treated with a compound of Formula (I) in an appropriate solvent, optionally with heating, to provide compounds of Formula (II).

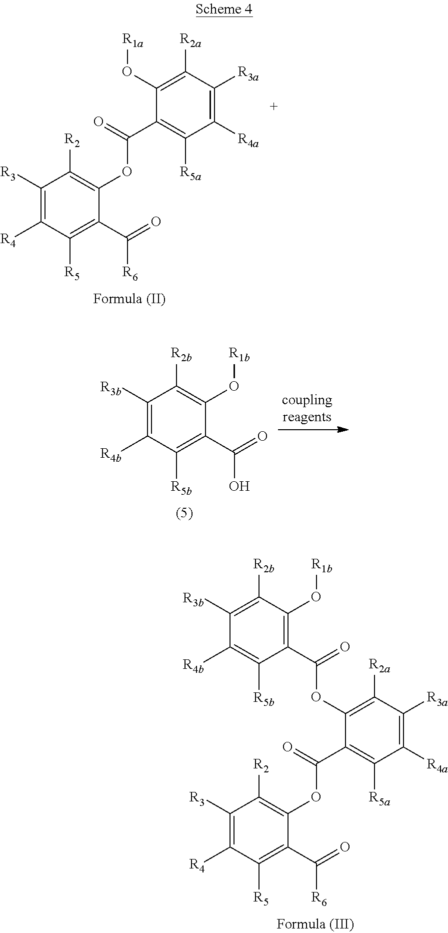

Compounds of Formula (III), wherein R₂, R₃, R₄, R₅, R₆, R₂ₐ, R₃ₐ, R₄ₐ, R₅ₐ, R_{1b}, R_{2b}, R_{3b}, R_{4b}, and R_{5b} are as defined in Formula (I) of the Summary section herein, are prepared as described in Scheme 4. Compounds of Formula (II) are treated with a benzoic acid of formula (5) in the presence of one or more coupling reagents, as disclosed in Scheme 1, in an appropriate solvent to provide compounds of Formula (III). Alternatively, a compound of formula (5) can be treated with a chlorinating agent (see Scheme 2) and a base including, but not limited to triethylamine or diisopropylethylamine, to provide the corresponding acid chloride. The acid chloride is treated with a compound of Formula (II) in an appropriate solvent, optionally with heating, to provide compounds of Formula (III).

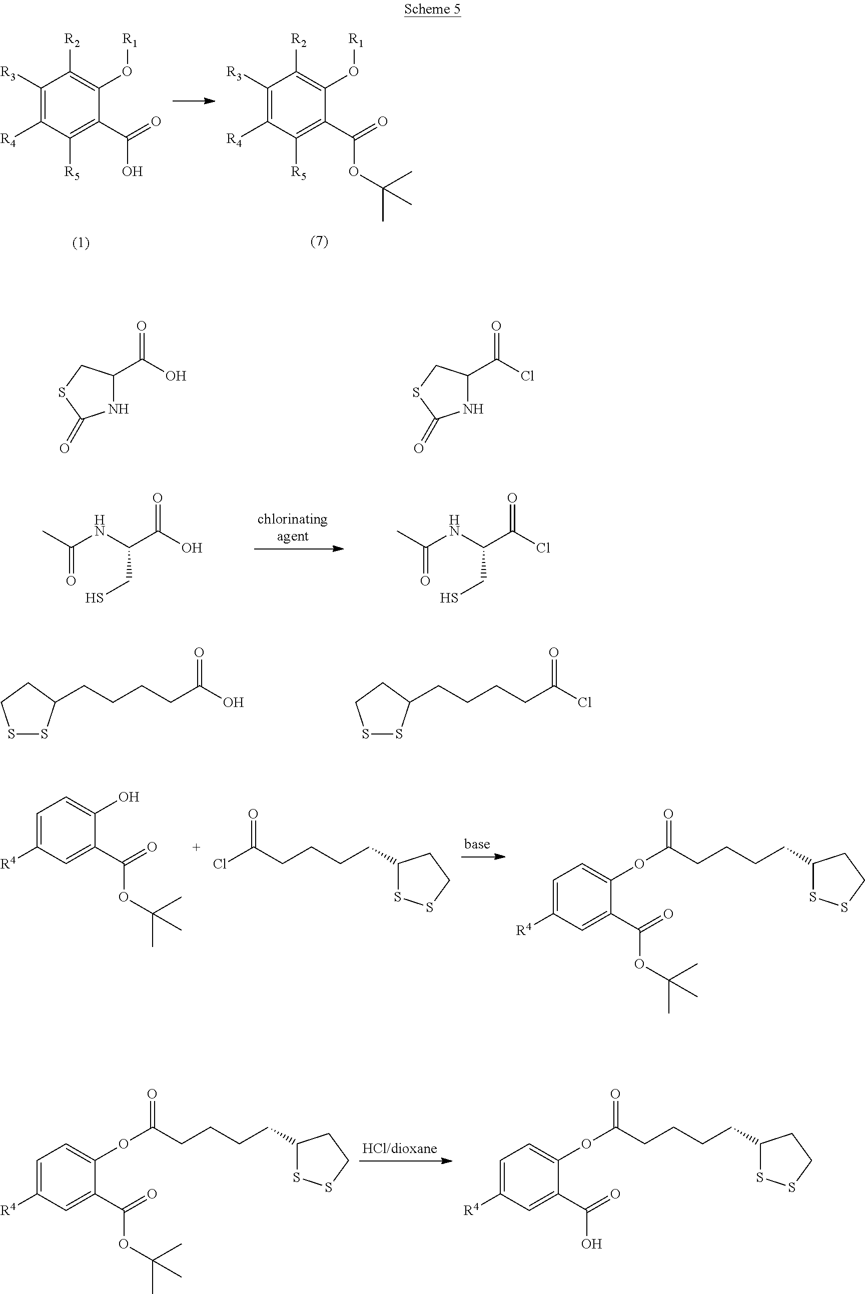

Alternatively, conjugates of Formula (I) can be prepared as described in Scheme 5. Compounds of formula (1), wherein R₁, R₂, R₃, R₄, and R₅ are as defined in Formula (I) in the Summary section, can be treated as described in Bull. Soc. Chim. France, pg 2985 (1974); and Applied Catalysis, 302 (1) pgs 42-47 (2006) to provide tert-butyl esters of formula (7). Antioxidants with carboxylic acid groups can be treated with a chlorinating agent (such as thionyl chloride or phosphorousoxy chloride (POCl₃) to provide the corresponding acid chlorides. Esters of formula (7) can be coupled to antioxidant acid chlorides in the presence of base (such as triethylamine or diisopropylethylamine) to provide conjugates of Formula (I).

For example, salicylic acid (R₄ is H) or diflunisal (R₄ is 2,4-difluorophenyl) can be converted into the corresponding tertbutyl ester using methods known in the art and then treated with the acid chloride of lipoic acid or NAC in the presence of base (triethylamine or diisopropylethylamine) to provide the salicylic acid-lipoic acid conjugate, diflunisal-lipoic acid conjugate, salnacedin, or diflunisal-NAC conjugate.

### Example 1

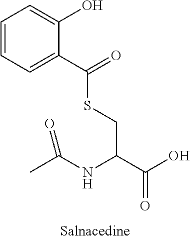 [SMILES: CC(=O)NC(CSC(=O)c1ccccc1O)C(=O)O ; "Salnacedine"]

### (R)-2-acetamido-3-(2-hydroxybenzoylthio)propanoic acid

The title compound is prepared using the procedures described in EP 0 080 229.

### Example 2

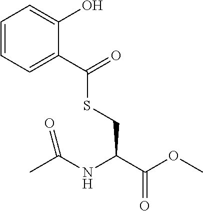 [SMILES: COC(=O)[C@H](CSC(=O)c1ccccc1O)NC(C)=O]

### (R)-methyl 2-acetamido-3-(2-hydroxybenzoylthio)propanoate

The title compound is prepared using similar procedures as described in EP 0 080 229.

### Example 3

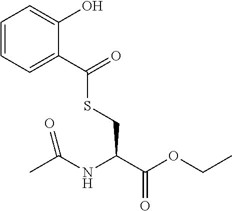 [SMILES: CCOC(=O)[C@H](CSC(=O)c1ccccc1O)NC(C)=O]

### (R)-ethyl 2-acetamido-3-(2-hydroxybenzoylthio)propanoate

The title compound is prepared using similar procedures as described in EP 0 080 229.

### Example 4

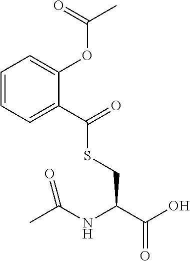 [SMILES: CC(=O)N[C@@H](CSC(=O)c1ccccc1OC(C)=O)C(=O)O]

### (R)-2-acetamido-3-(2-acetoxybenzoylthio)propanoic acid

The title compound is prepared using similar procedures as described in EP 0 080 229.

### Example 5

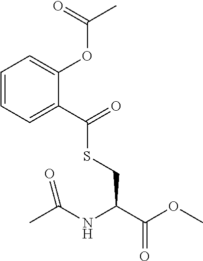 [SMILES: COC(=O)[C@H](CSC(=O)c1ccccc1OC(C)=O)NC(C)=O]

### (R)-methyl 2-acetamido-3-(2-acetoxybenzoylthio)propanoate

The title compound is prepared using similar procedures as described in EP 0 080 229.

### Example 6

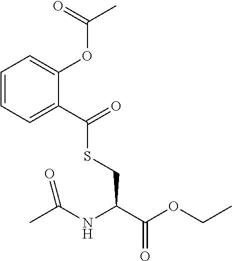 [SMILES: CCOC(=O)[C@H](CSC(=O)c1ccccc1OC(C)=O)NC(C)=O]

### (R)-ethyl 2-acetamido-3-(2-acetoxybenzoylthio)propanoate

The title compound is prepared using similar procedures as described in EP 0 080 229.

### Example 7

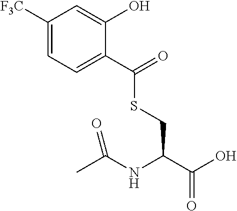 [SMILES: CC(=O)N[C@@H](CSC(=O)c1ccc(C(F)(F)F)cc1O)C(=O)O]

### (R)-2-acetamido-3-(2-hydroxy-4-(trifluoromethyl)benzoylthio)propanoic acid

The title compound is prepared using similar procedures as described in EP 0 080 229.

### Example 8

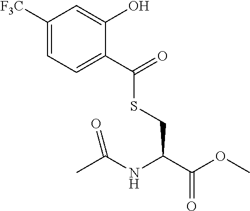 [SMILES: COC(=O)[C@H](CSC(=O)c1ccc(C(F)(F)F)cc1O)NC(C)=O]

### (R)-methyl 2-acetamido-3-(2-hydroxy-4-(trifluoromethyl)benzoylthio)propanoate

The title compound is prepared using similar procedures as described in EP 0 080 229.

### Example 9

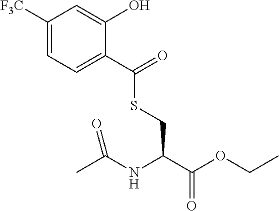 [SMILES: CCOC(=O)[C@H](CSC(=O)c1ccc(C(F)(F)F)cc1O)NC(C)=O]

### (R)-ethyl 2-acetamido-3-(2-hydroxy-4-(trifluoromethyl)benzoylthio)propanoate

The title compound is prepared using similar procedures as described in EP 0 080 229.

### Example 10

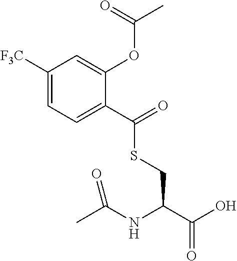 [SMILES: CC(=O)N[C@@H](CSC(=O)c1ccc(C(F)(F)F)cc1OC(C)=O)C(=O)O]

### (R)-2-acetamido-3-(2-acetoxy-4-(trifluoromethyl)benzoylthio)propanoic acid

The title compound is prepared using similar procedures as described in EP 0 080 229.

### Example 11

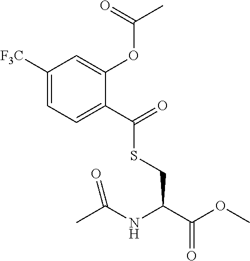 [SMILES: COC(=O)[C@H](CSC(=O)c1ccc(C(F)(F)F)cc1OC(C)=O)NC(C)=O]

### (R)-methyl 2-acetamido-3-(2-acetoxy-4-(trifluoromethyl)benzoylthio)propanoate

The title compound is prepared using similar procedures as described in EP 0 080 229.

### Example 12

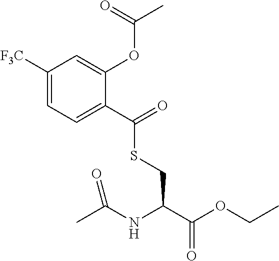 [SMILES: CCOC(=O)[C@H](CSC(=O)c1ccc(C(F)(F)F)cc1OC(C)=O)NC(C)=O]

### (R)-ethyl 2-acetamido-3-(2-acetoxy-4-(trifluoromethyl)benzoylthio)propanoate

The title compound is prepared using similar procedures as described in EP 0 080 229.

### Example 13

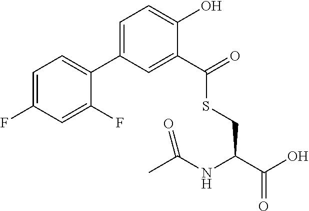 [SMILES: CC(=O)N[C@@H](CSC(=O)c1cc(-c2ccc(F)cc2F)ccc1O)C(=O)O]

### (R)-2-acetamido-3-(2′,4′-difluoro-4-hydroxybiphenylcarbonylthio)propanoic acid

The title compound is prepared using the procedures described in BE 900328.

### Example 14

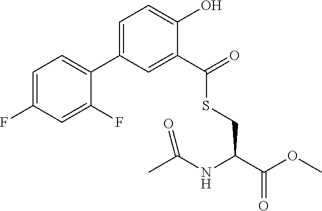 [SMILES: COC(=O)[C@H](CSC(=O)c1cc(-c2ccc(F)cc2F)ccc1O)NC(C)=O]

### (R)-methyl 2-acetamido-3-(2′,4′-difluoro-4-hydroxybiphenylcarbonylthio)propanoate

The title compound is prepared using similar procedures as described in BE 900328.

### Example 15

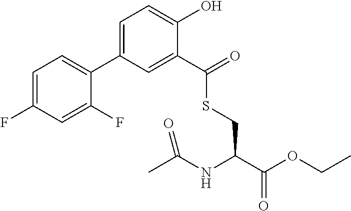 [SMILES: CCOC(=O)[C@H](CSC(=O)c1cc(-c2ccc(F)cc2F)ccc1O)NC(C)=O]

### (R)-ethyl 2-acetamido-3-(2′,4′-difluoro-4-hydroxybiphenylcarbonylthio)propanoate

The title compound is prepared using similar procedures as described in BE 900328.

### Example 16

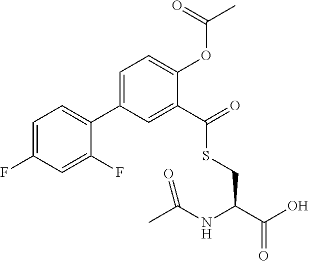 [SMILES: CC(=O)N[C@@H](CSC(=O)c1cc(-c2ccc(F)cc2F)ccc1OC(C)=O)C(=O)O]

### (R)-2-acetamido-3-(4-acetoxy-2′,4′-difluorobiphenylcarbonylthio)propanoic acid

The title compound is prepared using similar procedures as described in BE 900328.

### Example 17

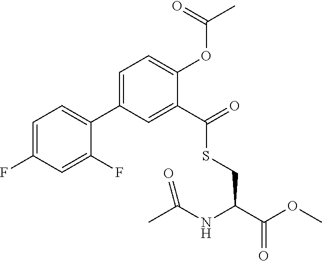 [SMILES: COC(=O)[C@H](CSC(=O)c1cc(-c2ccc(F)cc2F)ccc1OC(C)=O)NC(C)=O]

### (R)-methyl 2-acetamido-3-(4-acetoxy-2′,4′-difluorobiphenylcarbonylthio)propanoate

The title compound is prepared using similar procedures as described in BE 900328.

### Example 18

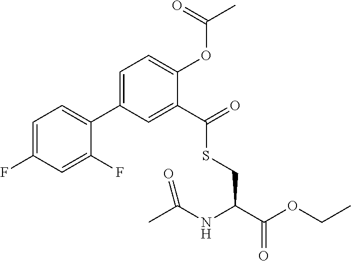 [SMILES: CCOC(=O)[C@H](CSC(=O)c1cc(-c2ccc(F)cc2F)ccc1OC(C)=O)NC(C)=O]

### (R)-ethyl 2-acetamido-3-(4-acetoxy-2′,4′-difluorobiphenylcarbonylthio)propanoate

The title compound is prepared using similar procedures as described in BE 900328.

### Example 19

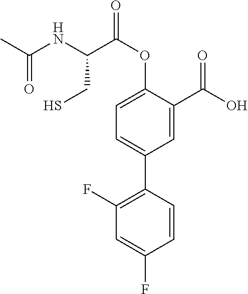 [SMILES: CC(=O)N[C@@H](CS)C(=O)Oc1ccc(-c2ccc(F)cc2F)cc1C(=O)O]

### (R)-4-(2-acetamido-3-mercaptopropanoyloxy)-2′,4′-difluorobiphenyl-3-carboxylic acid

The title compound is prepared using the procedures as described in Scheme 5.

### Example 20

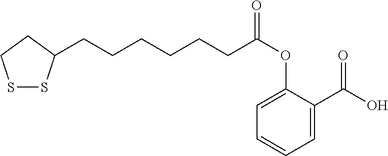 [SMILES: O=C(CCCCCCC1CCSS1)Oc1ccccc1C(=O)O]

### (R)-2-(5-(1,2-dithiolan-3-yl)pentanoyloxy)benzoic acid

The title compound is prepared using the procedures as described in Scheme 5.

### Example 21

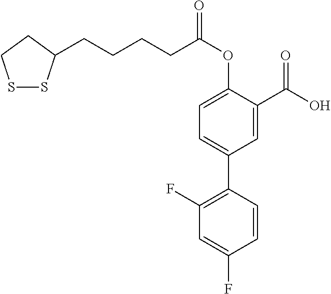 [SMILES: O=C(CCCCC1CCSS1)Oc1ccc(-c2ccc(F)cc2F)cc1C(=O)O]

### (R)-4-(5-(1,2-dithiolan-3-yl)pentanoyloxy)-2′,4′-difluorobiphenyl-3-carboxylic acid

The title compound is prepared using the procedures as described in Scheme 5.

### Biological Data

### Protection of β-Cell Failure and Prevention of Hyperglycemia in Streptozotocin Treated Rats

Diabetic mice or rats generated by streptozotocin administration exhibit an increase in levels of lipid peroxidation and a decrease in activity of antioxidant enzymes in the liver and kidneys as compared to control.

Conjugates of the present invention, such as salnacedin, administered orally and/or intraperitoneally (˜250 mg/kg) prior to a single dose of streptozotocin (45 mg/kg i.p.) in rats followed by 4 additional treatment days preserve β-cells, reducing the development of hyperglycemia. The blood glucose level in pretreated animals is lower than the control group associated with a preserve capacity of β-cell to secrete insulin measured in the blood.

Further, compounds of the present invention, such as Salnacedin, are tested for their efficiency at preserving β-cell function of mice challenged by one shot of streptozotocin (45 mg/kg i.p.). Oral or intraperitoneal administration of a conjugate of the present invention, such as Salnacedin, prior and during 5 days following streptozotocin exposition, protects β-cells from oxidative stress and reduces the development of hyperglycemia over time compared to control.

Compounds of the present invention, such as Salnacedin, reduce levels of 8-hydroxy-deoxyguanosine (8 OhdG) and malondialdehyde+4-hydroxy-2-nonenal (4HNE), markers for both oxidative stress and lipid peroxidation in the blood.

### Type 1 Diabetic Model in Mice

Mice induced by streptozotocin injection (120 mg/kg i.p.) are treated for 4 weeks with 250 mg/kg/day (oral or i.p.) of a compound of the present invention, such as salnacedin. At the end of the 4 week treatment, fasting glucose, fructosamine, triglycerides and cholesterol are measured. These biochemical parameters are reduced in comparison to control group. The reduction in these plasmatic parameters is more pronounced than observed with treatment of a salicylate or an antioxidant alone (e.g. salicyclic acid alone or N-acetylcysteine alone).

Further, oxidative stress and lipid peroxidation markers 8-hydroxy-deoxyguanosine (8 OhdG), malondialdehyde and 4-hydroxy-2-nonenal (4HNE) are also reduced.

Still further, inflammatory cytokines, such as TNFα and IL-6, and glutathione (GSH) levels in the liver and the kidney are reduced compared to non-treated animals.

Restoration of Insulin Sensibility in ob/ob and db/db Mice

Eight week old ob/ob and db/db mice are treated for 3 to 4 weeks with a daily dose of 250 mg/kg of a compound of the present invention, such as salnacedin, by oral gavage or with drug mix with food or subcutaneously. At the end of the 4 weeks, fasting blood glucose values are reduced compared to the control group or compared with ob/ob and db/db mice treated with a salicylate or an antioxidant alone (e.g. salicyclic acid alone or N-acetylcysteine alone).

Glucose tolerance test (OGTT or IPTT) provides a reduction in the elevation of glucose levels during the test compared to non treated animal. Additionally, the levels of insulin are measured 15 min following the glucose loading to determine the capacity of the β-cells to secrete insulin. The capacity of the β-cells to secrete insulin is greater in the group that's administered a compound of the present invention, such as salnacedin, compared to control demonstrating the protective effects toward pancreatic β-cells.

Further, compounds of the invention, including salnacedin, improve insulin sensitivity as evidenced by a sustained and pronounced glucose lowering effect. Also, the compounds of the invention, including salnacedin, provide a reduction in oxidative stress and lipid peroxidation as determined by the level of associated biomarkers: 8-hydroxy-deoxyguanosine (8 OhdG), malondialdehyde and 4-hydroxy-2-nonenal (4HNE). Finally, inflammatory cytokines, TNFα and IL-6, are reduced while the levels of glutathione (GSH) in the liver and the kidney are restored.

### Restoration of Insulin Sensitivity in Zucker Diabetic Fatty (ZDF) Rats

To assess whether conjugates comprising an antioxidant agent and an inflammatory agent would prevent glucose toxicity and progression of diabetes mellitus associated with β-cell failure overtime, we assessed whether compounds of Formula (I), including salnacedin, would alter the development of the disease in this Type 2 diabetic animal model.

Zucker diabetic rats from 6 to 12 weeks of age are treated daily with an oral dose of 250 mg/kg of a compound of the present invention, such as salnacedin.

Blood levels of 8 OhdG, malondialdehyde+4HNE, two markers of chronic oxidative stress and lipid peroxidation, are reduced in comparison to control animals.

Inflammatory cytokines, TNFα and IL6 are blunted when measured at the end of the 6 week treatment.

In comparison, placebo-treated or control animals develop progressive obesity, hyperglycemia, abnormal glucose tolerance test, defective glucose insulin secretion as well decrease islet insulin content.

Further, treatment with compounds of the present invention, including salnacedin, partially prevents the worsening of hyperglycemia, improves the glucose tolerance test, and preserves insulin secretion from β-cells. Fasting glucose, fructosamine, Hb1Ac, triglycerides and cholesterol are all reduced in comparison to the control group.

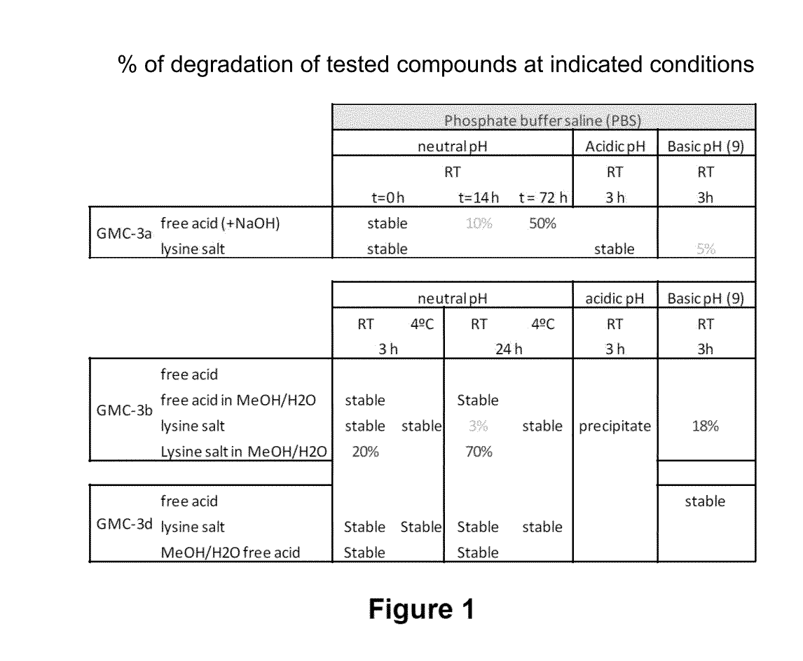

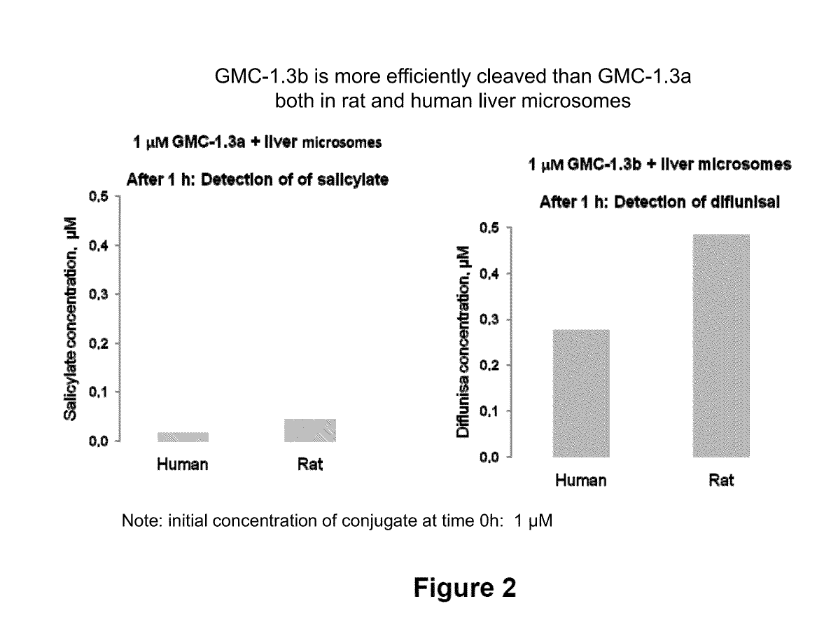

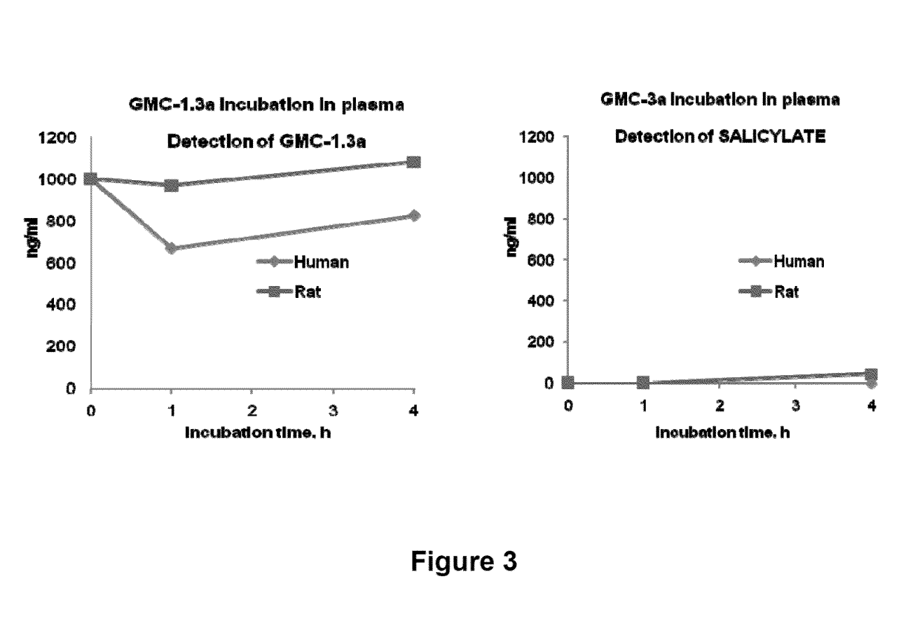

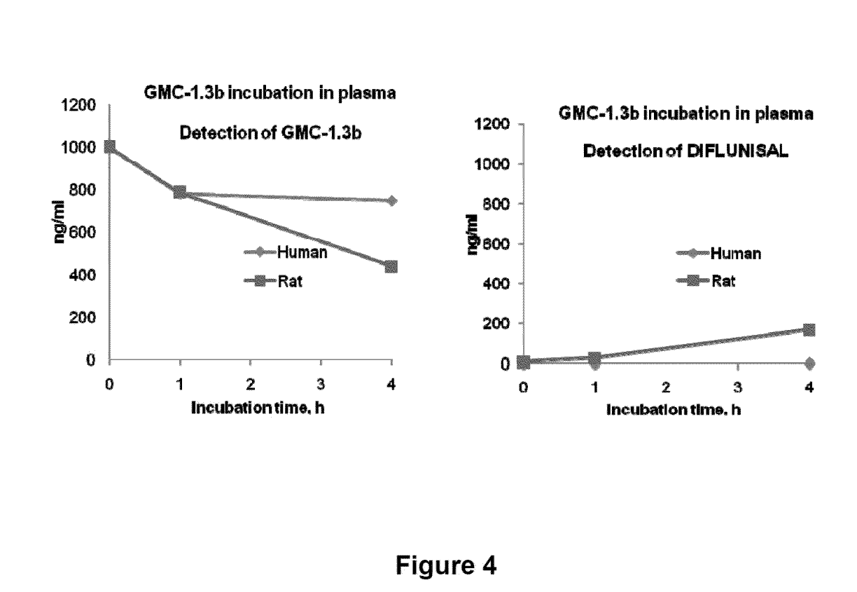

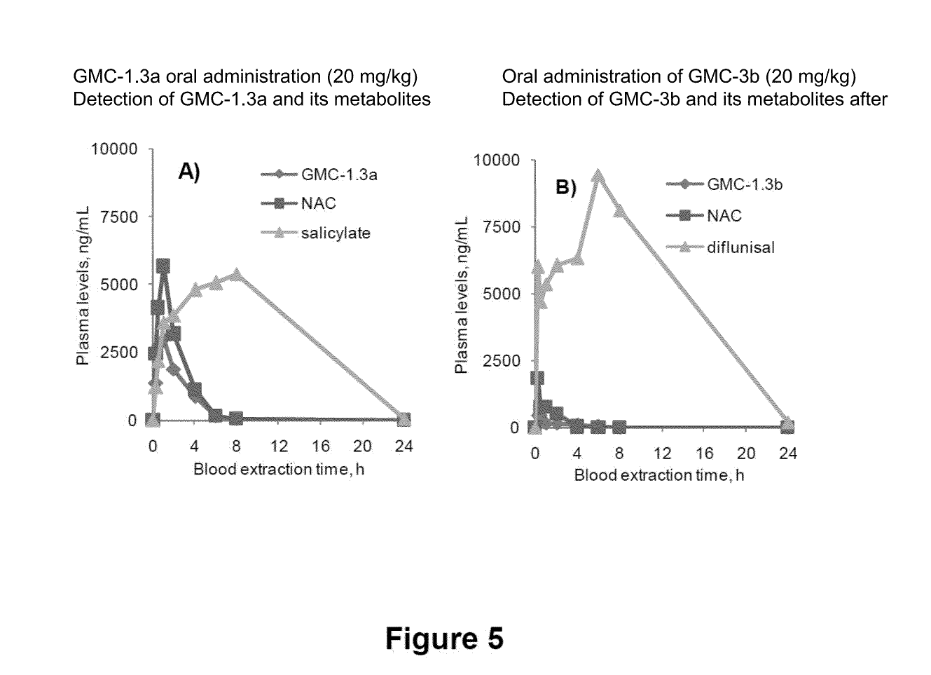

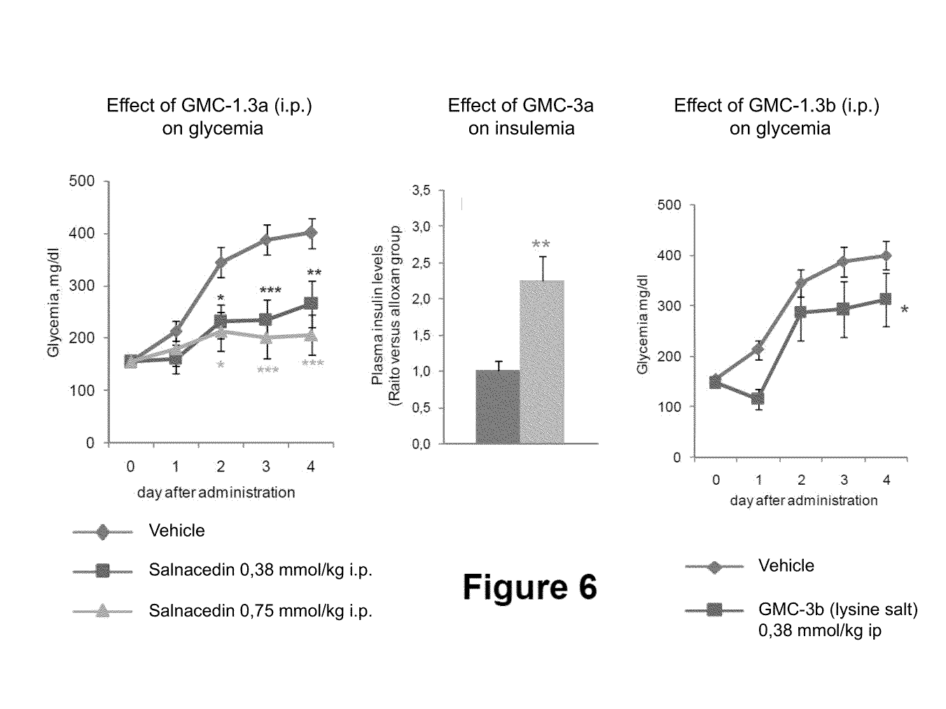

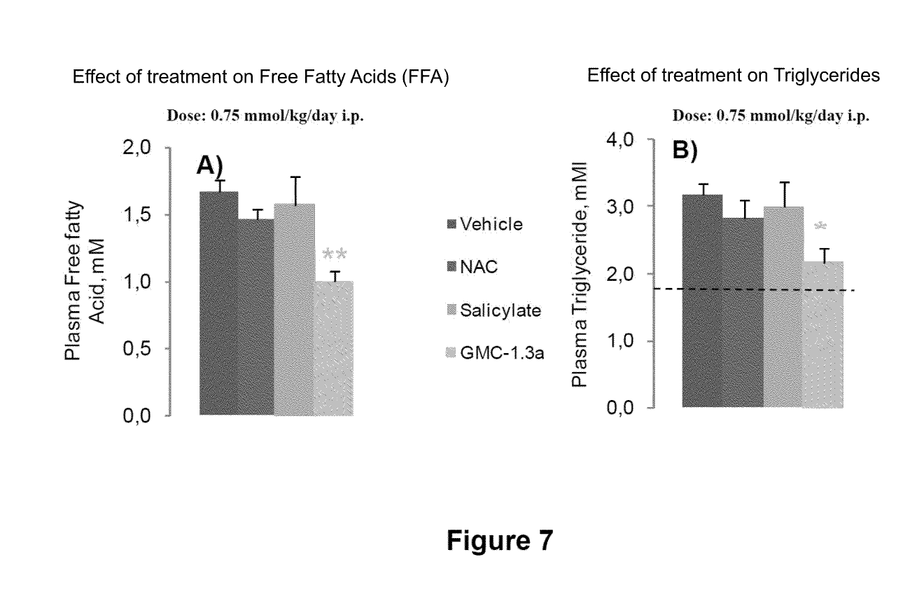

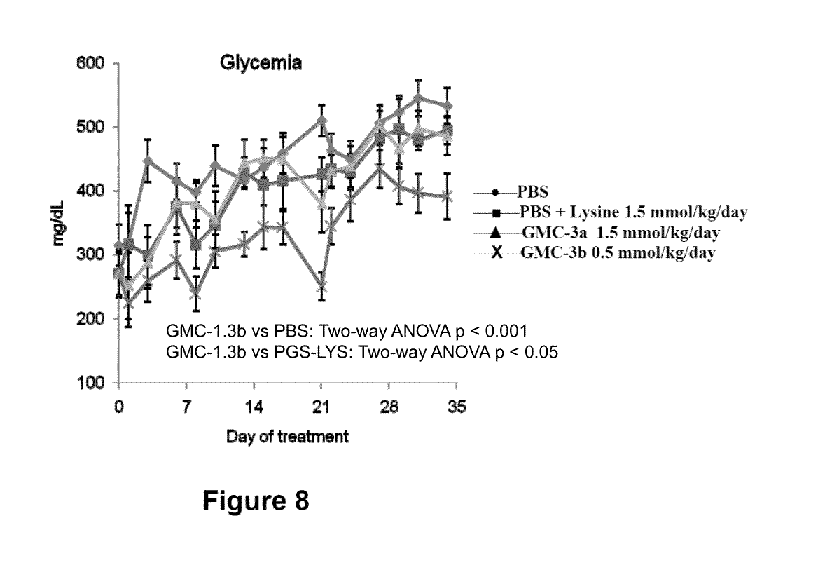

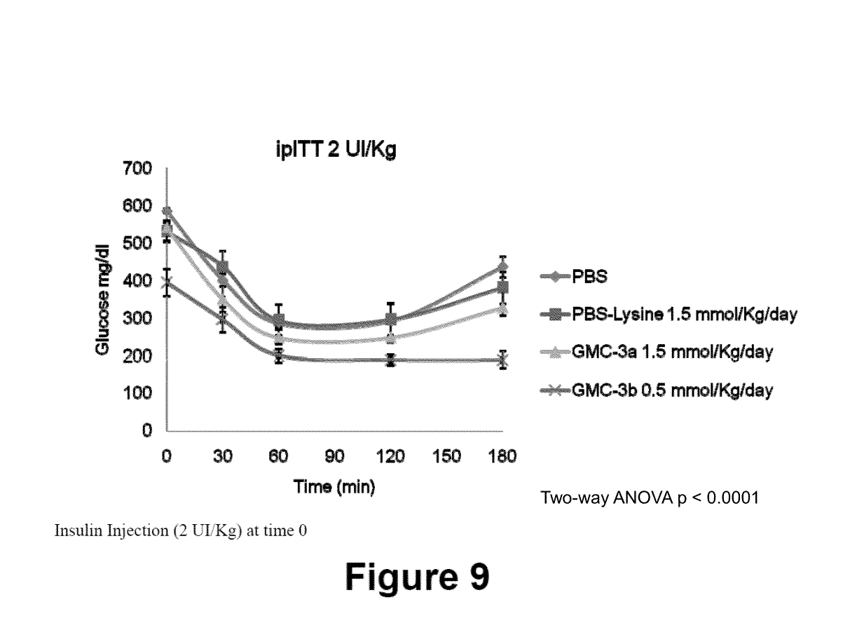

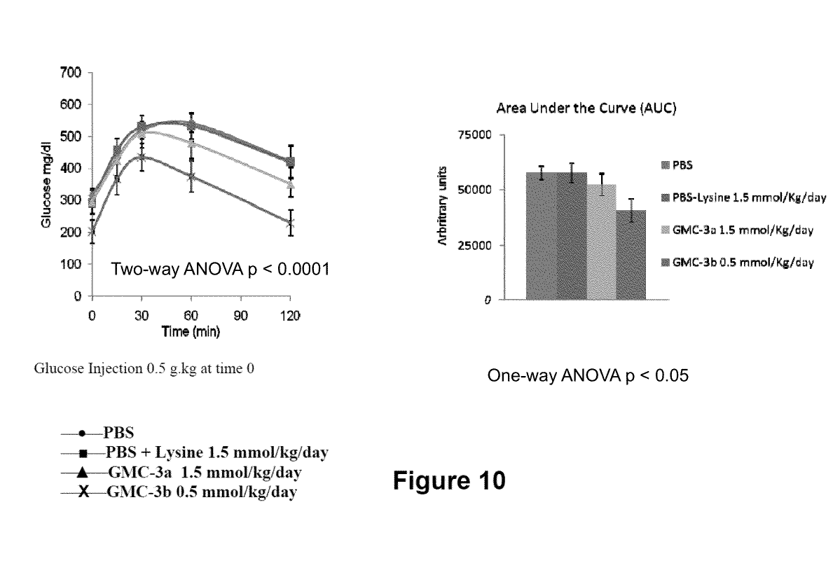

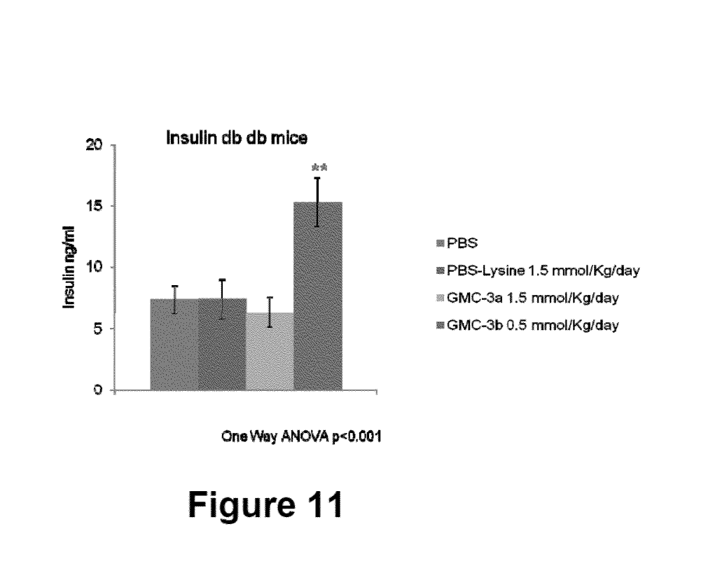

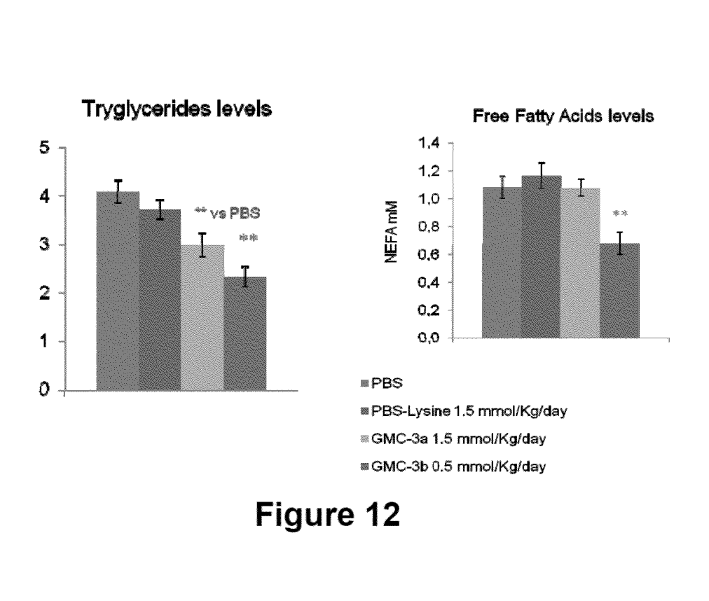

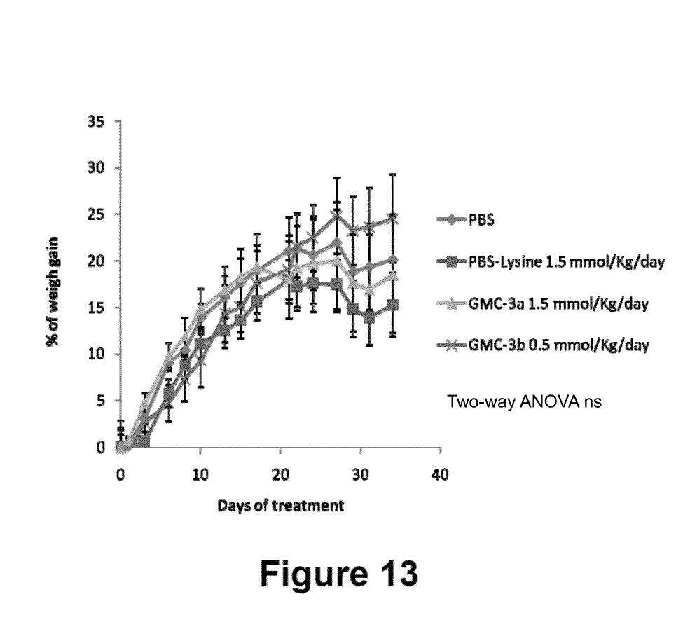

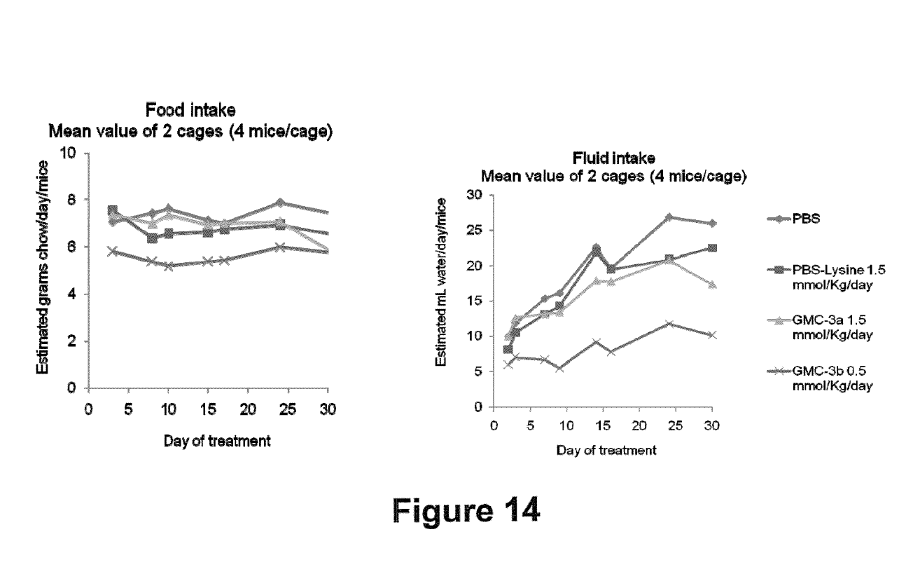

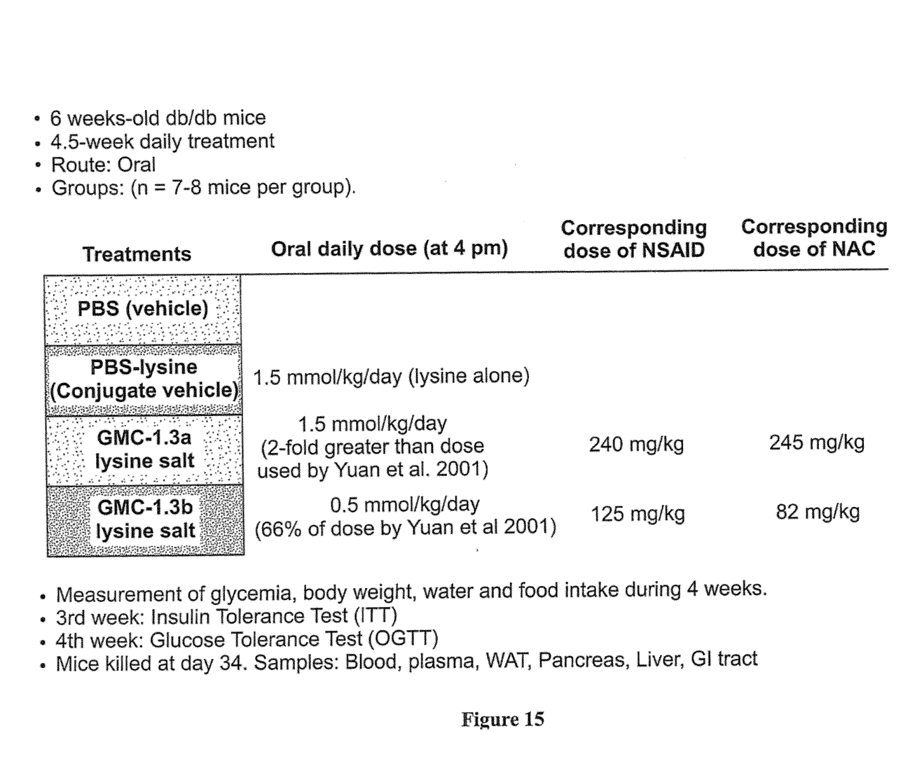

### Experimental Methods for FIGS. 1-15

Animals.

Male cd-1 mice weighing 25-30 g were purchased from Charles River Laboratories Spain. The animals were housed in animal quarters at 22° C. with a 12-h light/12-h dark cycle and fed ad libitum. 5-weeks old Male mice C57BL/Ks bearing the db/db mutation (The Jackson Laboratories) were purchased from Charles River Laboratories Spain (Sant Cugat del Valles, Spain).

Chemicals.

The chemicals N-Acetyl-cysteine and Sodium Salicylate were purchase from Sigma (Sigma Aldrich, St. Louis, Mo., USA) and PBS was purchase from Invitrogen. The compounds of diflunisal (GMC-1.3b), dexibuprofen (GMC-1.3d), and salnacedin (GMC-1.3a), and their lysine salts were purchase from Galchimia, S. L. (Galchimia S. L., A Coruna, Spain). All the compounds were dissolved in PBS, with lysine salt when indicated, and the pH of the compounds without lysine was adjusted with NaOH 6N until pH 7.

### In Vivo Beta Cell Protection Model

Beta-cell destruction was induced in cd-1 mice after 3 hours of fasting by a single intraperitoneal injection of a freshly prepared solution of alloxan 200 mg/kg (Sigma-Aldrich, San Luis, Mo.) that was dissolved in NaCl 0.9%. Single intraperitoneal drug administration was 1 hour before the alloxan administration. Animals received the different drugs dissolved in PBS pH 7.4, and the animals that not received any drug were injected with the vehicle, in this case PBS pH 7.4. At the end of the treatment, at day 4, animals were killed and the plasma collected and kept at −20° C. until used.

Chronic Treatment in db/db Mice.

The animals were treated with the indicated drugs for a month. The administration route was a single intraperitoneal injection. The glycemia levels were determined in blood from the Tail Vein, using a rapid glucose analyzer (Accu-Chek Aviva; Roche) 3 times per week, as body weight measure too. The food and water intake were measured twice a week. At the end of the treatment, the mice were sacrificed, in feeding state, with CO₂ euthanasia, and the blood was extracted from the Inferior Cave Vein, using heparin as an anticoagulant, and maintained at 4° C. until the preparation of plasma.

### Intraperitoneal Insulin Tolerance Test.

At the third week of treatment, an Insulin Tolerance Test was done to the mice in feeding state. The animals received an ip injection of Insulin 2 UI/kg (Humulin®). The glycemia levels were determined at the indicated time in blood from the Tail Vein, after the Insulin injection using a rapid glucose analyzer.

### Intraperitoneal Glucose Tolerance Test.

At the fourth week of treatment, a Glucose Tolerance Test was done to the mice after an overnight fasting. The animals received an ip injection of Glucose 0.5 g/kg (Glucosmon 50®). The glycemia levels were determined in blood at the indicated time from the Tail Vein after the Glucose injection using a rapid glucose analyzer.

Determination of Biochemical Parameters.

The circulating glucose concentration was determined by a rapid glucose analyzer (Accu-Chek Aviva; Roche). Plasma triglycerides and non esterified fatty acids were determined with standard colorimetric methods (Biosystems, Barcelona, Spain, and Wako Chemicals, Neuss, Germany, respectively). Plasma insulin concentration was determined by enzyme-linked immunosorbent assay method (CrystalChem, Downers Grove, Ill.).

Statistical Analysis.

Statistical comparisons between groups were established by two-way ANOVA using Prism 4 (GraphPad, San Diego, Calif.). A p value of less than 0.05 was considered to be statistically significant.

The data above shows the beneficial effects of compounds of the present invention, including salnacedin and diflunisal-NAC, in Type 2 diabetic animal models as compared to control or to animals treated with salicylate or an antioxidant alone (e.g. salicylic acid alone or N-acetylcysteine alone). The data described herein further provides that compounds of Formula (I), such as salnacedin and diflunisal-NAC, possess strong hypolipidemic and anti-diabetic effects as well as antioxidant properties in different animal models of diabetes useful in preventing the development of β-cell failure and aggravation of the diabetic status leading to cardiovascular complications. This data supports the therapeutic utility of conjugates comprising an antioxidant agent and an anti-inflammatory agent, such as salnacedin and diflunisal-NAC.

Moreover the additive and/or synergism effects of these conjugates allow for the decrease dosing of each independent active ingredient. These additive and/or synergistic effects reduce the liability of side effects associated with a salicylate agent, gastric bleeding, or an antioxidant, tinnitus, given to a patient alone.

We claim:

1. A method for treating atherosclerosis, neuropathy, nephropathy, retinopathy, inflammatory disorders, cardiovascular diseases, and metabolic disorders in a mammal or patient comprising administering to the mammal or patient in need of such treatment a compound of Formula (I) 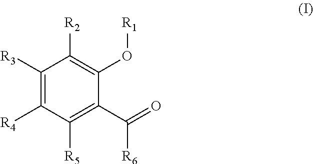
or a pharmaceutically acceptable salt thereof, wherein
R₁ is hydrogen, (C₁-C₆)alkylcarbonyl, or A;
R₂, R₃, R₄, and R₅ are independently hydrogen, (C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ₁Z₂, or (NZ₁Z₂)carbonyl, wherein the phenyl is optionally substituted with 1, 2, 3, 4, or 5 groups that are independently C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ₃Z₄, (NZ₃Z₄)carbonyl;
R₆ is —NZ₅Z₆, 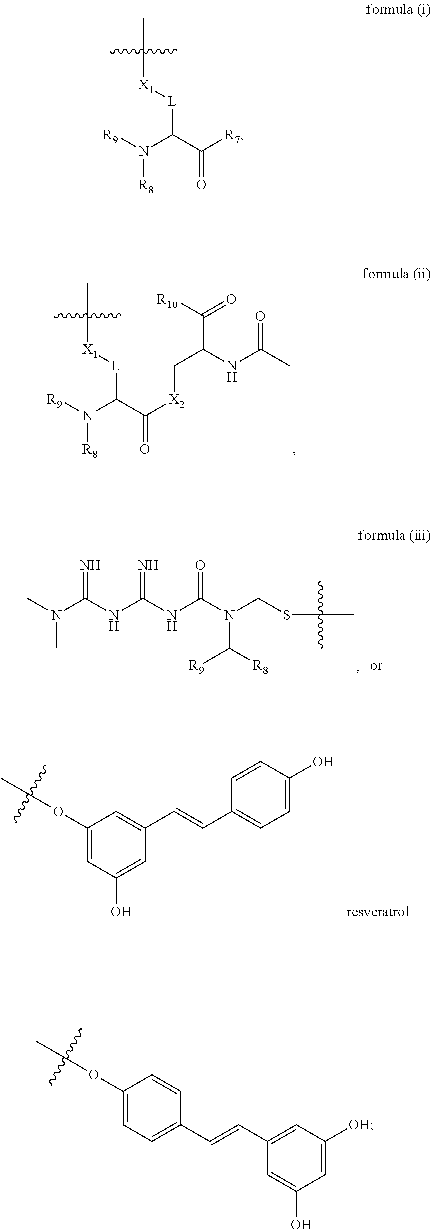
Z₅ and Z₆ are independently hydrogen, (C₁-C₆)alkyl, (C₁-C₆)alkylcarbonyl, phenyl, phenyl(CH₂)—, or phenyl(CH₂)₂—, wherein the phenyl is optionally substituted with 1, 2, 3, 4, or 5 groups that are independently (C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ₇Z₈, or (NZ₇Z₈)carbonyl;
Z₇ and Z₈ are independently hydrogen, (C₁-C₆)alkyl, or (C₁-C₆)alkylcarbonyl;
R₇ is (C₁-C₆)alkoxy, (C₁-C₆)alkyl, (C₁-C₆)alkylthio, hydroxy, or —NZ₉Z₁₀;
R₈ is hydrogen or (C₁-C₆)alkyl;
R₉ is hydrogen, (C₁-C₆)alkyl, or (C₁-C₆)alkylcarbonyl;
R₁₀ is (C₁-C₆)alkoxy, (C₁-C₆)alkyl, (C₁-C₆)alkylthio, hydroxy, or —NZ₉Z₁₀;
Z₉ and Z₁₀ are independently hydrogen, (C₁-C₆)alkyl, or (C₁-C₆)alkylcarbonyl;
X₁ and X₂ are independently O or S;
L is (C₁-C₆)alkylene;
A is 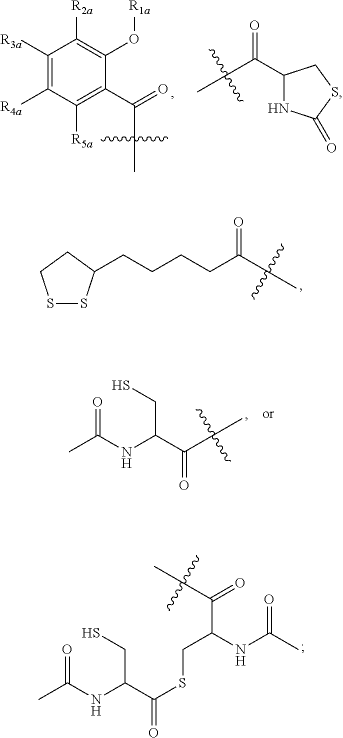
R₁ₐ is hydrogen, (C₁-C₆)alkylcarbonyl, or B;
R₂ₐ, R₃ₐ, R₄ₐ, and R₅ₐ are independently hydrogen, (C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ₁ₐZ₂ₐ, or (NZ₁ₐZ₂ₐ)carbonyl, wherein the phenyl is optionally substituted with 1, 2, 3, 4, or 5 groups that are independently C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ₃ₐZ₄ₐ, or (NZ₃ₐZ₄ₐ)carbonyl;
Z₁ₐ, Z₂ₐ, Z₃ₐ, and Z₄ₐ are independently hydrogen, (C₁-C₆)alkyl, or (C₁-C₆)alkylcarbonyl;
B is 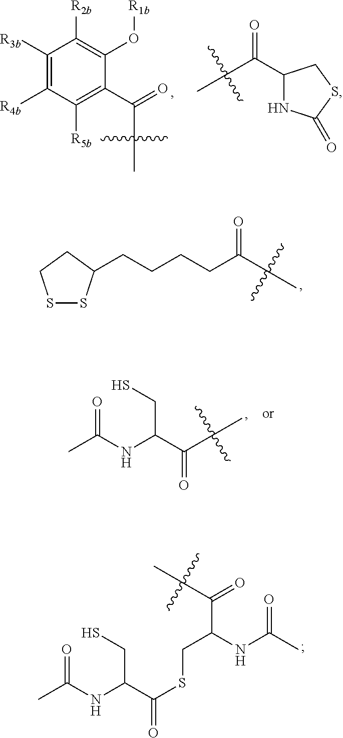
R_{1b} is hydrogen, (C₁-C₆)alkylcarbonyl, or C;
R_{2b}, R_{3b}, R_{4b}, and R_{5b} are independently hydrogen, (C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ_{1b}Z_{2b}, or (NZ_{1b}Z_{2b})carbonyl, wherein the phenyl is optionally substituted with 1, 2, 3, 4, or 5 groups that are independently C₁-C₆)alkoxy, (C₁-C₆)alkoxycarbonyl, (C₁-C₆)alkoxysulfonyl, (C₁-C₆)alkyl, (C₁-C₆)alkylcarbonyl, (C₁-C₆)alkylcarbonyloxy, (C₁-C₆)alkylsulfonyl, (C₁-C₆)alkylthio, carboxy, cyano, formyl, halo(C₁-C₆)alkoxy, halo(C₁-C₆)alkyl, halogen, hydroxy, hydroxy(C₁-C₆)alkyl, mercapto, nitro, phenyl, —NZ_{3b}Z_{4b}, or (NZ_{3b}Z_{4b})carbonyl;
Z_{1b}, Z_{2b}, Z_{3b}, and Z_{4b} are independently hydrogen, (C₁-C₆)alkyl, or (C₁-C₆)alkylcarbonyl; and
C is

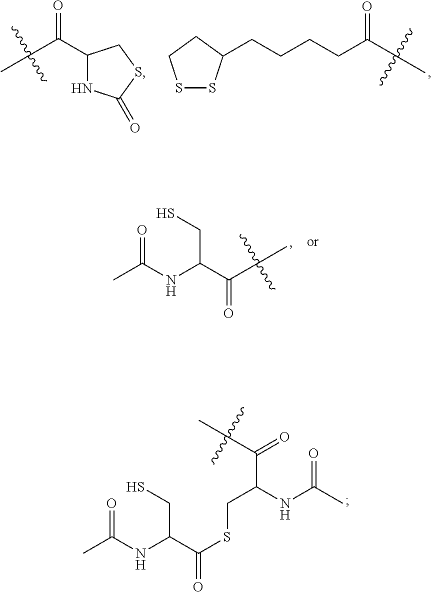

2. The method according to claim 1 wherein the metabolic disorders are dyslipidemia, insulin resistance, β-cell dysfunction, hyperglycemia, metabolic syndrome, and any form of diabetes mellitus including type I and type II diabetes.

3. The method according to claim 2 wherein
R₁ is hydrogen or acetyl;
R₂, R₃, R₄, and R₅ are independently hydrogen, trifluoromethyl, or 2,4-difluorophenyl.

4. The method according to claim 2 wherein
R₁ is hydrogen or acetyl;
R₂, R₃, R₄, and R₅ are independently hydrogen, trifluoromethyl, or 2,4-difluorophenyl;
R₇ is (C₁-C₆)alkoxy or hydroxy;
R₈ is hydrogen;
R₉ is (C₁-C₆)alkylcarbonyl;
X is S; and
L is CH₂.

5. The method according to claim 2 wherein
R₁ is hydrogen or acetyl;
R₂, R₃, R₄, and R₅ are independently hydrogen, trifluoromethyl, or 2,4-difluorophenyl;
R₇ is ethoxy, methoxy, or hydroxy;
R₈ is hydrogen;
R₉ is acetyl;
X is S; and
L is CH₂.

6. The method according to claim 2 wherein
R₁ is hydrogen or acetyl;
R₂, R₃, R₄, and R₅ are independently hydrogen, trifluoromethyl, or 2,4-difluorophenyl; and
R₆ is (L) N-acetylcysteine.

7. The method according to claim 2 wherein the compound of Formula (I) is Example 1, 2, 3, 4, 5, 6, 7, 8, 9, 10, 11, 12, 13, 14, 15, 16, 17, 18, 19, 20, 21.

8. The method according to claim 2 wherein the compound of Formula (I) is Example 1.

9. The method according to claim 2 wherein the compound of Formula (I) is Example 13.

10. The method according to claim 2 wherein the compound of Formula (I) is Example 19.

11. The method according to claim 2 wherein the compound of Formula (I) is Example 20.

12. The method according to claim 2 wherein the compound of Formula (I) is Example 21.

13. A method of reducing triglycerides and/or free fatty acids in a mammal or patient comprising administering to the mammal or patient in need of such treatment a therapeutically acceptable amount of a compound wherein the compound is Example 1, 2, 3, 4, 5, 6, 7, 8, 9, 10, 11, 12, 13, 14, 15, 16, 17, 18, 19, 20, or 21.

14. The method according to claim 13 wherein the compound is Example 1.

15. The method according to claim 13 wherein the compound is Example 13.

16. The method according to claim 13 wherein the compound is Example 19.

17. The method according to claim 13 wherein the compound is Example 20.

18. The method according to claim 13 wherein the compound is Example 21.
